SEQUENCE LISTING (32 sequences)

SEQ ID NO: 1 - protein, 16 aa, synthetic construct
  source 1..16: note = G12D(1-16)
MTEYKLVVVGADGVGK

SEQ ID NO: 2 - protein, 18 aa, synthetic construct
  source 1..18: note = G12D(2-19)
TEYKLVVVGADGVGKSAL

SEQ ID NO: 3 - protein, 21 aa, synthetic construct
  source 1..21: note = G12D(2-22)
TEYKLVVVGADGVGKSALTIQ

SEQ ID NO: 4 - protein, 28 aa, synthetic construct
  source 1..28: note = G12D(2-29)
TEYKLVVVGADGVGKSALTIQLIQNHFV

SEQ ID NO: 5 - protein, 16 aa, synthetic construct
  source 1..16: note = G12V(1-16)
MTEYKLVVVGAVGVGK

SEQ ID NO: 6 - protein, 18 aa, synthetic construct
  source 1..18: note = G12V(2-19)
TEYKLVVVGAVGVGKSAL

SEQ ID NO: 7 - protein, 15 aa, synthetic construct
  source 1..15: note = G12V(3-17)
EYKLVVVGAVGVGKS

SEQ ID NO: 8 - protein, 40 aa, synthetic construct
  source 1..40: note = G12V(3-42)
EYKLVVVGAVGVGKSALTIQLIQNHFVDEYDPTIEDSYRK

SEQ ID NO: 9 - protein, 10 aa, synthetic construct
  source 1..10: note = 7-16 G12D
VVVGADGVGK

SEQ ID NO: 10 - protein, 10 aa, synthetic construct
  source 1..10: note = 7-16 G12V
VVVGAVGVGK

SEQ ID NO: 11 - protein, 10 aa, synthetic construct
  source 1..10: note = 7-16 G12C
VVVGACGVGK

SEQ ID NO: 12 - protein, 9 aa, synthetic construct
  source 1..9: note = 8-16 G12D
VVGADGVGK

SEQ ID NO: 13 - protein, 9 aa, synthetic construct
  source 1..9: note = 8-16 G12V
VVGAVGVGK

SEQ ID NO: 14 - protein, 10 aa, synthetic construct
  source 1..10: note = 5-14 G12D
KLVVVGADGV

SEQ ID NO: 15 - protein, 10 aa, synthetic construct
  source 1..10: note = 10-19 G12D
GADGVGKSAL

SEQ ID NO: 16 - protein, 189 aa, synthetic construct [sequence omitted]

SEQ ID NO: 17 - protein, 188 aa, synthetic construct [sequence omitted]

SEQ ID NO: 18 - protein, 19 aa, synthetic construct
  source 1..19: note = P2A
ATNFSLLKQAGDVEENPGP

SEQ ID NO: 19 - protein, 18 aa, synthetic construct
  source 1..18: note = T2A
EGRGSLLTCGDVEENPGP

SEQ ID NO: 20 - protein, 22 aa, synthetic construct
  source 1..22: note = F2A
VKQTLNFDLLKLAGDVESNPGP

SEQ ID NO: 21 - protein, 20 aa, synthetic construct
  source 1..20: note = E2A
QCTNYALLKLAGDVESNPGP

SEQ ID NO: 22 - protein, 4 aa, synthetic construct
  source 1..4: note = Furin Cleavage Site
RAKR

SEQ ID NO: 23 - protein, 6 aa, synthetic construct
  REGION 1..2: note = The sequence comprises 1-100 alternating G or S subsequences wherein each subsequence can independently comprise 1-100 G.
GSGSGS

SEQ ID NO: 24 - protein, 6 aa, synthetic construct
  REGION 1..2: note = The sequence comprises 1-50 alternating G or S subsequences wherein each G or S subsequence can independently comprise 1-4 G and 0-1 S.
GSGSGS

SEQ ID NO: 25 - protein, 4 aa, synthetic construct
  REGION 1: note = The sequence can comprise 1-100 repeats of the amino acid G
GGGG

SEQ ID NO: 26 - protein, 6 aa, synthetic construct
  REGION 1..2: note = The sequence can comprise 1-100 repeats of the GA subsequence
GAGAGA

SEQ ID NO: 27 - protein, 6 aa, synthetic construct
  REGION 1..3: note = The sequence can comprise 1-100 repeats of the GGS subsequence
GGSGGS

SEQ ID NO: 28 - protein, 4 aa, synthetic construct
GGGG

SEQ ID NO: 29 - protein, 4 aa, synthetic construct
  REGION 1..4: note = The entire amino acid sequence can be repeated 1-100 times
GGGS

SEQ ID NO: 30 - protein, 8 aa, synthetic construct
  REGION 1..3: note = The amino acid sequence GGS can be repeated 1-100 times
  REGION 4..8: note = The amino acid sequence GGGGS can be repeated 1-100 times
GGSGGGGS

SEQ ID NO: 31 - protein, 15 aa, synthetic construct
GGGGSGGGGSGGGGS

SEQ ID NO: 32 - protein, 15 aa, synthetic construct
EAAAKEAAAKEAAAK

(19) United States
(12) Patent Application Publication
(10) Pub. No.: US 2025/0340868 A1
(43) Pub. Date: Nov. 6, 2025

(54) POLYNUCLEOTIDES ENCODING LINKED ANTIGENS AND USES THEREOF

(71) Applicant: STEMCELL TECHNOLOGIES CANADA INC., Vancouver (CA)

(72) Inventors: Scott McNabb Loughhead, Watertown, MA (US); Michael Finnan Maloney, Watertown, MA (US); Katarina Blagovic, Watertown, MA (US); Carolyne Kelly Smith, Watertown, MA (US)

(21) Appl. No.: 18/997,630

(22) PCT Filed: Jul. 28, 2023
(86) PCT No.: PCT/US2023/071263

§ 371 Date: Jan. 22, 2025

Related U.S. Application Data
(60) Provisional application No. 63/369,726, filed on Jul. 28, 2022.

(51) Int. Cl. C12N 15/11; C12N 5/078

(52) U.S. Cl. CPC C12N 15/11; C12N 5/0634; C12N 2710/20011; C12N 2730/10111; C12N 2740/16011

(57) ABSTRACT

The present disclosure provides isolated polynucleotides comprising multiple coding regions that are linked. In some aspects, a polynucleotide comprises at least a first coding region encoding a first antigen and a second coding region encoding a second antigen, wherein the first antigen and the second antigen are not the same, and wherein the first coding region and the second coding region are linked. The present disclosure also relates to the use of such polynucleotides to express multiple antigens in a cell and to induce multi-antigen-specific immune response in vivo.

### CROSS-REFERENCE TO RELATED APPLICATION

This PCT application claims the priority benefit of U.S. Provisional Application No. 63/369,726, filed Jul. 28, 2022, which is herein incorporated by reference in its entirety.

### REFERENCE TO SEQUENCE LISTING SUBMITTED ELECTRONICALLY

The content of the sequence listing is submitted electronically (Name: 4821_086PC01_SequenceListing_ST26.XML; Size: 36,597 bytes; and Date of Creation: Jul. 28, 2023) and is filed with the application herein incorporated by reference in its entirety.

### FIELD OF DISCLOSURE

The present disclosure relates generally to polynucleotides (e.g., isolated polynucleotides) comprising multiple nucleotide sequences encoding an antigen, wherein the multiple nucleotide sequences are linked (e.g., by a linker).

### BACKGROUND OF DISCLOSURE

Cancer remains one of the leading causes of death in the modern world. The standard treatments currently practiced in the clinic, including surgery, radiation, chemotherapy, and immunotherapy, have shown limited success. These therapies are usually only effective against early stage localized tumors and rarely against later staged, metastatic malignancies, leading to frequent relapse or eventual resistance to the therapy. Sharma, P., et al., Cell 168 (4): 707-723 (2017). Furthermore, various agents used in radiation and chemotherapy are damaging to normal tissues, which can lead to undesirable side effects. Accordingly, there remains a need for new treatment options with acceptable safety profile and high efficacy in cancer patients.

### BRIEF SUMMARY OF DISCLOSURE

Provided herein is an isolated polynucleotide comprising a single ORF with a first nucleotide sequence encoding a first antigen (“first coding region”) and a second nucleotide sequence encoding a second antigen (“second coding region”), wherein the first coding region and the second coding region are linked. In some aspects, the first coding region and the second coding region are linked by a linker.

In some aspects, the first coding region and the second coding region are arranged in the following order: first coding region is upstream of the second coding region. In some aspects, the order of the first coding region and the second coding region is different as compared to a corresponding order present in a reference polynucleotide, wherein the reference polynucleotide comprises a naturally-existing corresponding polynucleotide.

In some aspects, the single ORF of the polynucleotide further comprises one or more additional nucleotide sequences encoding an additional antigen (“additional coding region”). In some aspects, the additional antigen is: (i) not the same as the first antigen, (ii) not the same as the second antigen, or (iii) not the same as both the first antigen and the second antigen. In some aspects, the single ORF of the polynucleotide comprises at least two additional coding regions, at least three additional coding regions, at least four additional coding regions, at least five additional coding regions, at least six additional coding regions, at least seven additional coding regions, at least eight additional coding regions, at least nine additional coding regions, or at least ten additional coding regions. In some aspects, the additional coding region is linked to the first coding region or to the second coding region. In some aspects, the additional coding region is linked to the first coding region or to the second coding region by a linker.

In some aspects, the first coding region, the second coding region, and the additional coding region are arranged in the following order: (a) (first coding region)-L1-(second coding region)-L2-(additional coding region); (b) (first coding region)-L1-(additional coding region)-L2-(second coding region); or (c) (additional coding region)-L1-(first coding region)-L2-(second coding region); wherein L1 is a first linker and L2 is a second linker. In some aspects, the first linker and the second linker are the same. In some aspects, the first linker and the second linker are not the same. In some aspects, the order of the first coding region, the second coding region, and the third coding region is different as compared to a corresponding order present in the reference polynucleotide.

In some aspects, the first antigen is about 25 amino acids in length. In some aspects, the second antigen is about 25 amino acids in length. In some aspects, the additional antigen is about 25 amino acids in length. In some aspects, the first antigen comprises a cancer antigen, a non-self antigen, a self-antigen associated with a tumor, a disease associated-antigen, or combinations thereof. In some aspects, the second antigen comprises a cancer antigen, a non-self antigen, a self-antigen associated with a tumor, a disease associated-antigen, or combinations thereof. In some aspects, the additional antigen comprises a cancer antigen, a non-self antigen, a self-antigen associated with a tumor, a disease associated-antigen, or combinations thereof.

In some aspects, the cancer antigen comprises a KRAS antigen. In some aspects, the non-self antigen is derived from a pathogen selected from a human papillomavirus (HPV) antigen, a human immunodeficiency virus (HIV) antigen, a hepatitis B virus (HBV) antigen, or combinations thereof.

In some aspects, the KRAS antigen comprises an amino acid sequence which differs in sequence as compared to the amino acid sequence of a corresponding wild-type KRAS antigen. In some aspects, the amino acid sequence of the KRAS antigen has a sequence identity of less than about 99%, less than about 98%, less than about 97%, less than about 96%, less than about 95%, less than about 94%, less than about 93%, less than about 92%, less than about 91%, less than about 90%, less than about 85%, or less than about 90% as compared to the amino acid sequence of the corresponding wild-type KRAS antigen. In some aspects, the amino acid sequence of the KRAS antigen comprises an amino acid substitution selected from K5E, K5N, G10GG, G10V, G12A, G12C, G12D, G12F, G12I, G12L, G12R, G12S, G12V, G13C, G13D, G13E, G13R, G13V, V14I, L19F, T20M, Q22E, Q22H, Q22K, Q22R, Q25H, N26Y, F28L, E31K, D33E, P34L, P34Q, P34R, 136M, R41K, D57N, T58I, A59T, G60D, G60R, G60S, G60V, Q61A, Q61H, Q61K, Q61L, Q61P, Q61R, E63K, S65N, R68S, Y71H, T74A, L79I, R97I, Q99E, M111L, K117N, K117R, D119G, S122F, T144P, A146P, A146T, A146V, K147E, K147T, R149K, L159S, 1163S, R164Q, 1183N, 184M, or a combination thereof. In some aspects, the KRAS antigen comprises one or more of the following: G12D¹⁻¹⁶, a G12D²⁻¹⁹, a G12D²⁻²², a G12D²⁻²⁹, a G12V¹⁻¹⁶, a G12V²⁻¹⁹, a G12V³⁻¹⁷, or a G12V³⁻⁴² antigen.

In any of the above polynucleotides, in some aspects, the linker comprises a peptide linker. In some aspects, the peptide linker comprises a G4S linker or an EAAAK linker.

Some aspects of the present disclosure is related to an isolated polynucleotide comprising a single ORF with a first nucleotide sequence encoding a first antigen (“first coding region”), a second nucleotide sequence encoding a second antigen (“second coding region”), and a third nucleotide sequence encoding a third antigen (“third coding region”), wherein the first coding region is linked to the second coding region by a first linker, and wherein the second coding region is linked to the third coding region by a second linker. In some aspects, the first coding region, the second coding region, and the third coding region are arranged in an order, wherein the order is different as compared to a corresponding order present in a reference polynucleotide, wherein the reference polynucleotide comprises a naturally-existing corresponding polynucleotide.

Also provided herein is an isolated polynucleotide comprising a first nucleotide sequence encoding a first antigen (“first coding region”), a second nucleotide sequence encoding a second antigen (“second coding region”), a third nucleotide sequence encoding a third antigen (“third coding region”), and a fourth nucleotide sequence encoding a fourth antigen (“fourth coding region), wherein the first coding region is linked to the second coding region by a first linker, wherein the second coding region is linked to the third region by a second linker, and wherein the third coding region is linked to the fourth coding region by a third linker. In some aspects, the first coding region, the second coding region, the third coding region, and the fourth coding region are arranged in an order, wherein the order is different as compared to a corresponding order present in a reference polynucleotide, wherein the reference polynucleotide comprises a naturally-existing corresponding polynucleotide.

Also provided herein is an isolated polynucleotide comprising a first nucleotide sequence encoding a first antigen (“first coding region”), a second nucleotide sequence encoding a second antigen (“second coding region”), a third nucleotide sequence encoding a third antigen (“third coding region”), a fourth nucleotide sequence encoding a fourth antigen (“fourth coding region”), and a fifth nucleotide sequence encoding a fifth antigen (“fifth coding region”), wherein the first coding region is linked to the second coding region by a first linker, wherein the second coding region is linked to the third coding region by a second linker, wherein the third coding region is linked to the fourth coding region by a third linker, and wherein the fourth coding region is linked to the fifth coding region by a fourth linker. In some aspects, the first coding region, the second coding region, the third coding region, the fourth coding region, and the fifth coding region are arranged in an order, wherein the order is different as compared to a corresponding order present in a reference polynucleotide, wherein the reference polynucleotide comprises a naturally-existing corresponding polynucleotide.

Also provided herein is an isolated polynucleotide comprising a first nucleotide sequence encoding a first antigen (“first coding region”), a second nucleotide sequence encoding a second antigen (“second coding region”), a third nucleotide sequence encoding a third antigen (“third coding region”), a fourth nucleotide sequence encoding a fourth antigen (“fourth coding region”), a fifth nucleotide sequence encoding a fifth antigen (“fifth coding region”), and a sixth nucleotide sequence encoding a sixth antigen (“sixth coding region”), wherein the first coding region is linked to the second coding region by a first linker, wherein the second coding region is linked to the third coding region by a second linker, wherein the third coding region is linked to the fourth coding region by a third linker, wherein the fourth coding region is linked to the fifth coding region by a fourth linker, and wherein the fifth coding region is linked to the sixth coding region by a fifth linker. In some aspects, the first coding region, the second coding region, the third coding region, the fourth coding region, the fifth coding region, and the sixth coding region are arranged in an order, wherein the order is different as compared to a corresponding order present in a reference polynucleotide, wherein the reference polynucleotide comprises a naturally-existing corresponding polynucleotide. Also provided herein is an isolated polynucleotide comprising a first nucleotide sequence encoding a first antigen (“first coding region”), a second nucleotide sequence encoding a second antigen (“second coding region”), a third nucleotide sequence encoding a third antigen (“third coding region”), a fourth nucleotide sequence encoding a fourth antigen (“fourth coding region”), a fifth nucleotide sequence encoding a fifth antigen (“fifth coding region”), a sixth nucleotide sequence encoding a sixth antigen (“sixth coding region”), and a seventh nucleotide sequence encoding a seventh antigen (“seventh coding region”), wherein the first coding region is linked to the second coding region by a first linker, wherein the second coding region is linked to the third coding region by a second linker, wherein the third coding region is linked to the fourth coding region by a third linker, wherein the fourth coding region is linked to the fifth coding region by a fourth linker, wherein the fifth coding region is linked to the sixth coding region by a fifth linker, and wherein the sixth coding region is linked to the seventh coding region by a sixth linker. In some aspects, the first coding region, the second coding region, the third coding region, the fourth coding region, and the fifth coding region are arranged in an order, wherein the order is different as compared to a corresponding order present in a reference polynucleotide, wherein the Also provided herein is an isolated polynucleotide comprising a first nucleotide sequence encoding a first antigen (“first coding region”), a second nucleotide sequence encoding a second antigen (“second coding region”), a third nucleotide sequence encoding a third antigen (“third coding region”), a fourth nucleotide sequence encoding a fourth antigen (“fourth coding region”), a fifth nucleotide sequence encoding a fifth antigen (“fifth coding region”), and a sixth nucleotide sequence encoding a sixth antigen (“sixth coding region”), wherein the first coding region is linked to the second coding region by a first linker, wherein the second coding region is linked to the third coding region by a second linker, wherein the third coding region is linked to the fourth coding region by a third linker, wherein the fourth coding region is linked to the fifth coding region by a fourth linker, and wherein the fifth coding region is linked to the sixth coding region by a fifth linker. In some aspects, the first coding region, the second coding region, the third coding region, the fourth coding region, the fifth coding region, and the sixth coding region are arranged in an order, wherein the order is different as compared to a corresponding order present in a reference polynucleotide, wherein the reference polynucleotide comprises a naturally-existing corresponding polynucleotide. Also provided herein is an isolated polynucleotide comprising a first nucleotide sequence encoding a first antigen (“first coding region”), a second nucleotide sequence encoding a second antigen (“second coding region”), a third nucleotide sequence encoding a third antigen (“third coding region”), a fourth nucleotide sequence encoding a fourth antigen (“fourth coding region”), a fifth nucleotide sequence encoding a fifth antigen (“fifth coding region”), a sixth nucleotide sequence encoding a sixth antigen (“sixth coding region”), and a seventh nucleotide sequence encoding a seventh antigen (“seventh coding region”), wherein the first coding region is linked to the second coding region by a first linker, wherein the second coding region is linked to the third coding region by a second linker, wherein the third coding region is linked to the fourth coding region by a third linker, wherein the fourth coding region is linked to the fifth coding region by a fourth linker, wherein the fifth coding region is linked to the sixth coding region by a fifth linker, and wherein the sixth coding region is linked to the seventh coding region by a sixth linker. In some aspects, the first coding region, the second coding region, the third coding region, the fourth coding region, and the fifth coding region are arranged in an order, wherein the order is different as compared to a corresponding order present in a reference polynucleotide, wherein the Further provided herein is an isolated polynucleotide comprising a first nucleotide sequence encoding a first antigen (“first coding region”), a second nucleotide sequence encoding a second antigen (“second coding region”), a third nucleotide sequence encoding a third antigen (“third coding region”), a fourth nucleotide sequence encoding a fourth antigen (“fourth coding region”), a fifth nucleotide sequence encoding a fifth antigen (“fifth coding region”), a sixth nucleotide sequence encoding a sixth antigen (“sixth coding region”), a seventh nucleotide sequence encoding a seventh antigen (“seventh coding region”), and an eighth nucleotide sequence encoding an eighth antigen (“eighth coding region”), wherein the first coding region is linked to the second coding region by a first linker, wherein the second coding region is linked to the third coding region by a second linker, wherein the third coding region is linked to the fourth coding region by a third linker, wherein the fourth coding region is linked to the fifth coding region by a fourth linker, wherein the fifth coding region is linked to the sixth coding region by a fifth linker, wherein the sixth coding region is linked to the seventh coding region by a sixth linker, and wherein the seventh coding region is linked to the eighth coding region by a seventh linker. In some aspects, the first coding region, the second coding region, the third coding region, the fourth coding region, the fifth coding region, the sixth coding region, the seventh coding region, and the eighth coding region are arranged in an order, wherein the order is different as compared to a corresponding order present in a reference polynucleotide, wherein the reference polynucleotide comprises a naturally-existing corresponding polynucleotide.

Provided herein is an isolated polynucleotide comprising a first nucleotide sequence encoding a first antigen (“first coding region”), a second nucleotide sequence encoding a second antigen (“second coding region”), a third nucleotide sequence encoding a third antigen (“third coding region”), a fourth nucleotide sequence encoding a fourth antigen (“fourth coding region”), a fifth nucleotide sequence encoding a fifth antigen (“fifth coding region”), a sixth nucleotide sequence encoding a sixth antigen (“sixth coding region”), a seventh nucleotide sequence encoding a seventh antigen (“seventh coding region”), an eighth nucleotide sequence encoding an eighth antigen (“eighth coding region”), and a ninth nucleotide sequence encoding a ninth antigen (“ninth coding region”), wherein the first coding region is linked to the second coding region by a first linker, wherein the second coding region is linked to the third coding region by a second linker, wherein the third coding region is linked to the fourth coding region by a third linker, wherein the fourth coding region is linked to the fifth coding region by a fourth linker, wherein the fifth coding region is linked to the sixth coding region by a fifth linker, wherein the sixth coding region is linked to the seventh coding region by a sixth linker, wherein the seventh coding region is linked to the eighth coding region by a seventh linker, and wherein the eighth coding region is linked to the ninth coding region by a eighth linker. In some aspects, the first coding region, the second coding region, the third coding region, the fourth coding region, the fifth coding region, the sixth coding region, the seventh coding region, the eighth coding region, and the ninth coding region are arranged in an order, wherein the order is different as compared to a corresponding order present in a reference polynucleotide, wherein the reference polynucleotide comprises a naturally-existing corresponding polynucleotide.

Provided herein is an isolated polynucleotide comprising a first nucleotide sequence encoding a first antigen (“first coding region”), a second nucleotide sequence encoding a second antigen (“second coding region”), a third nucleotide sequence encoding a third antigen (“third coding region”), a fourth nucleotide sequence encoding a fourth antigen (“fourth coding region”), a fifth nucleotide sequence encoding a fifth antigen (“fifth coding region”), a sixth nucleotide sequence encoding a sixth antigen (“sixth coding region”), a seventh nucleotide sequence encoding a seventh antigen (“seventh coding region”), an eighth nucleotide sequence encoding an eighth antigen (“eighth coding region”), a ninth nucleotide sequence encoding a ninth antigen (“ninth coding region”), and a tenth nucleotide sequence encoding a tenth antigen (“tenth coding region”), wherein the first coding region is linked to the second coding region by a first linker, wherein the second coding region is linked to the third coding region by a second linker, wherein the third coding region is linked to the fourth coding region by a third linker, wherein the fourth coding region is linked to the fifth coding region by a fourth linker, wherein the fifth coding region is linked to the sixth coding region by a fifth linker, wherein the sixth coding region is linked to the seventh coding region by a sixth linker, wherein the seventh coding region is linked to the eighth coding region by a seventh linker, wherein the eighth coding region is linked to the ninth coding region by a eighth linker, and wherein the ninth coding region is linked to the tenth coding region by a ninth linker. In some aspects, the first coding region, the second coding region, the third coding region, the fourth coding region, the fifth coding region, the sixth coding region, the seventh coding region, the eighth coding region, the ninth coding region, and the tenth coding region are arranged in an order, wherein the order is different as compared to a corresponding order present in a reference polynucleotide, wherein the reference polynucleotide comprises a naturally-existing corresponding polynucleotide.

In any of the above polynucleotides, in some aspects, the first antigen is about 25 amino acids in length. In some aspects, the second antigen is about 25 amino acids in length. In some aspects, the third antigen is about 25 amino acids in length. In some aspects, the fourth antigen is about 25 amino acids in length. In some aspects, the fifth antigen is about 25 amino acids in length. In some aspects, the sixth antigen is about 25 amino acids in length. In some aspects, the seventh antigen is about 25 amino acids in length. In some aspects, the eighth antigen is about 25 amino acids in length. In some aspects, the ninth antigen is about 25 amino acids in length. In some aspects, the tenth antigen is about 25 amino acids in length.

In some aspects, the first antigen comprises a cancer antigen, a non-self antigen, a self-antigen associated with a tumor, a disease associated-antigen, or combinations thereof. In some aspects, the second antigen comprises a cancer antigen, a non-self antigen, a self-antigen associated with a tumor, a disease associated-antigen, or combinations thereof. In some aspects, the third antigen comprises a cancer antigen, a non-self antigen, a self-antigen associated with a tumor, a disease associated-antigen, or combinations thereof. In some aspects, the fourth antigen comprises a cancer antigen, a non-self antigen, a self-antigen associated with a tumor, a disease associated-antigen, or combinations thereof. In some aspects, the fifth antigen comprises a cancer antigen, a non-self antigen, a self-antigen associated with a tumor, a disease associated-antigen, or combinations thereof. In some aspects, the sixth antigen comprises a cancer antigen, a non-self antigen, a self-antigen associated with a tumor, a disease associated-antigen, or combinations thereof. In some aspects, the seventh antigen comprises a cancer antigen, a non-self antigen, a self-antigen associated with a tumor, a disease associated-antigen, or combinations thereof. In some aspects, the eighth antigen comprises a cancer antigen, a non-self antigen, a self-antigen associated with a tumor, a disease associated-antigen, or combinations thereof. In some aspects, the ninth antigen comprises a cancer antigen, a non-self antigen, a self-antigen associated with a tumor, a disease associated-antigen, or combinations thereof. In some aspects, the tenth antigen comprises a cancer antigen, a non-self antigen, a self-antigen associated with a tumor, a disease associated-antigen, or combinations thereof.

In some aspects, the cancer antigen comprises a KRAS antigen. In some aspects, the non-self antigen is derived from a pathogen selected from a human papillomavirus (HPV) antigen, a human immunodeficiency virus (HIV) antigen, a hepatitis B virus (HBV) antigen, or combinations thereof. In some aspects, the KRAS antigen comprises an amino acid sequence which differs in sequence as compared to the amino acid sequence of a corresponding wild-type KRAS antigen. In some aspects, the amino acid sequence of the KRAS antigen has a sequence identity of less than about 99%, less than about 98%, less than about 97%, less than about 96%, less than about 95%, less than about 94%, less than about 93%, less than about 92%, less than about 91%, less than about 90%, less than about 85%, or less than about 90% as compared to the amino acid sequence of the corresponding wild-type KRAS antigen. In some aspects, the amino acid sequence of the first KRAS antigen comprises an amino acid substitution selected from K5E, K5N, G10GG, G10V, G12A, G12C, G12D, G12F, G12I, G12L, G12R, G12S, G12V, G13C, G13D, G13F, G13R, G13V, V14I, L19F, T20M, Q22E, Q22H, Q22K, Q22R, Q25H, N26Y, F28L, E31K, D33E, P34L, P34Q, P34R, 136M, R41K, D57N, T58I, A59T, G60D, G60R, G60S, G60V, Q61A, Q61H, Q61K, Q61L, Q61P, Q61R, E63K, S65N, R68S, Y71H, T74A, L79I, R97I, Q99E, M111L, K117N, K117R, D119G, S122F, T144P, A146P, A146T, A146V, K147E, K147T, R149K, L159S, 1163S, R164Q, 1183N, 184M, or a combination thereof. In some aspects, the KRAS antigen comprises one or more of the following: G12D¹⁻¹⁶, a G12D²⁻¹⁹, a G12D²⁻²², a G12D²⁻²⁹, a G12V¹⁻¹⁶, a G12V²⁻¹⁹, a G12V³⁻¹⁷, or a G12V³⁻⁴² antigen.

In some aspects, any one of the first, second, third, fourth, fifth, sixth, seventh, eighth, or ninth linker comprises a peptide linker. In some aspects, the peptide linker comprises a GAS linker or an EAAAK linker.

For any of the polynucleotides described herein, in some aspects, the polynucleotide further comprises one or more of the following components: (1) an Internal Ribosome Entry Site (IRES), (2) an intron sequence, (3) a homology arm. (4) a promoter, (5) an enhancer, (6) a UTR, (7) a sequence encoding a signal peptide, (8) a translation initiation sequence, (9) a 3′ tailing region of linked nucleosides, (10) a 5′ cap, (11) a sequence encoding a 2A ribosome skip peptide, or (12) any combination of (1) to (11). In some aspects, the polynucleotide further comprises at least one modified nucleoside. In some aspects, the at least one modified nucleoside comprises 6-aza-cytidine, 2-thio-cytidine, α-thio-cytidine, pseudo-iso-cytidine, 5-aminoallyl-uridine, 5-iodo-uridine, N1-methyl-pseudouridine, 5,6-dihydrouridine, α-thio-uridine, 4-thio-uridine, 6-aza-uridine, 5-hydroxy-uridine, deoxy-thymidine, pseudo-uridine, inosine, α-thio-guanosine, 8-oxo-guanosine, O6-methyl-guanosine, 7-deaza-guanosine, N1-methyl adenosine, 2-amino-6-chloro-purine, N6-methyl-2-amino-purine, 6-chloro-purine, N6-methyl-adenosine, α-thio-adenosine, 8-azido-adenosine, 7-deaza-adenosine, pyrrolo-cytidine, 5-methyl-cytidine, N4-acetyl-cytidine, 5-methyl-uridine, 5-iodo-cytidine, or combinations thereof.

In some aspects, a polynucleotide described herein is a mRNA.

Also provided herein is a vector comprising any of the polynucleotides described herein. Some aspects of the present disclosure relates to cells comprising any of the polynucleotides described herein. In some aspects, the cell comprises a stem cell, somatic cell, or both. In some aspects, the stem cell comprises an induced pluripotent stem cell (iPSC), embryonic stem cell, tissue-specific stem cell, mesenchymal stem cell, or combinations thereof. In some aspects, the somatic cell comprises a blood cell. In some aspects, the blood cell comprises PBMC. In some aspects, the PBMC comprises an immune cell. In some aspects, the immune cell comprises a T cell, B cell, natural killer (NK) cell, dendritic cell (DC), NKT cell, mast cell, monocyte, macrophage, basophil, eosinophil, a neutrophil, DC2.4 dendritic cell, or combinations thereof. In some aspects, the cell has been passed through a constriction under a set of parameters, thereby causing a perturbation within the cell such that the polynucleotide entered the cell through the perturbation when contacted with the cell.

Also provided herein is a pharmaceutical composition comprising any of the polynucleotides, vectors, or cells described herein, and a pharmaceutically acceptable carrier. Also provided herein is a kit comprising any of the polynucleotides, vectors, or cells described herein.

Provided herein is a method of making a polynucleotide comprising enzymatically or chemically synthesizing any of the polynucleotides described herein.

Also provided herein is a method of inducing the expression of multiple antigens in a cell comprising intracellularly delivering the polynucleotide described herein to the cell. In some aspects, the multiple antigens are concurrently expressed in the cell after the intracellularly delivering. In some aspects, intracellularly delivering the polynucleotide to the cell comprises passing a cell suspension comprising the cell through a constriction under a set of parameters, thereby causing a perturbation within the cell such that the polynucleotide enters the cell through the perturbation when contacted with the cell.

In some aspects, the method further comprises contacting the cell with the polynucleotide. In some aspects, contacting the cell with the polynucleotide comprises incubating the cell suspension with the polynucleotide, such that the cell and the polynucleotide are in contact. In some aspects, the contacting occurs prior to passing the cell suspension through the constriction. In some aspects, the contacting occurs during the passing of the cell suspension through the constriction. In some aspects, the contacting occurs after the cell suspension passes through the constriction.

In some aspects, the set of parameters used to pass the cells through the constriction is selected from a cell density; pressure; length, width, and/or depth of the constriction; diameter of the constriction; diameter of the cells; temperature; entrance angle of the constriction; exit angle of the constriction; length, width, and/or width of an approach region; surface property of the constriction (e.g., roughness, chemical modification, hydrophilic, hydrophobic); operating flow speed; payload concentration; viscosity, osmolarity, salt concentration, serum content, and/or pH of the cell suspension; time in the constriction; shear rate in the constriction; type of payload, or combinations thereof.

In some aspects, the cell density is at least about 6×10⁷ cells/mL, at least about 7×10⁷ cells/mL, at least about 8×10⁷ cells/mL, at least about 9×10⁷ cells/mL, at least about 1×10⁸ cells/mL, at least about 1.1×10⁸ cells/mL, at least about 1.2×10⁸ cells/mL, at least about 1.3×10⁸ cells/mL, at least about 1.4×10⁸ cells/mL, at least about 1.5×10⁸ cells/mL, at least about 2.0×10⁸ cells/mL, at least about 3.0×10⁸ cells/mL, at least about 4.0×10⁸ cells/mL, at least about 5.0×10⁸ cells/mL, at least about 6.0×10⁸ cells/mL, at least about 7.0×10⁸ cells/mL, at least about 8.0×10⁸ cells/mL, at least about 9.0×10⁸ cells/mL, or at least about 1.0×10⁹ cells/mL, or more. In some aspects, the pressure is at least about 30 psi, at least about 35 psi, at least about 40 psi, at least about 45 psi, at least about 50 psi, at least about 55 psi, at least about 60 psi, at least about 65 psi, at least about 70 psi, at least about 75 psi, at least about 80 psi, at least about 85 psi, at least about 90 psi, at least about 95 psi, at least about 100 psi, at least about 110 psi, at least about 120 psi, at least about 130 psi, at least about 140 psi, or at least about 150 psi.

In some aspects, the constriction is contained within a microfluidic chip. In some aspects, the diameter of the constriction is about 20%, about 30%, about 40%, about 50%, about 60%, about 70%, about 80%, about 90%, or about 99% of the diameter of the cell. In some aspects, the width of the constriction is between about 0 μm to about 10 μm. In some aspects, the width of the constriction is less than about 1 μm, less than about 2 μm, less than about 3 μm, less than about 4 μm, less than about 5 μm, less than about 6 μm, less than about 7 μm, less than about 8 μm, less than about 9 μm, or less than about 10 μm. In some aspects, the length of the constriction is between about 0 μm to about 100 μm. In some aspects, the length of the constriction is less than about 0.1 μm, less than about 0.2 μm, less than about 0.3 μm, less than about 0.4 μm, less than about 0.5 μm, less than about 0.6 μm, less than about 0.7 μm, less than about 0.8 μm, less than about 0.9 μm, less than about 1 μm, less than about 2.5 μm, less than about 5 μm, less than about 7.5 μm, less than about 10 μm, less than about 12.5 μm, less than about 15 μm, less than about 20 μm, less than about 30 μm, less than about 40 μm, less than about 50 μm, less than about 60 μm, less than about 70 μm, less than about 80 μm, less than about 90 μm, or less than about 100 μm. In some aspects, the depth of the constriction is between at least about 1 μm to at least about 120 μm. In some aspects, the depth of the constriction is at least about 2 μm, at least about 3 μm, at least about 4 μm, at least about 5 μm, at least about 10 μm, at least about 20 μm, at least about 30 μm, at least about 40 μm, at least about 50 μm, at least about 60 μm, at least about 70 μm, at least about 80 μm, at least about 90 μm, at least about 100 μm, at least about 110 μm, or at least about 120 μm.

In some aspects, the cell suspension comprising the cell is passed through a plurality of constrictions. In some aspects, the plurality of constrictions comprise at least about 2, at least about 3, at least about 4, at least about 5, at least about 6, at least about 7, at least about 8, at least about 9, at least about 10, at least about 20, at least about 30, at least about 40, at least about 50, at least about 75, at least about 100, at least about 150, at least about 200, at least about 250, at least about 300, at least about 350, at least about 400, at least about 450, at least about 500, at least about 550, at least about 600, at least about 650, at least about 700, at least about 750, at least about 800, at least about 850, at least about 900, at least about 950, at least about 1,000 or more separate constrictions. In some aspects, each constriction of the plurality of constrictions is the same. In some aspects, one or more of the constrictions of the plurality of constrictions are different. In some aspects, the one or more of the constrictions differ in their length, depth, width, or combinations thereof.

Present disclosure also provides a method of inducing a multi-specific immune response in a subject in need thereof, comprising administering to the subject any of the polynucleotides, vectors, cells, or pharmaceutical compositions described herein. In some aspects, the multi-specific immune response comprises a CD8+ T cell response.

Also provided herein is a method of inducing an enhanced immune response in a subject in need thereof, comprising administering to the subject any of the polynucleotides, vectors, cells, or pharmaceutical compositions described herein to the subject. In some aspects, the enhanced immune response comprises: (i) an increase in the magnitude of the induced immune response as compared to a reference immune response, (ii) an increase in the breadth of the induced immune response as compared to a reference immune response, (iii) an increase in the duration of the induced immune response as compared to a reference immune response, or (iv) any combination of (i) to (iii); wherein the reference immune comprises the immune response observed in a corresponding subject that did not receive an administration of the polynucleotide or the modified cell.

Provided herein is a method of treating a disease or condition in a subject in need thereof, comprising administering to the subject any of the polynucleotides, vectors, cells, or pharmaceutical compositions described herein. In some aspects, the disease or condition comprises a cancer. In some aspects, the cancer is associated with abnormal KRAS expression. In some aspects, the disease or condition is associated with a non-self antigen. In some aspects, the non-self antigen is derived from a virus. In some aspects, the virus comprises a HPV, HIV, or HBV.

### BRIEF DESCRIPTION OF FIGURES

FIG. 1A shows three linked antigen mRNA constructs, each of which encodes about 25 amino acid long fragments of each of 5 antigens (HPV16 E6, HPV16 E7, CMV pp65, KRAS_{G12V}, and KRAS_{G12D}) arranged in a different order.

FIG. 1B shows activation of E6 TCR Jurkat cells (left graph), E7 TCR Jurkat cells (middle graph), and pp65-specific Cellero responder T cells (right graph) after co-culture with PMBCs squeezed with each of the three different linked antigen mRNA constructs shown in FIG. 1A. Controls shown include responder cells treated with one of the following: (i) non-modified PBMCs (i.e., no squeezing and no polynucleotide) (“NC”), (ii) PBMCs squeezed without any mRNA (“empty”), and (iii) NC PBMCs spiked with the respective minimal epitope (2^{nd} bar from the right for each of the different cell lines). Non-treated responder cells were also used as control (last bar from the right).

FIG. 2A shows three linked antigen mRNA constructs, two of which encodes about 25 amino acid long fragments of each of 10 antigens (HPV16 E6, HPV16 E7, CMV pp65, KRAS_{G12V}, KRAS_{G12D}, Flu M1, NY-ESO-1, HSV gD, SARS-COV2 S, and MAGE-A10) arranged in different orders, and the third mRNA construct encodes about 25 amino acid long fragments of each of 5 antigens (HPV16 E6, HPV16 E7, CMV pp65, KRAS_{G12V}, and KRAS_{G12D}).

FIG. 2B shows activation of E6 TCR Jurkat cells (left graph) and E7 TCR Jurkat cells (right graph) after co-culture with PMBCs squeezed with each of the three different linked antigen mRNA constructs shown in FIG. 2A. The controls are the same as those described in FIG. 1B.

FIG. 2C shows activation of pp65-specific Cellero responder T cells (left graph), M1-specific Flu Cellero responder T cells (middle graph), and NY-ESO-1-specific Cellero responder T cells (right graph) after co-culture with PMBCs squeezed with each of the three different linked antigen mRNA constructs shown in FIG. 2A. The controls are the same as those described in FIG. 1B.

FIG. 3A shows five linked antigen mRNA constructs, three of which encodes about 25 amino acid long fragments of each of 5 antigens (HPV16 E6, HPV16 E7, CMV pp65, KRAS_{G12V}, and KRAS_{G12D}) arranged in different orders, and two of which encodes about 25 amino acid long fragments of each of 10 antigens (HPV16 E6, HPV16 E7, CMV pp65, KRAS_{G12V}, KRAS_{G12D}, Flu M1, NY-ESO-1, HSV gD, SARS-COV2 S, and MAGE-A10) arranged in different orders.

FIG. 3B shows Western blotting of two SDS-PAGE gels on which were run samples of wheat germ extract translating each of the five linked antigen mRNA constructs shown in FIG. 3A. The gels were blotted with a primary antibody against HPV E6₂₉₋₃₈ SLP (clone 5G10) and a goat anti-rabbit secondary antibody.

FIG. 4 shows activation of KRAS_{G12V} TCR Jurkat cells after co-culture with PMBCs squeezed with each of the three different linked antigen mRNA constructs shown in FIG. 1A. The controls are the same as those described in FIG. 1B.

FIG. 5A shows schematic of the transduction of CD8 T cells with E6₂₉₋₃₈-specific TCR or E7₁₁₋₁₉-specific TCR expressing lentivirus, and activated by co-culture with PMBCs squeezed with either a linked antigen mRNA, a linked antigen mRNA and Signal 2/3 mRNAs, or Signal 2/3 mRNAs alone. Cells were cultured 6 days before restimulation with the corresponding E6₂₉₋₃₈ or E7₁₁₋₁₉ peptide and measuring cytokine production via intracellular cytokine staining (ICS).

FIG. 5B shows activation of E6 TCR-transduced and E7 TCR-transduced CD8+ T cells after co-culture with PMBCs squeezed with a linked antigen mRNA and Signal 2/3 mRNAs together compared to either the linked antigen mRNA or Signal 2/3 mRNAs alone.

FIGS. 6A and 6B show G12V and G12D specific responses, respectively, in A*11-restricted G12V and G12D TCR Jurkat responder cells cultured with PBMCs squeezed with one of the following mRNA constructs: (1) linked mRNA construct encoding: (a) ˜25 aa fragments (overlapping with the 1-25 aa sequence in the whole KRAS protein and mutations on the 12^{th} codon) KRAS_{G12D} and KRAS_{G12V} antigens, (b) CD86 (i.e., signal 2), (c) membrane-bound IL-2 (i.e., signal 3), and (d) membrane-bound IL-12 (i.e., signal 3) (“Linked Antigen+2/3”); (2) linked mRNA construct encoding only the ˜25 aa fragments (overlapping with the 1-25 aa sequence in the whole KRAS protein and mutations on the 12^{th} codon) KRAS_{G12D} and KRAS_{G12V} antigens (“G12D-G12V Linked Antigen”); (3) linked mRNA construct encoding only CD86 (i.e., signal 2), (c) membrane-bound IL-2 (i.e., signal 3), and (d) membrane-bound IL-12 (i.e., signal 3) (“signal 2/3”); (4) linked mRNA construct encoding: (a) single ˜25 aa fragment comprising the G12V mutation, (b) CD86 (i.e., signal 2), (c) membrane-bound IL-2 (i.e., signal 3), and (d) membrane-bound IL-12 (i.e., signal 3) (“G12V+2/3”); (5) linked mRNA construct encoding: (a) single ˜25 aa fragment comprising the G12D mutation, (b) CD86 (i.e., signal 2), (c) membrane-bound IL-2 (i.e., signal 3), and (d) membrane-bound IL-12 (i.e., signal 3) (“G12D-2/3”), (6) linked mRNA construct encoding: (a) ˜25 aa fragment overlapping with the first 1-25 aa of native wild-type KRAS; (b) CD86 (i.e., signal 2), (c) membrane-bound IL-2 (i.e., signal 3), and (d) membrane-bound IL-12 (i.e., signal 3) (“WT+2/3”); (7) non-linked mRNA construct encoding only single ˜25 aa fragment comprising the G12V mutation (i.e., no signals 2 and 3) (“G12V”); (8) non-linked mRNA construct encoding only single-25 aa fragment comprising the G12D mutation (i.e., no signals 2 and 3) (“G12D”); and (9) non-linked mRNA construct encoding only ˜25 aa fragment overlapping with the first 1-25 aa of native wild-type KRAS (i.e., no signals 2 and 3) (“Wild Type”). The G12V and G12D specific responses are shown as evidenced by luminescence expression. The specific values shown above some of the bars represent the fold-change over the luminescence expression observed in responder cells cultured with PBMCs squeezed with no mRNA (“empty”).

FIGS. 7A and 7B show G12V and G12D specific responses, respectively, in A*11-restricted G12V and G12D TCR Jurkat responder cells cultured with PBMCs squeezed a mRNA construct encoding seven linked KRAS mutant antigens. The mRNA constructs were used at one of two doses (250 μg/mL and 500 μg/mL) and included the following: (1) linked mRNA construct encoding only ˜25 aa fragments (overlapping with the 1-25 aa sequence in the whole KRAS protein and a mutation on the 12^{th} codon) KRAS_{G12D}, KRAS_{G12V}, KRAS_{G12C}, KRAS_{G13D}, KRAS_{G12A}, KRAS_{G12R} and KRAS_{G12S} antigens (“7 mut_v1”); and (2) linked mRNA construct encoding only ˜25 aa fragments (overlapping with the 1-25 aa sequence in the whole KRAS protein and a mutation on the 12^{th} codon) KRAS_{G12S}, KRAS_{G12R}, KRAS_{G12A}, KRAS_{G13D}, KRAS_{G12C}, KRAS_{G12V} and KRAS_{G12D} antigens (“7mut_v2”). The G12V and G12D specific responses are shown as evidenced by luminescence expression. The specific values shown above some of the bars represent the fold-change over the luminescence expression observed in responder cells cultured with PBMCs squeezed with no mRNA (“empty”).

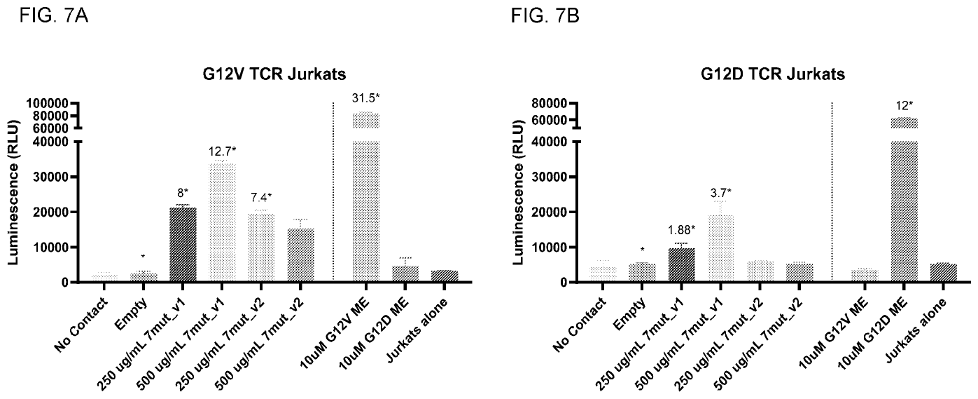

FIGS. 8A and 8B show G12V and G12D specific responses, respectively, in A*11-restricted G12V and G12D TCR Jurkat responder cells cultured with PBMCs squeezed a mRNA construct encoding seven linked KRAS mutant antigens and further encoding signals 2 (i.e., CD86) and 3 (i.e., membrane-bound IL-2 and IL-12). The specific mRNA constructs were as follows: (1) linked mRNA construct encoding: (a) ˜25 aa fragments (overlapping with the 1-25 aa sequence in the whole KRAS protein and a mutation on the 12^{th} codon) KRAS_{G12D}, KRAS_{G12V}, KRAS_{G12C}, KRAS_{G13D}, KRAS_{G12A}, KRAS_{G12R} and KRAS_{G12S} antigens (termed “KRAS 7mut_v1”), (b) CD86 (i.e., signal 2), (c) membrane-bound IL-2 (i.e., signal 3), and (d) membrane-bound IL-12 (i.e., signal 3) (“7mut_v1+Signal 2/3”); (2) linked mRNA construct encoding only the ˜25 aa fragments (overlapping with the 1-25 aa sequence in the whole KRAS protein and a mutation on the 12^{th} codon) KRAS_{G12D}, KRAS_{G12V}, KRAS_{G12C}, KRAS_{G13D}, KRAS_{G12A}, KRAS_{G12R} and KRAS_{G12S} antigens (“7mut_v1”); (3) linked mRNA construct encoding: (a) ˜25 aa fragments (overlapping with the 1-25 aa sequence in the whole KRAS protein and a mutation on the 12^{th} codon) KRAS_{G12S}, KRAS_{G12R}, KRAS_{G12A}, KRAS_{G13D}, KRAS_{G12C}, KRAS_{G12V} and KRAS_{G12D} antigens (termed KRAS 7mut_v2 here), (b) CD86 (i.e., signal 2), (c) membrane-bound IL-2 (i.e., signal 3), and (d) membrane-bound IL-12 (i.e., signal 3) (“7mut_v2+Signal 2/3”): (4) linked mRNA construct encoding only the ˜25 aa fragments (overlapping with the 1-25 aa sequence in the whole KRAS protein and a mutation on the 12^{th} codon) KRAS_{G12S}, KRAS_{G12R}, KRAS_{G12A}, KRAS_{G13D}, KRAS_{G12C}, KRAS_{G12V} and KRAS_{G12D} antigens (“mut_v2”); and (5) linked mRNA construct encoding only CD86 (i.e., signal 2), (c) membrane-bound IL-2 (i.e., signal 3), and (d) membrane-bound IL-12 (i.e., signal 3) (“signal 2/3 alone”). The G12V and G12D specific responses are shown as evidenced by luminescence expression. The specific values shown above some of the bars represent the fold-change over the luminescence expression observed in responder cells cultured with PBMCs squeezed with no mRNA (“empty”).

### DETAILED DESCRIPTION OF DISCLOSURE

The present disclosure is generally directed to isolated polynucleotides that can be used to induce the expression of multiple antigens in a cell. More particularly, provided herein are polynucleotides comprising at least a first coding region encoding a first antigen and a second coding region encoding a second antigen, wherein the first antigen and the second antigen are not the same, and wherein the first coding region and the second coding region are linked (e.g., by a linker). As described and demonstrated herein, such polynucleotides can be intracellularly delivered to the cells, wherein the cell subsequently express both the first antigen and the second antigen.

As further described herein, the exemplary delivery methods provided herein (i.e., squeeze delivery) have certain distinct properties that are not shared by other non-constriction mediated delivery methods known in the art. For example, in addition to the improved ability to deliver various types of payloads into a cell, the squeeze processing methods described herein exert minimal lasting effects on the cells Compared to traditional delivery methods such as electroporation, the squeeze processing methods of the present disclosure preserve both the structural and functional integrity of the squeezed cells. Contrary to the delivery methods provided herein, electroporation can induce broad and lasting alterations in gene expression, which can lead to non-specific activation of cells (e.g., human T cells) and delayed proliferation upon antigen stimulation. With the present methods, any alterations to the cells (e.g., perturbations in the cell membrane) is transient and the perturbations are rescaled once the cells are removed from the constriction. Non-limiting examples of the various aspects are shown in the present disclosure.

### I. General Techniques

Some of the techniques and procedures described or referenced herein are generally well understood and commonly employed using conventional methodology by those skilled in the art, such as, for example, the widely utilized methodologies described in Molecular Cloning: A Laboratory Manual (Sambrook et al., 4^{th} ed., Cold Spring Harbor Laboratory Press, Cold Spring Harbor, N.Y., 2012); Current Protocols in Molecular Biology (F. M. Ausubel, et al. eds., 2003); the series Methods in Enzymology (Academic Press, Inc.); PCR 2: A Practical Approach (M. J. MacPherson, B. D. Hames and G. R. Taylor eds., 1995); Antibodies, A Laboratory Manual (Harlow and Lane, eds., 1988); Culture of Animal Cells: A Manual of Basic Technique and Specialized Applications (R. I. Freshney, 6^{th} ed., J. Wiley and Sons, 2010); Oligonucleotide Synthesis (M. J. Gait, ed., 1984); Methods in Molecular Biology, Humana Press; Cell Biology: A Laboratory Notebook (J. E. Cellis, ed., Academic Press, 1998); Introduction to Cell and Tissue Culture (J. P. Mather and P. E. Roberts, Plenum Press, 1998); Cell and Tissue Culture: Laboratory Procedures (A. Doyle, J. B. Griffiths, and D. G. Newell, eds., J. Wiley and Sons, 1993-8); Handbook of Experimental Immunology (D. M. Weir and C. C. Blackwell, eds., 1996); Gene Transfer Vectors for Mammalian Cells (J. M. Miller and M. P. Calos, eds., 1987); PCR: The Polymerase Chain Reaction, (Mullis et al., eds., 1994); Current Protocols in Immunology (J. E. Coligan et al., eds., 1991); Short Protocols in Molecular Biology (Ausubel et al., eds., J. Wiley and Sons, 2002); Immunobiology (C. A. Janeway et al., 2004); Antibodies (P. Finch, 1997); Antibodies: A Practical Approach (D. Catty., ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane, Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al., eds., J. B. Lippincott Company, 2011).

### II. Definitions

For purposes of interpreting this specification, the following definitions will apply and whenever appropriate, terms used in the singular will also include the plural and vice versa. In the event that any definition set forth below conflicts with any document incorporated herein by reference, the definition set forth herein shall control. Additional definitions are set forth throughout the detailed description.

As used herein, the singular form term “a,” “an,” and “the” entity refers to one or more of that entity unless indicated otherwise. As such, the terms “a” (or “an” or “the”), “one or more,” and “at least one” can be used interchangeably herein.

It is understood that aspects and aspects of the disclosure described herein include “comprising,” “consisting,” and “consisting essentially of” aspects and aspects. It is also understood that wherever aspects and aspects are described herein with the language “comprising,” otherwise analogous aspects or aspects described in terms of “consisting of” and/or “consisting essentially of” are also provided.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B, B or C; A and C; A and B; B and C; A (alone); B (alone); and C (alone).

For all compositions described herein, and all methods using a composition described herein, the compositions can either comprise the listed components or steps, or can “consist essentially of” the listed components or steps. When a composition is described as “consisting essentially of” the listed components, the composition contains the components listed, and can further contain other components which do not substantially affect the methods disclosed, but do not contain any other components which substantially affect the methods disclosed other than those components expressly listed; or, if the composition does contain extra components other than those listed which substantially affect the methods disclosed, the composition does not contain a sufficient concentration or amount of the extra components to substantially affect the methods disclosed. When a method is described as “consisting essentially of” the listed steps, the method contains the steps listed, and can further contain other steps that do not substantially affect the methods disclosed, but the method does not contain any other steps which substantially affect the methods disclosed other than those steps expressly listed. As a non-limiting specific example, when a composition is described as “consisting essentially of” a component, the composition can additionally contain any amount of pharmaceutically acceptable carriers, vehicles, or diluents and other such components which do not substantially affect the methods disclosed.

Units, prefixes, and symbols are denoted in their Système International de Unites (SI) accepted form. Numeric ranges are inclusive of the numbers defining the range. The headings provided herein are not limitations of the various aspects of the disclosure, which can be had by reference to the specification as a whole. Accordingly, the terms defined immediately below are more fully defined by reference to the specification in its entirety.

The term “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower).

As used herein, the term “antigen” refers to any natural or synthetic immunogenic substance (i.e., can induce an immune response in vitro and/or in vivo), such as a protein, peptide, or hapten. Non-limiting examples of antigens are provided elsewhere in the present disclosure.

As used herein, the term “associated with” refers to a close relationship between two or more entities or properties. For instance, when used to describe a disease or condition, the term “associated with” refers to an increased likelihood that a subject suffers from the disease or condition when the subject exhibits an abnormal expression of the protein and/or gene (e.g., KRAS mutant). In some aspects, the abnormal expression of the protein and/or gene causes the disease or condition. In some aspects, the abnormal expression does not necessarily cause but is correlated with the disease or condition.

As used herein, the term “epitope” refers to the part of a protein that can be recognized by the immune system (e.g., by antibodies, B cells, and/or T cells) and thereby, induce an immune response.

As used herein the term “linked” refers to a covalent or non-covalent bond formed between a first moiety and a second moiety, e.g., first coding region and a second coding region. As further described herein, in some aspects, the first and second moieties can be linked with a linker.

The term “constriction” as used herein refers to a narrowed passageway. In some aspects, the constriction is a microfluidic channel, such as that contained within a microfluidic device. In some aspects, the constriction is a pore or contained within a pore. Where the constriction is a pore, in some aspects, the pore is contained in a surface. Unless indicated otherwise, the term constriction refers to both microfluidic channels and pores, as well as other suitable constrictions available in the art. Therefore, where applicable, disclosures relating to microfluidic channels can also apply to pores and/or other suitable constrictions available in the art. Similarly, where applicable, disclosures relating to pores can equally apply to microfluidic channels and/or other suitable constrictions available in the art.

The term “pore” as used herein refers to an opening, including without limitation, a hole, tear, cavity, aperture, break, gap, or perforation within a material. In some aspects, (where indicated) the term refers to a pore within a surface of a microfluidic device, such as those described in the present disclosure. In some aspects, (where indicated) a pore can refer to a pore in a cell wall and/or cell membrane.

The term “membrane” as used herein refers to a selective barrier or sheet containing pores. The term includes, but is not limited to, a pliable sheet-like structure that acts as a boundary or lining. In some aspects, the term refers to a surface or filter containing pores. This term is distinct from the term “cell membrane,” which refers to a semipermeable membrane surrounding the cytoplasm of cells.

The term “filter” as used herein refers to a porous article that allows selective passage through the pores. In some aspects, the term refers to a surface or membrane containing pores.

As used herein, the terms “deform” and “deformity” (including derivatives thereof) refer to a physical change in a cell. As described herein, as a cell passes through a constriction (such as those of the present disclosure), it experiences various forces due to the constraining physical environment, including but not limited to mechanical deforming forces and/or shear forces that causes perturbations in the cell membrane. As used herein, a “perturbation” within the cell membrane refers to any opening in the cell membrane that is not present under normal steady state conditions (e.g., no deformation force applied to the cells). Perturbation can comprise a hole, tear, cavity, aperture, pore, break, gap, perforation, or combinations thereof.

The term “polynucleotide” or “nucleic acid” as used herein refers to a polymeric form of nucleotides of any length, either ribonucleotides or deoxyribonucleotides. Thus, this term includes, but is not limited to, single-, double- or multi-stranded DNA or RNA, genomic DNA, cDNA, DNA-RNA hybrids, or a polymer comprising purine and pyrimidine bases, or other natural, chemically or biochemically modified, non-natural, or derivatized nucleotide bases. The backbone of the polynucleotide can comprise sugars and phosphate groups (as can typically be found in RNA or DNA), or modified or substituted sugar or phosphate groups. Alternatively, the backbone of the polynucleotide can comprise a polymer of synthetic subunits such as phosphoramidates and thus can be an oligodeoxynucleoside phosphoramidate (P-NH2) or a mixed phosphoramidate-phosphodiester oligomer. In addition, a double-stranded polynucleotide can be obtained from the single stranded polynucleotide product of chemical synthesis either by synthesizing the complementary strand and annealing the strands under appropriate conditions, or by synthesizing the complementary strand de novo using a DNA polymerase with an appropriate primer. As described herein, a nucleic acid that can be delivered to a cell using the squeeze processing methods provided herein comprises a RNA (e.g., mRNA). As used herein, “RNA” comprises both self-amplifying RNA (e.g., self-amplifying mRNA) and non-self-amplifying RNA (e.g., non-self-amplifying mRNA). As used herein, the term “self-amplifying RNA” refers to a RNA molecule that can replicate in a host, resulting in an increase in the amount of RNA and proteins encoded by the RNA (e.g., antigens). As used herein, the term “mRNA” refers to any polynucleotides (either self-amplifying or non-self-amplifying) which encodes at least one polypeptide.

The terms “polypeptide” and “protein” are used interchangeably to refer to a polymer of amino acid residues, and are not limited to a minimum length. Such polymers of amino acid residues can contain natural or non-natural amino acid residues, and include, but are not limited to, peptides, oligopeptides, dimers, trimers, and multimers of amino acid residues. Both full-length proteins and fragments thereof are encompassed by the definition. The terms also include post-expression modifications of the polypeptide, for example, glycosylation, sialylation, acetylation, phosphorylation, and the like. Furthermore, for purposes of the present disclosure, a “polypeptide” refers to a protein which includes modifications, such as deletions, additions, and substitutions (generally conservative in nature), to the native sequence, as long as the protein maintains the desired activity. These modifications can be deliberate, as through site-directed mutagenesis, or can be accidental, such as through mutations of hosts which produce the proteins or errors due to PCR amplification.

An “immune response,” as used herein, refers to a biological response within a vertebrate against foreign agents or abnormal, e.g., cancerous cells, which response protects the organism against these agents and diseases caused by them. An immune response is mediated by the action of one or more cells of the immune system (for example, a T lymphocyte, B lymphocyte, natural killer (NK) cell, macrophage, eosinophil, mast cell, dendritic cell or neutrophil) and soluble macromolecules produced by any of these cells or the liver (including antibodies, cytokines, and complement) that results in selective targeting, binding to, damage to, destruction of, and/or elimination from the vertebrate's body of invading pathogens, cells or tissues infected with pathogens, cancerous or other abnormal cells, or, in cases of autoimmunity or pathological inflammation, normal human cells or tissues. An immune reaction includes, e.g., activation or inhibition of a T cell, e.g., an effector T cell, a Th cell, a CD4+ cell, a CD8+ T cell, or a Treg cell, or activation or inhibition of any other cell of the immune system, e.g., NK cell. Accordingly, an immune response can comprise a humoral immune response (e.g., mediated by B-cells), cellular immune response (e.g., mediated by T cells), or both humoral and cellular immune responses. In some aspects, an immune response that is induced by the polynucleotides described herein comprises a T cell response. In some aspects, the T cell response is mediated by CD8+ T cells. In some aspects, the T cell response is mediated by CD4+ T cells. In some aspects, the T cell response is mediated by both CD8+ and CD4+ T cells.

### III. Polynucleotides

The present disclosure is directed to polynucleotides (e.g., isolated polynucleotides) comprising multiple nucleotide sequences (e.g., at least two, at least three, at least four, at least five, at least six, at least seven, at least eight, at least nine, or at least ten), wherein each of the multiple nucleotide sequences encode an antigen. Nucleotide sequences encoding an antigen is also referred to herein as a “coding region.” As demonstrated and further described herein, the polynucleotides of the present disclosure differ (in structure and/or function) as compared to corresponding polynucleotides that naturally exists in nature.

### Coding Regions:

In some aspects, a polynucleotide described herein comprises coding regions from multiple proteins in a single open reading frame (ORF), wherein the coding regions do not naturally exist together (e.g., within a single polynucleotide) in nature. For instance, in some aspects, a polynucleotide of the present disclosure comprises a single ORF with a first coding region and a second coding region, wherein the first coding region can encode a cancer antigen and the second coding region can encode a different cancer antigen. For instance, in some aspects, a polynucleotide of the present disclosure comprises a first coding region and a second coding region, wherein the first coding region can encode a viral antigen and the second coding region can encode a different viral antigen. For instance, in some aspects, a polynucleotide of the present disclosure comprises a single ORF first coding region and a second coding region, wherein the first coding region can encode a cancer antigen (e.g., KRAS antigen) and the second coding region can encode a viral antigen (e.g., HPV, HIV, or HBV). Non-limiting examples of antigens that are useful for the present disclosure are provided elsewhere in the present disclosure. As will be apparent to those skilled in the arts, such polynucleotides can be particularly useful in simultaneously combating many diverse pathogens and/or diseases.

In some aspects, a polynucleotide described herein comprises coding regions, wherein the coding regions can naturally exist together within a single polynucleotide (e.g., within the same ORF). For instance, in some aspects, the coding regions comprises a first coding region and a second coding region, wherein the first coding region can encode a first epitope of a protein (e.g., viral protein) and the second coding region can encode a second epitope of the same protein, wherein the protein is encoded in nature by a single ORF of a polynucleotide. Where a polynucleotide useful for the present disclosure comprises such coding regions, in some aspects, the coding regions are arranged within the ORF of the polynucleotide in a specific order, wherein the specific order does not naturally exist in nature.

To help illustrate, an exemplary polynucleotide that exists in nature comprises a single ORF with a first coding region, a second coding region, and a third coding region, wherein the first, second, and third coding regions are arranged as follows (from 5′ to 3′): (first coding region), (second coding region), and (third coding region). Where a polynucleotide described herein comprises such coding regions, in some aspects, the first coding region, the second coding region, and the third coding region are arranged within the single ORF of the polynucleotide in any of the following order (from 5′ to 3′):
(a) (first coding region), (third coding region), and (second coding region);
(b) (second coding region), (first coding region), and (third coding region);
(c) (second coding region), (third coding region), and (first coding region);
(d) (third coding region), (first coding region), and (second coding region); or
(c) (third coding region), (second doing region), and (first coding region).

Additional structural features of the polynucleotides are provided throughout the present disclosure. For instance, in some aspects, a polynucleotide described comprises a single ORF with multiple coding regions (e.g., a first coding region and a second coding region), wherein one or more of the coding regions encode a full-length protein. To illustrate, in some aspects, a polynucleotide comprises a single ORF with a first coding region and a second coding region, wherein the first coding region encodes the full-length protein of a HPV E6 protein (e.g., HPV-16 or HPV-18) and the second coding region encodes the full-length protein of a HPV E7 protein (e.g., HPV-16 or HPV-18). However, as demonstrated herein, it is not necessary for a coding region to encode a full length protein (or a large fragment thereof) to exert a therapeutic effect (e.g., inducing an immune response against the protein). Accordingly, the polynucleotides described herein comprise a single ORF comprising multiple coding regions, wherein one or more of the antigens encoded by the multiple coding regions comprise a peptide fragment of a larger protein. In some aspects, the peptide fragment is less than about 99%, less than about 98%, less than about 97%, less than about 96%, less than about 95%, less than about 90%, less than about 85%, less than about 80%, less than about 75%, less than about 70%, less than about 65%, less than about 60%, less than about 55%, less than about 50%, less than about 45%, less than about 40%, less than about 35%, less than about 30%, less than about 25%, less than about 20%, less than about 15%, less than about 10%, or less than about 5% in length as compared to the corresponding full-length fragment. In some aspects, an antigen encoded by one or more of the multiple coding regions less than about 200 amino acids in length. In some aspects, the antigen is less than about 150 amino acids in length. In some aspects, the antigen less than about 100 amino acids in length. In some aspects, the antigen is less than about 50 amino acids in length. In some aspects, an antigen encoded by one or more of the multiple coding regions present in a single ORF of a polynucleotide described herein is about 5 amino acids to about 200 amino acids in length. In some aspects, the antigen is about 5 amino acids to about 150 amino acids in length. In some aspects, the antigen is about 5 amino acids to about 100 amino acids in length. In some aspects, the antigen is about 5 amino acids to about 50 amino acids in length. In some aspects, the antigen is about 5 amino acids, about 6 amino acids, about 7 amino acids, about 8 amino acids, about 9 amino acids, about 10 amino acids, about 11 amino acids, about 12 amino acids, about 13 amino acids, about 14 amino acids, about 15 amino acids, about 16 amino acids, about 17 amino acids, about 18 amino acids, about 19 amino acids, about 20 amino acids, about 21 amino acids, about 22 amino acids, about 23 amino acids, about 24 amino acids, about 25 amino acids, about 26 amino acids, about 27 amino acids, about 28 amino acids, about 29 amino acids, about 30 amino acids, about 31 amino acids, about 32 amino acids, about 33 amino acids, about 34 amino acids, about 35 amino acids, about 36 amino acids, about 37 amino acids, about 38 amino acids, about 39 amino acids, about 40 amino acids, about 41 amino acids, about 42 amino acids, about 43 amino acids, about 44 amino acids, about 45 amino acids, about 46 amino acids, about 47 amino acids, about 48 amino acids, about 49 amino acids, or about 50 amino acids in length. In some aspects, the antigen is about 10 to about 50 amino acids in length. In some aspects, the antigen is about 10 to about 40 amino acids in length. In some aspects, the antigen is about 10 to about 30 amino acids in length. In some aspects, the antigen is about 10 to about 20 amino acids in length. In some aspects, the antigen is about 20 to about 50 amino acids in length. In some aspects, the antigen is about 20 to about 40 amino acids in length. In some aspects, the antigen is about 20 to about 30 amino acids in length. In some aspects, the antigen is about 30 to about 50 amino acids in length. In some aspects, the antigen is about 30 to 40 amino acids in length. In some aspects, the antigen is about 40 to about 50 amino acids in length. In some aspects, the antigen is about 8 to about 11 amino acids in length. In some aspects, the antigen is about 10 to about 17 amino acids in length. In some aspects, the antigen is about 10 amino acids in length. In some aspects, the antigen is about 15 amino acids in length. In some aspects, the antigen is about 20 amino acids in length. In some aspects, the antigen is about 25 amino acids in length. In some aspects, the antigen is about 30 amino acids in length. In some aspects, the antigen is about 35 amino acids in length. In some aspects, the antigen is about 40 amino acids in length. In some aspects, the antigen is about 45 amino acids in length. In some aspects, the antigen is about 50 amino acids in length. In some aspects, the antigen is about 8 amino acids in length.

Not to be bound by any one theory, because of the size of the antigens, polynucleotides described herein can be designed to comprise many coding regions. In some aspects, a polynucleotide described comprises a single ORF with about two coding regions, about three coding regions, about four coding regions, about five coding regions, about six coding regions, about seven coding regions, about eight coding regions, about nine coding regions, about 10 coding regions, about 11 coding regions, about 12 coding regions, about 13 coding regions, about 14 coding regions, about 15 coding regions, about 16 coding regions, about 17 coding regions, about 18 coding regions, about 19 coding regions, about 20 coding regions, about 21 coding regions, about 22 coding regions, about 23 coding regions, about 24 coding regions, about 25 coding regions, about 26 coding regions, about 27 coding regions, about 28 coding regions, about 29 coding regions, or about 30 coding regions. As described herein, in some aspects, two or more of the coding regions are linked. In some aspects, each of the coding regions are linked. In some aspects, a polynucleotide described herein comprises a single ORF with two linked coding regions. In some aspects, a polynucleotide comprises a single ORF with three linked coding regions. In some aspects, a polynucleotide comprises a single ORF with four linked coding regions. In some aspects, a polynucleotide described herein comprises a single ORF with five linked coding regions. In some aspects, a polynucleotide described herein comprises a single ORF with six linked coding regions. In some aspects, a polynucleotide comprises a single ORF with seven linked coding regions. In some aspects, a polynucleotide comprises a single ORF with eight linked coding regions. In some aspects, a polynucleotide comprises a single ORF with nine linked coding regions. In some aspects, a polynucleotide comprises a single ORF with ten linked coding regions. In some aspects, a polynucleotide comprises a single ORF with 11 linked coding regions. In some aspects, a polynucleotide comprises a single ORF with 12 linked coding regions. In some aspects, a polynucleotide comprises a single ORF with 13 linked coding regions. In some aspects, a polynucleotide comprises a single ORF with 14 linked coding region. In some aspects, a polynucleotide comprises a single ORF with 15 linked coding regions. In some aspects, a polynucleotide comprises a single ORF with 16 linked coding regions. In some aspects, a polynucleotide comprises a single ORF with 17 linked coding regions. In some aspects, a polynucleotide comprises a single ORF with 18 linked coding regions. In some aspects, a polynucleotide comprises a single ORF with 19 linked coding regions. In some aspects, a polynucleotide comprises a single ORF with 20 linked coding regions. In some aspects, a polynucleotide comprises a single ORF with 21 linked coding regions. In some aspects, a polynucleotide comprises a single ORF with 22 linked coding regions. In some aspects, a polynucleotide comprises a single ORF with 23 linked coding regions. In some aspects, a polynucleotide comprises a single ORF with 24 linked coding regions. In some aspects, a polynucleotide comprises a single ORF with 25 linked coding regions. In some aspects, a polynucleotide comprises a single ORF with 26 linked coding regions. In some aspects, a polynucleotide comprises a single ORF with 27 linked coding regions. In some aspects, a polynucleotide comprises a single ORF with 28 linked coding regions. In some aspects, a polynucleotide comprises a single ORF with 29 linked coding regions. In some aspects, a polynucleotide comprises a single ORF with 30 linked coding regions. In some aspects, each of the coding regions present in a single ORF of a polynucleotide described herein encodes a different antigen. In some aspects, at least two of the coding regions encode a different antigen.

In some aspects, any of the polynucleotides described herein can be used in combination. For instance, in some aspects, a composition can comprise a first polynucleotide and a second polynucleotide, wherein the first polynucleotide comprises a single ORF with multiple (e.g., about 10) linked coding regions and the second polynucleotide comprises a single ORF with multiple (e.g., about 10) linked coding regions. In some aspects, the multiple linked coding regions of the first polynucleotide and the multiple linked coding regions of the second polynucleotide encode different antigens. To illustrate, in some aspects, the multiple coding regions of the first polynucleotide can encode tumor antigens (e.g., neoantigens) and the multiple coding regions of the second polynucleotide can encode non-self antigens (e.g., derived from a virus). Such first and second polynucleotides can be administered to a subject in combination as a single composition. In some aspects, the first and second polynucleotides can be used in combination but as separate compositions.

Accordingly, some aspects of the present disclosure relates to a polynucleotide (e.g., isolated polynucleotide) comprising a single ORF with a first coding region encoding a first antigen, a second coding region encoding a second antigen, and a third coding region encoding a third antigen, and wherein the first coding region, the second coding region, and the third coding region are linked. In some aspects, at least two of the encoded linked antigens are not the same. In some aspects, each of the encoded linked antigens are different. In some aspects, at least two of the encoded linked antigens are the same. In some aspects, the first coding region, the second coding region, and the third coding region are arranged in an order, wherein the order is different as compared to a corresponding order present in a reference polynucleotide, wherein the reference polynucleotide comprises a naturally-existing corresponding polynucleotide. In some aspects, the particular order of the coding regions within the single ORF of a polynucleotide can help improve one or more properties of the polynucleotide (e.g., improved translation when delivered to a cell). For instance, to help illustrate, in some aspects, a first polynucleotide comprises a single ORF comprising a first coding region encoding an antigen, a second coding region encoding an antigen, and a third coding region encoding an antigen, wherein the first, second, and third coding regions are arranged within the ORF as follows (from 5′ to 3′): (first coding region)-(second coding region)-(third coding region). In some aspects, a second polynucleotide comprises a single ORF with the same first, second, and third coding regions of the first polynucleotide, but wherein the first, second, and third coding regions are arranged within the ORF as follows (from 5′ to 3′): (first coding region)-(third coding region)-(second coding region). In some aspects, a third polynucleotide comprises a single ORF with the same first, second, and third coding regions, but wherein the coding regions are arranged as follows (from 5′ to 3′): (second coding region)-(third coding region)-(first coding region). For each of the first, second, and third polynucleotides, the three coding regions can be linked with a linker and/or without a linker (e.g., directly conjugated via natural peptide bond). Not to be bound by any one theory, in some aspects, each of the first, second, and third polynucleotides can be associated with different therapeutic effects. For instance, in some aspects, when the first, second, and/or polynucleotides are delivered into a cell (e.g., using squeeze processing described herein), they can result in different translation efficiency (e.g., cells modified to comprise the second polynucleotide could have higher encoded protein expression compared to cells modified to comprise the first or second polynucleotide). Accordingly, in some aspects, a polynucleotide described herein can have improved effects (e.g., better translation) as compared to a corresponding polynucleotide with the same coding regions but arranged in a different order.

In some aspects, a polynucleotide described herein comprises a single ORF with a first coding region encoding a first antigen, a second coding region encoding a second antigen, a third coding region encoding a third antigen, and a fourth coding region encoding a fourth antigen, and wherein the first coding region, the second coding region, the third coding region, and the fourth coding region are linked. In some aspects, the first coding region, the second coding region, the third coding region, and the fourth coding region are arranged in an order, wherein the order is different as compared to a corresponding order present in a reference polynucleotide, wherein the reference polynucleotide comprises a naturally-existing corresponding polynucleotide. In some aspects, at least two of the encoded linked antigens are not the same. In some aspects, each of the encoded linked antigens are different. In some aspects, at least two of the encoded linked antigens are the same.

In some aspects, a polynucleotide described herein comprises a single ORF with a first coding region encoding a first antigen, a second coding region encoding a second antigen, a third coding region encoding a third antigen, a fourth coding region encoding a fourth antigen, and a fifth coding region encoding a fifth antigen, and wherein the first coding region, the second coding region, the third coding region, the fourth coding region, and the fifth coding region are linked. In some aspects, the first coding region, the second coding region, the third coding region, the fourth coding region, and the fifth coding region are arranged in an order, wherein the order is different as compared to a corresponding order present in a reference polynucleotide, wherein the reference polynucleotide comprises a naturally-existing corresponding polynucleotide. In some aspects, at least two of the encoded linked antigens are not the same. In some aspects, each of the encoded linked antigens are different. In some aspects, at least two of the encoded linked antigens are the same.

In some aspects, a polynucleotide described herein comprises a single ORF with a first coding region encoding a first antigen, a second coding region encoding a second antigen, a third coding region encoding a third antigen, a fourth coding region encoding a fourth antigen, a fifth coding region encoding a fifth antigen, and a sixth coding region encoding a sixth antigen, and wherein the first coding region, the second coding region, the third coding region, the fourth coding region, the fifth coding region, and the sixth coding region are linked. In some aspects, the first coding region, the second coding region, the third coding region, the fourth coding region, the fifth coding region, and the sixth coding region are arranged in an order, wherein the order is different as compared to a corresponding order present in a reference polynucleotide, wherein the reference polynucleotide comprises a naturally-existing corresponding polynucleotide. In some aspects, at least two of the encoded linked antigens are not the same. In some aspects, each of the encoded linked antigens are different. In some aspects, at least two of the encoded linked antigens are the same.

In some aspects, a polynucleotide described herein comprises a single ORF with a first coding region encoding a first antigen, a second coding region encoding a second antigen, a third coding region encoding a third antigen, a fourth coding region encoding a fourth antigen, a fifth coding region encoding a fifth antigen, a sixth coding region encoding a sixth antigen, and a seventh coding region encoding a seventh antigen, and wherein the first coding region, the second coding region, the third coding region, the fourth coding region, the fifth coding region, the sixth coding region, and the seventh coding region are linked. In some aspects, the first coding region, the second coding region, the third coding region, the fourth coding region, the fifth coding region, the sixth coding region, and the seventh coding region are arranged in an order, wherein the order is different as compared to a corresponding order present in a reference polynucleotide, wherein the reference polynucleotide comprises a naturally-existing corresponding polynucleotide. In some aspects, at least two of the encoded linked antigens are not the same. In some aspects, each of the encoded linked antigens are different. In some aspects, at least two of the encoded linked antigens are the same.

In some aspects, a polynucleotide described herein comprises a single ORF with a first coding region encoding a first antigen, a second coding region encoding a second antigen, a third coding region encoding a third antigen, a fourth coding region encoding a fourth antigen, a fifth coding region encoding a fifth antigen, a sixth coding region encoding a sixth antigen, a seventh coding region encoding a seventh antigen, and an eighth coding region encoding an eighth antigen, and wherein the first coding region, the second coding region, the third coding region, the fourth coding region, the fifth coding region, the sixth coding region, the seventh coding region, and the eighth coding region are linked. In some aspects, the first coding region, the second coding region, the third coding region, the fourth coding region, the fifth coding region, the sixth coding region, the seventh coding region, and the eighth coding region are arranged in an order, wherein the order is different as compared to a corresponding order present in a reference polynucleotide, wherein the reference polynucleotide comprises a naturally-existing corresponding polynucleotide. In some aspects, at least two of the encoded linked antigens are not the same. In some aspects, each of the encoded linked antigens are different. In some aspects, at least two of the encoded linked antigens are the same.

In some aspects, a polynucleotide described herein comprises a single ORF with a first coding region encoding a first antigen, a second coding region encoding a second antigen, a third coding region encoding a third antigen, a fourth coding region encoding a fourth antigen, a fifth coding region encoding a fifth antigen, a sixth coding region encoding a sixth antigen, a seventh coding region encoding a seventh antigen, an eighth coding region encoding an eighth antigen, and a ninth coding region encoding a ninth antigen, and wherein the first coding region, the second coding region, the third coding region, the fourth coding region, the fifth coding region, the sixth coding region, the seventh coding region, the eighth coding region, and the ninth coding region are linked. In some aspects, the first coding region, the second coding region, the third coding region, the fourth coding region, the fifth coding region, the sixth coding region, the seventh coding region, the eighth coding region, and the ninth coding region are arranged in an order, wherein the order is different as compared to a corresponding order present in a reference polynucleotide, wherein the reference polynucleotide comprises a naturally-existing corresponding polynucleotide. In some aspects, at least two of the encoded linked antigens are not the same. In some aspects, each of the encoded linked antigens are different. In some aspects, at least two of the encoded linked antigens are the same.

In some aspects, a polynucleotide described herein comprises a single ORF with a first coding region encoding a first antigen, a second coding region encoding a second antigen, a third coding region encoding a third antigen, a fourth coding region encoding a fourth antigen, a fifth coding region encoding a fifth antigen, a sixth coding region encoding a sixth antigen, a seventh coding region encoding a seventh antigen, an eighth coding region encoding an eighth antigen, a ninth coding region encoding a ninth antigen, and a tenth coding region encoding a tenth antigen, and wherein the first coding region, the second coding region, the third coding region, the fourth coding region, the fifth coding region, the sixth coding region, the seventh coding region, the eighth coding region, the ninth coding region, and the tenth coding region are linked. In some aspects, the first coding region, the second coding region, the third coding region, the fourth coding region, the fifth coding region, the sixth coding region, the seventh coding region, the eighth coding region, the ninth coding region, and the tenth coding region are arranged in an order, wherein the order is different as compared to a corresponding order present in a reference polynucleotide, wherein the reference polynucleotide comprises a naturally-existing corresponding polynucleotide. In some aspects, at least two of the encoded linked antigens are not the same. In some aspects, each of the encoded linked antigens are different. In some aspects, at least two of the encoded linked antigens are the same.

### Antigens:

As described herein, polynucleotides described herein comprises a single ORF with coding regions from multiple proteins, wherein the coding regions encode an antigen. In some aspects, the antigen can comprise an epitope. In some aspects, the antigen can comprise an immunogenic epitope. Accordingly, in some aspects, one or more of the multiple coding regions can encode distinct (i.e., different) epitopes of a single larger protein, wherein the single larger protein comprises multiple distinct epitopes. For instance, in some aspects, a polynucleotide described herein comprises a single ORF with at least a first coding region and a second coding region, and wherein the first coding region and the second coding region encode for distinct epitopes of the same protein. In some aspects, one or more of the multiple coding regions can encode epitopes from different proteins. For instance, in some aspects, a polynucleotide of the present disclosure comprises a single ORF with at least a first coding region and a second coding region, wherein the first coding region encodes for an epitope of a first protein, wherein the second coding region encodes for an epitope of a second protein, and wherein the first protein and the second protein are not the same (e.g., KRAS protein and HPV protein, respectively). In some aspects, an antigen comprises a wild-type protein. In some aspects, an antigen comprises a peptide fragment derived from the wild-type protein. In some aspects, an antigen comprises a protein with at least one or more amino acid modifications (e.g., substitutions, deletions, additions, or indels) as compared to the corresponding wild-type protein (also referred to herein as a “mutant protein”) (e.g., KRAS mutant). In some aspects, an antigen comprises a peptide fragment derived from such a mutant protein.

Non-limiting examples of antigens that can be used with the present disclosure include a cancer antigen, non-self antigen, self-antigen associated with a tumor, disease-associated antigen, or combinations thereof.

In some aspects, an antigen comprises a cancer antigen. As used herein, the terms “cancer antigen” and “tumor antigen” (and derivatives thereof) can be used interchangeably and refer to any antigen that are common to specific hyperproliferative disorders, such as cancer. Non-limiting examples of cancer antigens include: guanylate cyclase C (GC-C), epidermal growth factor receptor (EGFR or erbB-1), human epidermal growth factor receptor 2 (HER2 or erbB2), erbB-3, erbB-4, MUC-1, melanoma-associated chondroitin sulfate proteoglycan (MCSP), mesothelin (MSLN), folate receptor 1 (FOLR1), CD4, CD19, CD20, CD22, CD30, CD33, CD38, CD44, CD44v6, CD44v7/8, CD70, CD123, CD138, CD171, CEA, CSPG4, CXCR5, c-Met, HERV-envelope protein, eriostin, Bigh3, SPARC, BCR, CD79, CD37, EGFRvIII, EGP2, EGP40, IGFr, LICAM, AXL, Tissue Factor (TF), CD74, EpCAM, EphA2, MRP3cadherin 19 (CDH19), epidermal growth factor 2 (HER2), 5T4, 8H9, αᵥβ₆ integrin, BCMA, B7-H3, B7-H6, CAIX, CA9, FAP, FBP, fetal AchR, FRcc, GD2, GD3, Glypican-1 (GPC1), Glypican-2 (GPC2), Glypican-3 (GPC3), MAGE1, MAGEA10, NY-ESO-1, IL-13Rcc2, Lewis-Y, KDR, MCSP, Mesothelin, Muc1, Muc16, NCAM, NKG2D ligands, PRAME, PSC1, PSCA, PSMA, ROR1, ROR2, SP17, survivin, TAG72, TEMs, carcinoembryonic antigen, HMW-MAA, VEGF, CLDN18.2, neoantigen, KRAS, or combinations thereof. In some aspects, the cancer antigen comprises KRAS.

Kirsten rat sarcoma viral oncogene homology (KRAS) is a member of a superfamily of guanosine-5-triphosphatase (GTPase) proteins that also includes NRAS and HRAS. The primary role of the members of this superfamily is to transmit signals from upstream cell surface receptors (e.g., EGFR, FGFR, and ERBB2-4) to downstream proliferation and survival pathways such as RAF-MEK-ERK, PI3K-AKT-mTOR, and RALGDS-RA. Adderley, H., et al., EBioMedicine 41:711-716 (2019). KRAS mutations have been implicated in many types of cancers, including more than 90% of pancreatic cancers, 35-45% of colorectal cancers, and approximately 25% of lung cancers. Zeitouni, D., et al., Cancers 8 (4): 45 (2016); Tan, C., et al., World J Gastroenterol 18 (37): 5171-5180 (2012); and Roman, M., et al., Molecular Cancer 17:33 (2018). KRAS mutations have also been associated with very poor prognosis (e.g., 5 year survival rate of about 9% in pancreatic cancer), and many patients with the KRAS mutations are resistant to various cancer therapies. Del Re, M., et al., Oncotarget 9 (5): 6630-6643 (2017). Accordingly, there is a need for new and improved treatment options for cancers associated with KRAS mutations.

KRAS is known in the art by various names. Such names include: KRAS Proto-Oncogene, GTPase; V-Ki-Ras2 Kirsten Rat Sarcoma 2 Viral Oncogene Homolog; GTPase KRas; C-Ki-Ras; K-Ras2; KRAS2; RASK2; V-Ki-Ras2 Kirsten Rat Sarcoma Viral Oncogene Homolog; Kirsten Rat Sarcoma Viral Proto-Oncogene; Cellular Transforming Proto-Oncogene; Cellular C-Ki-Ras2 Proto-Oncogene; Transforming Protein P21; PR310 C-K-Ras Oncogene, C-Kirsten-Ras Protein; K-Ras P21 Protein; and Oncogene KRAS2.

There are two isoforms of the human wild-type KRAS protein (P01116), resulting from alternative splicing. Isoform 2A (Accession Number: P01116-1; SEQ ID NO: 16) is the canonical sequence. It is also known as K-Ras4A. Isoform 2B (Accession Number: P01116-2; also known as K-Ras4B; SEQ ID NO: 17) differs from the canonical sequence as follows: (i) 151-153: RVE→GVD; and (ii) 165-189: QYRLKKISKEEKTPGCVKIKKCIIM→KHKEKMSKDGKKKKKKSKTKCVIM.

Natural variants of the human KRAS gene product are known. For example, natural variants of human KRAS protein can contain one or more amino acid substitutions selected from: K5E, K5N, G10GG, G10V, G12A, G12C, G12D, G12F, G12I, G12L, G12R, G12S, G12V, G13C, G13D, G13E, G13R, G13V, V14I, L19F, T20M, Q22E, Q22H, Q22K, Q22R, Q25H, N26Y, F28L, E31K, D33E, P34L, P34Q, P34R, 136M, R41K, D57N, T58I, A59T, G60D, G60R, G60S, G60V, Q61A, Q61H, Q61K, Q61L, Q61P, Q61R, E63K, S65N, R68S, Y71H, T74A, L79I, R97I, Q99E, M111L, K117N, K117R, D119G, S122F, T144P, A146P, A146T, A146V, K147E, K147T, R149K, L159S, 1163S, R164Q, 1183N, 184M, or combinations thereof. Natural variants that are specific to KRAS protein Isoform 2B contain one or more amino acid substitutions selected from: V152G, D153V, F156I, F156L, or combinations thereof.

As is apparent from the above disclosure, in some aspects, one or more of the coding regions of a polynucleotide described herein encodes for a KRAS antigen, wherein the KRAS antigen comprises an amino acid sequence which differs in sequence as compared to the amino acid sequence of a corresponding wild-type KRAS antigen. Such KRAS antigens are also referred to herein as “KRAS mutant” or “KRAS variant.” In some aspects, the amino acid sequence of the KRAS mutant has a sequence identity of less than about 99%, less than about 98%, less than about 97%, less than about 96%, less than about 95%, less than about 94%, less than about 93%, less than about 92%, less than about 91%, less than about 90%, less than about 85%, or less than about 90% as compared to the amino acid sequence of the corresponding wild-type KRAS antigen. In some aspects, the amino acid sequence of the KRAS mutant comprises one or more amino acid substitutions, wherein the one or more amino acid substitutions are associated with a cancer. In some aspects, the amino acid sequence of the KRAS mutant comprises any of the amino acid substitutions described herein. For instance, in some aspects, a polynucleotide described herein comprises a single ORF with multiple coding regions, wherein the multiple coding regions are linked, wherein at least one of the multiple coding regions encode for a KRAS antigen, wherein the amino acid sequence of the KRAS antigen comprises the G12V amino acid substitution. In some aspects, a polynucleotide described herein comprises a single ORF with multiple coding regions, wherein the multiple coding regions are linked, wherein at least one of the multiple coding regions encode for a KRAS antigen, wherein the amino acid sequence of the KRAS antigen comprises the G12D amino acid substitution. In some aspects, a polynucleotide described herein comprises a single ORF with multiple coding regions, wherein the multiple coding regions are linked, wherein at least one of the multiple coding regions encode for a KRAS antigen, and wherein the amino acid sequence of the KRAS antigen comprises the G12C amino acid substitution. In some aspects, a polynucleotide described herein comprises a single ORF with multiple coding regions, wherein the multiple coding regions are linked, wherein at least one of the multiple coding regions encode for a KRAS antigen, and wherein the amino acid sequence of the KRAS antigen comprises the G13D amino acid substitution. In some aspects, a polynucleotide described herein comprises a single ORF with multiple coding regions, wherein the multiple coding regions are linked, wherein at least one of the multiple coding regions encode for a KRAS antigen, and wherein the amino acid sequence of the KRAS antigen comprises the G12A amino acid substitution. In some aspects, a polynucleotide described herein comprises a single ORF with multiple coding regions, wherein the multiple coding regions are linked, wherein at least one of the multiple coding regions encode for a KRAS antigen, and wherein the amino acid sequence of the KRAS antigen comprises the G12R amino acid substitution. In some aspects, a polynucleotide described herein comprises a single ORF with multiple coding regions, wherein the multiple coding regions are linked, wherein at least one of the multiple coding regions encode for a KRAS antigen, and wherein the amino acid sequence of the KRAS antigen comprises the G12S amino acid substitution. In some aspects, a polynucleotide described herein comprises a single ORF with multiple coding regions, wherein the multiple coding regions are linked, wherein the at least one of the multiple coding regions encode a KRAS antigen, and wherein the amino acid sequence of the KRAS antigen comprises one or more of the following: G12D¹⁻¹⁶, a G12D²⁻¹⁹, a G12D²⁻²², a G12D²⁻²⁹, a G12V¹⁻¹⁶, a G12V²⁻¹⁹, a G12V³⁻¹⁷, or a G12V³⁻⁴² antigen.

In some aspects, at least one of the multiple coding regions encode a KRAS antigen, wherein the amino acid sequence of the KRAS antigen has a sequence identity of at least about 80%, at least about 85%, at least about 90%, at least about 91%, at least about 92%, at last about 93%, at least about 94%, at least about 95%, at least about 96%, at least about 97%, at least about 98%, or about 99% as compared to the amino acid sequence set forth in any one of SEQ ID NOs: 1-15. In some aspects, a polynucleotide described herein comprises a single ORF with multiple coding regions, wherein the multiple coding regions are linked and encode for at least one KRAS antigen, wherein the KRAS antigen comprises, consists of: or consists essentially of the amino acid sequence set forth in SEQ ID NO: 1. In some aspects, a polynucleotide described herein comprises a single ORF with multiple coding regions, wherein the multiple coding regions are linked and encode for at least one KRAS antigen, wherein the KRAS antigen comprises, consists of, or consists essentially of the amino acid sequence set forth in SEQ ID NO: 2. In some aspects, a polynucleotide described herein comprises a single ORF with multiple coding regions, wherein the multiple coding regions are linked and encode for at least one KRAS antigen, wherein the KRAS antigen comprises, consists of, or consists essentially of the amino acid sequence set forth in SEQ ID NO: 3. In some aspects, a polynucleotide described herein comprises a single ORF with multiple coding regions, wherein the multiple coding regions are linked and encode for at least one KRAS antigen, wherein the KRAS antigen comprises, consists of, or consists essentially of the amino acid sequence set forth in SEQ ID NO: 4. In some aspects, a polynucleotide described herein comprises a single ORF with multiple coding regions, wherein the multiple coding regions are linked and encode for at least one KRAS antigen, wherein the KRAS antigen comprises, consists of, or consists essentially of the amino acid sequence set forth in SEQ ID NO: 5. In some aspects, a polynucleotide described herein comprises a single ORF with multiple coding regions, wherein the multiple coding regions are linked and encode for at least one KRAS antigen, wherein the KRAS antigen comprises, consists of, or consists essentially of the amino acid sequence set forth in SEQ ID NO: 6. In some aspects, a polynucleotide described herein comprises a single ORF with multiple coding regions, wherein the multiple coding regions are linked and encode for at least one KRAS antigen, wherein the KRAS antigen comprises, consists of, or consists essentially of the amino acid sequence set forth in SEQ ID NO: 7. In some aspects, a polynucleotide described herein comprises a single ORF with multiple coding regions, wherein the multiple coding regions are linked and encode for at least one KRAS antigen, wherein the KRAS antigen comprises, consists of, or consists essentially of the amino acid sequence set forth in SEQ ID NO: 8. In some aspects, a polynucleotide described herein comprises a single ORF with multiple coding regions, wherein the multiple coding regions are linked and encode for at least one KRAS antigen, wherein the KRAS antigen comprises, consists of, or consists essentially of the amino acid sequence set forth in SEQ ID NO: 9. In some aspects, a polynucleotide described herein comprises a single ORF with multiple coding regions, wherein the multiple coding regions are linked and encode for at least one KRAS antigen, wherein the KRAS antigen comprises, consists of, or consists essentially of the amino acid sequence set forth in SEQ ID NO: 10. In some aspects, a polynucleotide described herein comprises a single ORF with multiple coding regions, wherein the multiple coding regions are linked and encode for at least one KRAS antigen, wherein the KRAS antigen comprises, consists of, or consists essentially of the amino acid sequence set forth in SEQ ID NO: 11. In some aspects, a polynucleotide described herein comprises a single ORF with multiple coding regions, wherein the multiple coding regions are linked and encode for at least one KRAS antigen, wherein the KRAS antigen comprises, consists of, or consists essentially of the amino acid sequence set forth in SEQ ID NO: 12. In some aspects, a polynucleotide described herein comprises a single ORF with multiple coding regions, wherein the multiple coding regions are linked and encode for at least one KRAS antigen, wherein the KRAS antigen comprises, consists of, or consists essentially of the amino acid sequence set forth in SEQ ID NO: 13. In some aspects, a polynucleotide described herein comprises a single ORF with multiple coding regions, wherein the multiple coding regions are linked and encode for at least one KRAS antigen, wherein the KRAS antigen comprises, consists of, or consists essentially of the amino acid sequence set forth in SEQ ID NO: 14. In some aspects, a polynucleotide described herein comprises a single ORF with multiple coding regions, wherein the multiple coding regions are linked and encode for at least one KRAS antigen, wherein the KRAS antigen comprises, consists of, or consists essentially of the amino acid sequence set forth in SEQ ID NO: 15.

In some aspects, a polynucleotide described herein comprises a single ORF with at least a first coding region encoding a first KRAS antigen and a second coding region encoding a second KRAS antigen, wherein the first coding region and the second coding region are linked, wherein the amino acid sequence of the first KRAS antigen comprises the G12V amino acid substitution and the amino acid sequence of the second KRAS antigen comprises the G12D amino acid substitution. In some aspects, a polynucleotide described herein comprises a single ORF with multiple coding regions, wherein the multiple coding regions are linked, wherein one or more of the multiple coding regions encode for a KRAS mutant antigen (e.g., comprising the G12D and/or G12V substitution), and wherein one or more of the multiple coding regions encode for an additional tumor antigen (e.g., NY-ESO-1 and/or MAGEA10). In some aspects, a polynucleotide described herein comprises a single ORF with multiple coding regions, wherein the multiple coding regions are linked and encode multiple KRAS antigens. In some aspects, the multiple KRAS antigens comprise one or more of the following amino acid substitutions: G12A, G12C, G12D, G12R, G12S G12V, and G13D. As described herein, in some aspects, the multiple coding regions are arranged in a particular order within a polynucleotide described herein. Accordingly, in some aspects, a polynucleotide described herein comprises the following coding regions which are linked and arranged in the following order (from 5′ to 3′): (1) first coding region encoding KRAS G12D mutant (i.e., having the G12D amino acid substitution), (2) second coding region encoding KRAS G12V mutant, (3) third coding region encoding KRAS G12C mutant, (4) fourth coding region encoding KRAS G13D mutant, (5) fifth coding region encoding KRAS G12A mutant, (6) sixth coding region encoding KRAS G12R mutant, and (7) seventh coding region encoding KRAS G12S mutant. In some aspects, such a polynucleotide comprises additional linked coding regions (e.g., encoding a co-stimulatory molecule and/or cytokine). For instance, in some aspects, a polynucleotide of the present disclosure comprises multiple coding regions which encode: (1) multiple KRAS mutant antigens, (2) a co-stimulatory molecule (e.g., CD86), and (3) one or more cytokines (e.g., membrane-bound IL-2 and membrane-bound IL-12), wherein multiple KRAS mutant antigens comprising the following mutations are arranged (from 5′ to 3′): G12D, G12V, G12C, G13D, G12A, G12R, and G12S. In some aspects, a polynucleotide described herein comprises the following coding regions which are linked and arranged in the following order (from 5′ to 3′): (1) first coding region encoding KRAS G12S mutant, (2) second coding region encoding KRAS G12R mutant, (3) third coding region encoding KRAS G12A mutant, (4) fourth coding region encoding KRAS G13D mutant, (5) fifth coding region encoding KRAS G12C mutant, (6) sixth coding region encoding KRAS G12V mutant, and (7) seventh coding region encoding KRAS G12D mutant. In some aspects, such a polynucleotide comprises additional linked coding regions (e.g., encoding a co-stimulatory molecule and/or cytokine). For instance, in some aspects, a polynucleotide of the present disclosure comprises multiple coding regions which encode: (1) multiple KRAS mutant antigens, (2) a co-stimulatory molecule (e.g., CD86), and (3) one or more cytokines (e.g., membrane-bound IL-2 and membrane-bound IL-12), wherein multiple KRAS mutant antigens comprising the following mutations are arranged (from 5′ to 3′): G12S, G12R, G21A, G13D, G12C, G12V, and G12D. As demonstrated herein (see, e.g., Examples 6-8), when cells (e.g., PBMCs) are squeezed using such polynucleotides, each of the encoded antigens can be expressed in the PBMCs such that the PBMCs are capable of inducing the activation of T cells that recognize the encoded antigens.

In some aspects, an antigen comprises a self antigen. For instance, in some aspects, an antigen comprises a self-antigen that are overexpressed in certain tumors. In some aspects, an antigen comprises a non-self antigen. As used herein, the term “non-self antigen” refers to any antigenic substance (i.e., capable of inducing an immune response when present in vivo) derived from a pathogen, including but not limited to a virus, fungus, protozoa, or combinations thereof. Non-limiting example of non-self antigens include those derived from one or more of the following: a Human Gamma herpes virus 4 (i.e., Epstein Barr virus (EBV)), influenza A virus, influenza B virus, cytomegalovirus, Staphylococcus aureus, Mycobacterium tuberculosis, Chlamydia trachomatis, HIV (e.g., HIV-2), corona viruses (e.g., COVID-19, MERS-COV, and SARS COV), filoviruses (e.g., Marburg and Ebola), Streptococcus pyogenes, Streptococcus pneumoniae, Plasmodia species (e.g., vivax and falciparum), Chikungunya virus, Human Papilloma virus (HPV), Hepatitis B virus (HBV), Hepatitis C virus (HCV), human T-lymphotropic virus (HTLV1), human herpes virus 8 (HHV8), Merkel cell polyomavirus (MCV), bunyavirus (e.g., hanta virus), arena virus (e.g., LCMV and Lassa virus), flavivirus (e.g., dengue, Zika, Japanese encephalitis, west nile, and yellow fever), enterovirus (e.g., polio), astrovirus (e.g., gastroenteritis), rhabdoviridae (e.g., rabies), Borrelia burgdorferi and Borrelia mayonii (e.g., Lyme disease), herpes simplex virus 2 (HSV-2), Klebsiella sp, Pseudomonas aeruginosa, Enterococcus sp., Proteus sp., Enterobacter sp., Actinobacter sp., coagulase-negative staphylococci (CoNS), Mycoplasma sp., Adenovirus, Adeno-associated virus (AAV), or combinations thereof.

Accordingly, in some aspects, one or more of the coding regions of a polynucleotide described herein encodes for a non-self antigen. For instance, in some aspects, a polynucleotide described herein comprises multiple coding regions, wherein the multiple coding regions are linked, and wherein one or more of the multiple coding regions encode for a non-self antigen. In some aspects, a polynucleotide described herein comprises multiple coding regions, wherein the multiple coding regions are linked, and wherein one or more of the multiple coding regions encode for a HPV antigen (e.g., E7 and/or E6 protein). In some aspects, a polynucleotide described herein comprises multiple coding regions, wherein the multiple coding regions are linked, and wherein one or more of the multiple coding regions encode for a HIV antigen. In some aspects, a polynucleotide described herein comprises multiple coding regions, wherein the multiple coding regions are linked, and wherein one or more of the multiple coding regions encode for a HBV antigen. In some aspects, a polynucleotide described herein comprises multiple coding regions, wherein the multiple coding regions are linked, and wherein one or more of the multiple coding regions encode for a HSV antigen (e.g., HSV gD). In some aspects, a polynucleotide described herein comprises multiple coding regions, wherein the multiple coding regions are linked, and wherein one or more of the multiple coding regions encode for an influenza antigen (e.g., M1 protein). In some aspects, a polynucleotide described herein comprises multiple coding regions, wherein the multiple coding regions are linked, and wherein one or more of the multiple coding regions encode for a coronavirus antigen (e.g., S protein). In some aspects, a polynucleotide described herein comprises multiple coding regions, wherein the multiple coding regions are linked, and wherein the multiple coding regions encode for multiple non-self antigens (e.g., HPV antigen, EBV antigen, HSV antigen, influenza antigen, coronavirus antigen, and combinations thereof). In some aspects, a polynucleotide described herein comprises multiple coding regions, wherein the multiple coding regions are linked, and wherein one or more of the multiple coding regions encode for at least one cancer antigen (e.g., KRAS mutant, NY-ESO-1, MAGE A10, or combinations thereof) and one or more of the multiple coding regions encoded for at least one non-self antigen (e.g., HPV antigen, EBV antigen, HSV antigen, influenza antigen, coronavirus antigen, and combinations thereof).

### Linkers

As described herein, polynucleotides described herein comprise multiple coding regions, wherein the multiple coding regions are linked. Any useful means of linking two moieties (e.g., first coding region and a second coding region) can be used with the present disclosure. In some aspects, the multiple coding regions are linked with a linker. In some aspects, the multiple coding regions can be linked without a linker. For instance, in some aspects, a first coding region and a second coding region can be arranged within an ORF such that they are immediately adjacent to each other, such that when translated, the protein encoded by the first coding region and the protein encoded by the second coding region would be linked by traditional peptide amide-bond. As used herein, the term “linker” refers to a peptide or polypeptide sequence (e.g., a synthetic peptide or polypeptide sequence) or to a non-polypeptide, e.g., an alkyl chain. In some aspects, two or more linkers can be linked in tandem. When multiple linkers are present, each of the linkers can be the same or different. Generally, linkers provide flexibility or prevent/ameliorate steric hindrances. Linkers are not typically cleaved; however in certain aspects, such cleavage can be desirable. Accordingly, in some aspects, a linker can comprise one or more protease-cleavable sites, which can be located within the sequence of the linker or flanking the linker at either end of the linker sequence. Accordingly, in some aspects, a linker useful for the present disclosure comprises a cleavable linker.

As used herein, the term “cleavable linker” refers to a linker that comprises a cleavage site, such that when expressed can be selectively cleaved to produce two or more products. In some aspects, the linker is selected from a P2A linker, a T2A linker, an F2A linker, an E2A linker, a furin cleavage site, or any combination thereof (see Table 1 below). In some aspects, the linker further comprises a GSG linker sequence. In some aspects, a linker useful for the present disclosure comprises an Internal Ribosome Entry Site (IRES), such that separate polypeptides encoded by the first and second genes are produced during translation. Additional description of linkers that can be used with the present disclosure are provided, e.g., in WO 2020/223625 A1 and US 2019/0276801 A1, each of which is incorporated herein by reference in its entirety.

TABLE 1
Linker Sequences

| | |
|---|---|
| P2A | ATNFSLLKQAGDVEENPGP (SEQ ID NO: 18) |
| T2A | EGRGSLLTCGDVEENPGP (SEQ ID NO: 19) |
| F2A | VKQTLNFDLLKLAGDVESNPGP (SEQ ID NO: 20) |
| E2A | QCTNYALLKLAGDVESNPGP (SEQ ID NO: 21) |
| Furin | RAKR (SEQ ID NO: 22) |
| Cleavage | |
| Site | |

In some aspects, the linker is a peptide linker. In some aspects, the peptide linker comprises a glycine/serine linker. In some aspects, the peptide linker is glycine/serine linker according to the formula [(Gly)n-Ser]m (SEQ ID NO: 23), where n is any integer from 1 to 100 and m is any integer from 1 to 100. In some aspects, the glycine/serine linker is according to the formula [(Gly)x-(Ser)y]z (SEQ ID NO: 24), wherein x in an integer from 1 to 4, y is 0 or 1, and z is an integers from 1 to 50. In some aspects, the peptide linker comprises the sequence Gn (SEQ ID NO: 25), where n can be an integer from 1 to 100. In some aspects, the peptide linker can comprise the sequence (GlyAla)n (SEQ ID NO: 26), wherein n is an integer between 1 and 100. In some aspects, the peptide linker can comprise the sequence (GlyGlySer)n (SEQ ID NO: 27), wherein n is an integer between 1 and 100. In some aspects, the peptide linker comprises the sequence GGGG (SEQ ID NO: 28).

In some aspects, the peptide linker comprises the sequence (GGGS)n (SEQ ID NO: 29). In certain aspects, the peptide linker comprises the sequence (GGS)n (GGGGS)n (SEQ ID NO: 30). In such aspects, n can be an integer from 1 to 100. In some aspects, n can be an integer from one to 20, i.e., 1, 2, 3, 4, 5, 6, 7, 8, 9, 10, 11, 12, 13, 14, 15, 16, 17, 18, 19, or 20. In some aspects, n is an integer from 1 to 100. In some aspects, the peptide linker is (G4S) 3 (SEQ ID NO: 31).

In some aspects, the peptide linker comprises a EAAAK linker. In some aspects, the EAAAK linker comprises the sequence (EAAAK) n, wherein n is an integer between 1 to 10 (i.e., 1, 2, 3, 4, 5, 6, 7, 8, 9, or 10). In some aspects, the peptide linker is (EAAAK) 3 (SEQ ID NO: 32).

In some aspects, the peptide linker is synthetic, i.e., non-naturally occurring. In one aspect, a peptide linker includes peptides (or polypeptides) (e.g., natural or non-naturally occurring peptides) which comprise an amino acid sequence that links or genetically fuses a first linear sequence of amino acids to a second linear sequence of amino acids to which it is not naturally linked or genetically fused in nature. For example, in one aspect, the peptide linker can comprise non-naturally occurring polypeptides which are modified forms of naturally occurring polypeptides (e.g., comprising a mutation such as an addition, substitution or deletion).

In some aspects, the peptide linker can comprise non-naturally occurring amino acids. In yet other aspects, the peptide linker can comprise naturally occurring amino acids occurring in a linear sequence that does not occur in nature. In still other aspects, the peptide linker can comprise a naturally occurring polypeptide sequence.

In some aspects, the linker comprises a non-peptide linker. In other aspects, the linker consists of a non-peptide linker. In some aspects, the non-peptide linker can be, e.g., maleimido caproyl (MC), maleimido propanoyl (MP), methoxyl polyethyleneglycol (MPEG), succinimidyl 4-(N-maleimidomethyl)-cyclohexane-1-carboxylate (SMCC), m-maleimidobenzoyl-N-hydroxysuccinimide ester (MBS), succinimidyl 4-(p-maleimidophenyl) butyrate (SMPB), N-succinimidyl (4-iodoacetyl)aminobenzonate (SIAB), succinimidyl 6-[3-(2-pyridyldithio)-propionamide]hexanoate (LC-SPDP), 4-succinimidyloxycarbonyl-alpha-methyl-alpha-(2-pyridyldithio) toluene (SMPT), etc. (see, e.g., U.S. Pat. No. 7,375,078, which is herein incorporated by reference in its entirety).

### Other Components:

In addition to any of the features described above, a polynucleotide useful for the present disclosure can comprise one or more additional components. In some aspects, these additional components can aid in the polynucleotide exerting its therapeutic effect (e.g., allow for improved translation and/or increase stability of the polynucleotide). Non-limiting examples of additional components that can be included in the polynucleotides described herein include: an Internal Ribosome Entry Site (IRES), an intron sequence, a homology arma promoter, an enhancer, a UTR, a sequence encoding a signal peptide, a translation initiation sequence, a 3′ tailing region of linked nucleosides, a 5′ cap, a sequence encoding a 2A ribosome skip peptide, or any combination thereof.

### Untranslated Regions (UTRs)

In some aspects, a polynucleotide described herein comprises one or more UTR sequences (e.g., 5′-UTR and/or 3′-UTR). As used herein, the term “untranslated region” or “UTR” refers to a region of a gene that is transcribed but not translated. The “5′-UTR” starts at the transcription start site and continues to the start codon but does not include the start codon; whereas, the “3′-UTR” starts immediately following the stop codon and continues until the transcriptional termination signal. There is growing body of evidence about the regulatory roles played by the UTRs in terms of stability of the nucleic acid molecule and translation. The regulatory features of a UTR can be incorporated into a polynucleotide described herein to enhance the stability of the molecule. The specific features can also be incorporated to ensure controlled down-regulation of the transcript in case they are misdirected to undesired organs sites.

In some aspects, UTRs useful for the present disclosure comprise those that are present in genes that are abundantly expressed in specific cells, tissues, and/or organs. By including such additional UTRs, in some aspects, a polynucleotide of the present disclosure can be preferentially expressed in specific cells, tissues, and/or organs, e.g., when administered to a subject. For example, additionally introducing a UTR (e.g., 5-UTR) of a mRNA expressed in the liver (e.g., albumin, serum amyloid A, Apolipoprotein A/B/E, transferrin, alpha fetoprotein, erythropoietin, or Factor VIII) can enhance the expression of a polynucleotide described herein in hepatic and/or liver cell lines. Non-limiting examples of such tissue-specific UTRs include those from: (a) muscle: myoD, myosin, myoglobin, myogenin, and herculin; (b) endothelial cells: Tie-1 and CD36; (c) myeloid cells: C/EBP, AML1, G-CSF, GM-CSF, CD11b, MSR, Fr-1, and i-NOS; (d) leukocytes: CD45 and CD18; (e) adipose tissue: CD36, GLUT4, ACRP30, and adiponectin; and (f) lung epithelial cells: SP-A/B/C/D.

Additional examples of UTRs that can be used with the present disclosure include one or more 5′-UTR and/or 3′-UTR derived from the nucleic acid sequence of: a globin, such as an α- or β-globin (e.g., a Xenopus, mouse, rabbit, or human globin); a strong Kozak translational initiation signal; a CYBA (e.g., human cytochrome b-245 α polypeptide); an albumin (e.g., human albumin7); a HSD17B4 (hydroxysteroid (17-β) dehydrogenase); a virus (e.g., a tobacco etch virus (TEV), a Venezuelan equine encephalitis virus (VEEV), a Dengue virus, a cytomegalovirus (CMV) (e.g., CMV immediate early 1 (IE1)), a hepatitis virus (e.g., hepatitis B virus), a sindbis virus, or a PAV barley yellow dwarf virus); a heat shock protein (e.g., hsp70); a translation initiation factor (e.g., elF4G); a glucose transporter (e.g., hGLUT1 (human glucose transporter 1)); an actin (e.g., human a or β actin); a GAPDH; a tubulin; a histone; a citric acid cycle enzyme; a topoisomerase (e.g, a 5′-UTR of a TOP gene lacking the 5′ TOP motif (the oligopyrimidine tract)); a ribosomal protein Large 32 (L32); a ribosomal protein (e.g., human or mouse ribosomal protein, such as, for example, rps9); an ATP synthase (e.g., ATP5A1 or the β subunit of mitochondrial H⁺-ATP synthase); a growth hormone e (e.g., bovine (bGH) or human (hGH)); an elongation factor (e.g., elongation factor 1 α1 (EEF1A1)); a manganese superoxide dismutase (MnSOD); a myocyte enhancer factor 2A (MEF2A); a β-F1-ATPase, a creatine kinase, a myoglobin, a granulocyte-colony stimulating factor (G-CSF); a collagen (e.g., collagen type I, alpha 2 (Col1A2), collagen type I, alpha 1 (Col1A1), collagen type VI, alpha 2 (Col6A2), collagen type VI, alpha 1 (Col6A1)); a ribophorin (e.g., ribophorin I (RPNI)); a low density lipoprotein receptor-related protein (e.g., LRP1); a cardiotrophin-like cytokine factor (e.g., Nnt1); calreticulin (Calr); a procollagen-lysine, 2-oxoglutarate 5-dioxygenase 1 (Plod1); a nucleobindin (e.g., Nucb1); and combinations thereof.

### Cap Structure

In some aspects, a polynucleotide described herein (e.g., comprising multiple coding regions that are linked) comprises a 5′-cap.

As used herein, the term “5′-cap” refers to a modified nucleotide (e.g., guanine) that can be added to the 5′-end of a polynucleotide (e.g., mRNA). The 5′-cap structure can play a role in the nuclear export of the polynucleotide (e.g., to the cytoplasm where translation can occur) and/or promote the stability of the polynucleotide. In some aspects, the 5′-cap can be linked to the 5′-terminal end of the polynucleotide described herein via a 5′-5′-triphosphate linkage. In certain aspects, the 5′-cap can be methylated (e.g., m⁷GpppN, wherein N is the terminal 5′ nucleotide of the polynucleotide). Any suitable 5′-cap known in the art can be used with the present disclosure. Non-limiting examples of 5′-caps that can be used with the present disclosure include: m₂^{7,2′—O}GppₛpGRNA, m⁷GpppG, m⁷Gppppm⁷G, m₂^{(7,3′—O)}GpppG, m₂^{(7,2′O)}GppspG (D1), m₂^{(7,2′—O)}GppspG (D2), m₂^{7,3-O}Gppp(m₁^{2′-O})ApG, (m⁷G-3′ mppp-G; which can equivalently be designated 3′O-Me-m7G (5′)ppp(5′)G), N7,2′-O-dimethyl-guanosine-5′-triphosphate-5′-guanosine, m⁷Gm-ppp-G, N7-(4-chlorophenoxyethyl)-G(5′)ppp(5′)G, N7-(4-chlorophenoxyethyl)-m^{3′-O}G(5′)ppp(5′)G, 7 mG(5′)ppp(5′)NlpN2p, 7 mG(5′)ppp(5′)NlmpNp. 7 mG(5′)-ppp(5′) NlmpN2 mp, m (7)Gpppm(3) (6,6,2′)Apm (2′)Apm (2′)Cpm(2) (3,2′)Up, inosine, N1-methyl-guanosine, 2′ fluoro-guanosine, 7-deaza-guanosine, 8-oxo-guanosine, 2-amino-guanosine, LNA-guanosine, 2-azido-guanosine, N1-methylpseudouridine, m7G (5′)ppp(5′)(2′OMeA)pG, or combinations thereof.

In some aspects, a 5′-cap that can be used with a polynucleotide of the present disclosure comprises a cap analog (also knowns as a “synthetic cap analog.” “chemical cap,” “chemical cap analog,” or “structural or functional cap analog”). “Cap analog” differs from natural (i.e., endogenous, wild-type, or physiological) 5′-caps in their chemical structure, while retaining cap function. Non-limiting examples of cap analogs are described in U.S. Pat. No. 8,519,110 and Kore et al., Bioorganic & Medicinal Chemistry 21:4570-4574 (2013), each of which is incorporated herein by reference in its entirety.

In some aspects, a 5′-cap is modified. A modification on the 5′-cap can further increase the stability, half-life, and/or translational efficiency of the polynucleotide. In some aspects, a modified 5′ cap comprises one or more of the following modifications: modification at the 2′ and/or 3′ position of a capped guanosine triphosphate (GTP), a replacement of the sugar ring oxygen (that produced the carbocyclic ring) with a methylene moiety (CH2), a modification at the triphosphate bridge moiety of the cap structure, or a modification at the nucleobase (G) moiety. See, e.g., US 2014/0147454 and WO 2018/160540, each of which is incorporated herein by reference in its entirety.

### Poly (A) Tail

In some aspects, a polynucleotide described herein (e.g., comprising multiple coding regions that are linked) comprises a long chain of adenine nucleotides (referred to herein as “poly(A) tail”) at the 3′-end of the polynucleotide. In some aspects, the poly(A) tail is present alone or in combination with other components described herein (e.g., 5′-cap).

In some aspects, the length of the poly(A) tail is greater than about 30 nucleotides in length. In certain aspects, the length of the poly(A) tail is at least about 35 nucleotides, at least about 40 nucleotides, at least about 45 nucleotides, at least about 50 nucleotides, at least about 60 nucleotides, at least about 70 nucleotides, at least about 80 nucleotides, at least about 90 nucleotides, at least about 100 nucleotides, at least about 110 nucleotides, at least about 120 nucleotides, at least about 130 nucleotides, at least about 140 nucleotides, at least about 150 nucleotides, at least about 160 nucleotides, at least about 170 nucleotides, at least about 180 nucleotides, at least about 190 nucleotides, at least about 200 nucleotides, at least about 250 nucleotides, at least about 300 nucleotides, at least about 350 nucleotides, at least about 400 nucleotides, at least about 450 nucleotides, or at least about 500 nucleotides or more.

Any suitable poly(A) tails known in the art can be used with the present disclosure. Non-limiting examples of poly(A) tails that can be used with the present disclosure include: SV40 poly(A), bGH poly(A), actin poly(A), hemoglobin poly(A), poly(A)-G quartet, or combinations thereof.

### Enhancers

In some aspects, the expression of an antigen encoded by the one or more coding regions of a polynucleotide described herein can be further increased using one or more enhancer sequences (also referred to herein as “translation enhancer element” or “TEE”). Accordingly, in some aspects, a polynucleotide described herein (e.g., comprising multiple coding regions that are linked) comprises one or more enhancer sequences. In some aspects, a polynucleotide described herein comprises at least about 1, at least about 2, at least about 3, at least about 4, at least about 5, at least about 6, at least about 7, at least about 8, at least about 9, at least about 10, at least about 15, at least about 20, at least about 25, at least about 30, at least about 35, at least about 40, at least about 45, or at least about 50 or more enhancer sequences. Where a polynucleotide comprises multiple enhancers, in some aspects, each of the enhancers is the same. In some aspects, one or more of the enhancers different. In some aspects, one or more of the enhancers are separated by a spacer.

Any enhancers known in the art can be used with the polynucleotides of the present disclosure. See, e.g., WO1999024595, WO2012009644, WO2009075886 and WO2007025008, European Patent Publication No. EP2610341A1 and EP2610340A1, U.S. Pat. Nos. 6,310,197, 6,849,405, 7,456,273, 7,183,395, each of which is herein incorporated by reference in its entirety. In some aspects, an enhancer useful for the present disclosure is a tissue-specific enhancer. In certain aspects, an enhancer that can be used with the present disclosure is selected from a human skeletal actin gene element, a cardiac actin gene element, a myocyte-specific enhancer binding factor MEF (e.g. MEF2), a MyoD enhancer element, a cardiac enhancer factor (CEF) site, murine creatine kinase enhancer element, skeletal fast-twitch troponin C gene element, slow-twitch cardiac troponin C gene element, the slow-twitch troponin I gene element, hypozia-inducible nuclear factors, steroid-inducible element, glucocorticoid response element (GRE), or any combination thereof.

### IRES Sequence

In some aspects, a polynucleotide described herein (e.g., comprising multiple coding regions that are linked) can further comprise a nucleotide sequence encoding an internal ribosome-entry site (IRES). IRES plays an important role in initiating protein synthesis in absence of the 5′-cap structure. An IRES can act as the sole ribosome binding site, or can serve as one of multiple ribosome binding sites of a polynucleotide. Polynucleotides containing more than one functional ribosome binding site can encode several peptides or polypeptides that are translated independently by the ribosomes (“polycistronic polynucleotide”). Accordingly, where a polynucleotide described herein comprises a sequence encoding an IRES, in certain aspects, the polynucleotide can comprise multiple (e.g., at least two) translatable regions—e.g., a nucleotide sequence encoding a coronavirus spike protein; and a nucleotide sequence encoding a different coronavirus protein (e.g., nucleocapsid protein).

Any IRES sequences known in the art can be used with the present disclosure. Non-limiting examples of IRES sequences that can be used with the present disclosure include those from picornaviruses (e.g. FMDV), pest viruses (CFFV), polio viruses (PV), encephalomyocarditis viruses (ECMV), foot-and-mouth disease viruses (FMDV), hepatitis C viruses (HCV), classical swine fever viruses (CSFV), murine leukemia virus (MLV), simian immune deficiency viruses (SIV), cricket paralysis viruses (CrPV), or combinations thereof.

### Post-Transcriptional Regulatory Elements

In some aspects, an additional component that can be used with a polynucleotide described herein (e.g., comprising multiple coding regions that are linked) comprises a post-transcriptional regulatory element. In some aspects, the post-transcription regulatory element can be present in a polynucleotide described herein in combination with one or more other components described herein (e.g., 5′-cap, 3′-poly(A) tail, enhancer sequences, IRES sequence, or combinations thereof). In some aspects, the post-translational regulatory element is positioned 3′- to the multiple coding regions of a polynucleotide described herein. Non-limiting examples of post-transcriptional regulatory elements that are useful for the present disclosure include a mutated woodchuck hepatitis virus post-transcriptional regulatory element (WPRE), microRNA binding site, DNA nuclear targeting sequence, or combinations thereof.

In some aspects, the one or more additional components that can be present in a polynucleotide described herein (e.g., comprising multiple coding regions that are linked) comprises a promoter. In certain aspects, a polynucleotide can include a single promoter. In some aspects, a polynucleotide can include multiple promoters (e.g., two, three, four, or five or more) that are operably linked to the multiple coding regions of a polynucleotide described herein. Where a polynucleotide comprises multiple promoters, in some aspects, each of the multiple promoters are the same. In certain aspects, one or more of the multiple promoters are different.

In some aspects, a promoter useful for the present disclosure comprises a mammalian promoter, viral promoter, or both. In certain aspects, a promoter that can be used with the polynucleotides described herein comprises a constitutive promoter, an inducible promoter, or both.

Constitutive mammalian promoters include, but are not limited to, the promoters for the following genes: hypoxanthine phosphoribosyl transferase (HPRT), adenosine deaminase, pyruvate kinase, beta-actin promoter, and other constitutive promoters. Exemplary viral promoters which function constitutively in eukaryotic cells include, for example, promoters from the cytomegalovirus (CMV), simian virus (e.g., SV40), papilloma virus, adenovirus, human immunodeficiency virus (HIV), Rous sarcoma virus, cytomegalovirus, the long terminal repeats (LTR) of Moloney leukemia virus, and other retroviruses, and the thymidine kinase promoter of herpes simplex virus. As described herein, in some aspects, a promoter that can be used with the present disclosure is an inducible promoter. Inducible promoters are expressed in the presence of an inducing agent. For example, the metallothionein promoter is induced to promote transcription and translation in the presence of certain metal ions. In some aspects, a promoter that can be used comprises the T7 promoter.

### Modified Nucleosides/Nucleotides

In some aspects, a polynucleotide described herein (e.g., comprising multiple coding regions that are linked) comprises at least one chemically modified nucleoside and/or nucleotide.

A “nucleoside” refers to a compound containing a sugar molecule (e.g., a pentose or ribose) or a derivative thereof in combination with an organic base (e.g., a purine or pyrimidine) or a derivative thereof (also referred to herein as “nucleobase”). A “nucleotide” refers to a nucleoside including a phosphate group. Modified nucleotides can be synthesized by any useful method, such as, for example, chemically, enzymatically, or recombinantly, to include one or more modified or non-natural nucleosides.

Polynucleotides can comprise a region or regions of linked nucleosides. Such regions can have variable backbone linkages. The linkages can be standard phosphodiester linkages, in which case the polynucleotides would comprise regions of nucleotides.

A polynucleotide of the present disclosure can comprise various distinct modifications. In some aspects, a polynucleotide can contain one, two, or more (optionally different) nucleoside or nucleotide modifications. In some aspects, a polynucleotide can exhibit one or more desirable properties, e.g., improved thermal or chemical stability, reduced immunogenicity, reduced degradation, increased binding to the target microRNA, reduced non-specific binding to other microRNA or other molecules, as compared to an unmodified polynucleotide.

In some aspects, a polynucleotide of the present disclosure is chemically modified. As used herein, in reference to a polynucleotide, the terms “chemical modification” or, as appropriate, “chemically modified” refer to modification with respect to adenosine (A), guanosine (G), uridine (U), thymidine (T) or cytidine (C) ribo- or deoxyribonucleosides in one or more of their position, pattern, percent or population, including, but not limited to, its nucleobase, sugar, backbone, or any combination thereof.

In some aspects, a polynucleotide of the present disclosure can have a uniform chemical modification of all or any of the same nucleoside type or a population of modifications produced by downward titration of the same starting modification in all or any of the same nucleoside type, or a measured percent of a chemical modification of all any of the same nucleoside type but with random incorporation. In further aspects, the polynucleotide of the present disclosure can have a uniform chemical modification of two, three, or four of the same nucleoside type throughout the entire polynucleotide (such as all uridines and/or all cytidines, etc. are modified in the same way).

Modified nucleotide base pairing encompasses not only the standard adenine-thymine, adenine-uracil, or guanine-cytosine base pairs, but also base pairs formed between nucleotides and/or modified nucleotides comprising non-standard or modified bases, wherein the arrangement of hydrogen bond donors and hydrogen bond acceptors permits hydrogen bonding between a non-standard base and a standard base or between two complementary non-standard base structures. One example of such non-standard base pairing is the base pairing between the modified nucleobase inosine and adenine, cytosine or uracil. Any combination of base/sugar or linker can be incorporated into polynucleotides of the present disclosure.

The skilled artisan will appreciate that, except where otherwise noted, polynucleotide sequences set forth in the instant application will recite “T”s in a representative DNA sequence but where the sequence represents RNA, the “T”s would be substituted for “U”s. For example, TD's of the present disclosure can be administered as RNAs, as DNAs, or as hybrid molecules comprising both RNA and DNA units.

In some aspects, the polynucleotide described herein includes a combination of at least two (e.g., 2, 3, 4, 5, 6, 7, 8, 8, 10, 11, 12, 13, 14, 15, 16, 17, 18, 18, 20 or more) modified nucleobases.

In some aspects, the nucleobases, sugar, backbone linkages, or any combination thereof in a polynucleotide are modified by at least about 5%, at least 10%, at least 15%, at least 20%, at least 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99% or 100%.

### Base Modifications

In some aspects, the chemical modification is at nucleobases in a polynucleotide of the present disclosure (e.g., comprising multiple coding regions that are linked). In some aspects, the at least one chemically modified nucleoside is a modified uridine (e.g., pseudouridine (v), 2-thiouridine (s2U), 1-methyl-pseudouridine (m1ψ), 1-ethyl-pseudouridine (e1ψ), or 5-methoxy-uridine (mo5U)), a modified cytosine (e.g., 5-methyl-cytidine (m5C)) a modified adenosine (e.g, 1-methyl-adenosine (m1A), N6-methyl-adenosine (m6A), or 2-methyl-adenine (m2A)), a modified guanosine (e.g., 7-methyl-guanosine (m7G) or 1-methyl-guanosine (m1G)), or a combination thereof.

In some aspects, the polynucleotide described herein is uniformly modified (e.g., fully modified, modified throughout the entire sequence) for a particular modification. For example, a polynucleotide can be uniformly modified with the same type of base modification, e.g., 5-methyl-cytidine (m5C), meaning that all cytosine residues in the polynucleotide sequence are replaced with 5-methyl-cytidine (m5C). Similarly, a polynucleotide can be uniformly modified for any type of nucleoside residue present in the sequence by replacement with a modified nucleoside such as any of those set forth above.

In some aspects, the polynucleotide includes a combination of at least two (e.g., 2, 3, 4 or more) of modified nucleobases. In some aspects, at least about 5%, at least 10%, at least 15%, at least 20%, at least 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99% or 100% of a type of nucleobases in a polynucleotide of the present disclosure are modified nucleobases.

### Backbone Modification

In some aspects, a polynucleotide described herein can include any useful linkage between the nucleosides. Such linkages, including backbone modifications, that are useful in the composition of the present disclosure include, but are not limited to the following: 3′-alkylene phosphonates, 3′-amino phosphoramidate, alkene containing backbones, aminoalkylphosphoramidates, aminoalkylphosphotriesters, boranophosphates, —CH₂—O—N(CH₃)—CH₂—, —CH₂—N(CH₃)—N(CH₃)—CH₂—, —CH₂—NH—CH₂—, chiral phosphonates, chiral phosphorothioates, formacetyl and thioformacetyl backbones, methylene (methylimino), methylene formacetyl and thioformacetyl backbones, methyleneimino and methylenehydrazino backbones, morpholino linkages, —N(CH₃)—CH₂—CH₂—, oligonucleosides with heteroatom internucleoside linkage, phosphinates, phosphoramidates, phosphorodithioates, phosphorothioate internucleoside linkages, phosphorothioates, phosphotriesters, PNA, siloxane backbones, sulfamate backbones, sulfide sulfoxide and sulfone backbones, sulfonate and sulfonamide backbones, thionoalkylphosphonates, thionoalkylphosphotriesters, and thionophosphoramidates.

In some aspects, the presence of a backbone linkage disclosed above increase the stability and resistance to degradation of a polynucleotide of the present disclosure. In some aspects, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99% or 100% of the backbone linkages in a polynucleotide of the present disclosure are modified (e.g., all of them are phosphorothioate).

In some aspects, a backbone modification that can be included in a polynucleotide of the present disclosure comprises phosphorodiamidate morpholino oligomer (PMO) and/or phosphorothioate (PS) modification.

### Sugar Modification

The modified nucleosides and nucleotides which can be incorporated into a polynucleotide of the present disclosure can be modified on the sugar of the nucleic acid. Incorporating affinity-enhancing nucleotide analogues, such as LNA or 2′-substituted sugars, can allow the length and/or the size of the polynucleotide to be modified (e.g., reduced).

In some aspects, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99% or 100% of the nucleotides in a polynucleotide of the present disclosure contain sugar modifications (e.g., LNA).

As described herein, in some aspects, a polynucleotide described herein can be a RNA (e.g., mRNA). Generally, RNA includes the sugar group ribose, which is a 5-membered ring having an oxygen. Exemplary, non-limiting modified nucleotides include replacement of the oxygen in ribose (e.g., with S, Se, or alkylene, such as methylene or ethylene); addition of a double bond (e.g., to replace ribose with cyclopentenyl or cyclohexenyl); ring contraction of ribose (e.g., to form a 4-membered ring of cyclobutane or oxetane); ring expansion of ribose (e.g., to form a 6- or 7-membered ring having an additional carbon or heteroatom, such as for anhydrohexitol, altritol, mannitol, cyclohexanyl, cyclohexenyl, and morpholino that also has a phosphoramidate backbone); multicyclic forms (e.g., tricyclo; and “unlocked” forms, such as glycol nucleic acid (GNA) (e.g., R-GNA or S-GNA, where ribose is replaced by glycol units attached to phosphodiester bonds), threose nucleic acid (TNA, where ribose is replace with α-L-threofuranosyl-(3′→2′)), and peptide nucleic acid (PNA, where 2-amino-ethyl-glycine linkages replace the ribose and phosphodiester backbone). The sugar group can also contain one or more carbons that possess the opposite stereochemical configuration than that of the corresponding carbon in ribose. Thus, a polynucleotide molecule can include nucleotides containing, e.g., arabinose, as the sugar.

The 2′ hydroxyl group (OH) of ribose can be modified or replaced with a number of different substituents. Exemplary substitutions at the 2′-position include, but are not limited to, H, halo, optionally substituted C₁₋₆ alkyl; optionally substituted C₁₋₆ alkoxy; optionally substituted C₆₋₁₀ aryloxy; optionally substituted C₃₋₈ cycloalkyl; optionally substituted C₃₋₈ cycloalkoxy; optionally substituted C₆₋₁₀ aryloxy; optionally substituted C₆₋₁₀ aryl-C₁₋₆ alkoxy, optionally substituted C₁₋₁₂ (heterocyclyl)oxy; a sugar (e.g., ribose, pentose, or any described herein); a polyethyleneglycol (PEG), —O(CH₂CH₂O)ₙCH₂CH₂OR, where R is H or optionally substituted alkyl, and n is an integer from 0 to 20 (e.g., from 0 to 4, from 0 to 8, from 0 to 10, from 0 to 16, from 1 to 4, from 1 to 8, from 1 to 10, from 1 to 16, from 1 to 20, from 2 to 4, from 2 to 8, from 2 to 10, from 2 to 16, from 2 to 20, from 4 to 8, from 4 to 10, from 4 to 16, and from 4 to 20); “locked” nucleic acids (LNA) in which the 2′-hydroxyl is connected by a C₁₋₆ alkylene or C₁₋₆ heteroalkylene bridge to the 4′-carbon of the same ribose sugar, where exemplary bridges include methylene, propylene, ether, amino bridges, aminoalkyl, aminoalkoxy, amino, and amino acid.

In some aspects, nucleotide analogues present in a polynucleotide of the present disclosure comprise, e.g., 2′-O-alkyl-RNA units, 2′-OMe-RNA units, 2′-O-alkyl-SNA, 2′-amino-DNA units, 2′-fluoro-DNA units, LNA units, arabino nucleic acid (ANA) units, 2′-fluoro-ANA units, HNA units, INA (intercalating nucleic acid) units, 2′MOE units, or any combination thereof. In some aspects, the LNA is, e.g., oxy-LNA (such as beta-D-oxy-LNA, or alpha-L-oxy-LNA), amino-LNA (such as beta-D-amino-LNA or alpha-L-amino-LNA), thio-LNA (such as beta-D-thio0-LNA or alpha-L-thio-LNA), ENA (such a beta-D-ENA or alpha-L-ENA), or any combination thereof. In further aspects, nucleotide analogues that can be included in a polynucleotide of the present disclosure comprises a locked nucleic acid (LNA), an unlocked nucleic acid (UNA), an arabino nucleic acid (ABA), a bridged nucleic acid (BNA), and/or a peptide nucleic acid (PNA).

In some aspects, a polynucleotide of the present disclosure can comprise both modified RNA nucleotide analogues (e.g., LNA) and DNA units. In some aspects, a polynucleotide described herein is a gapmer. See, e.g., U.S. Pat. Nos. 8,404,649; 8,580,756; 8,163,708; 9,034,837; all of which are herein incorporated by reference in their entireties.

In some aspects, a polynucleotide of the present disclosure can include modifications to prevent rapid degradation by endo- and exo-nucleases. Modifications include, but are not limited to, for example, (a) end modifications, e.g., 5′ end modifications (phosphorylation, dephosphorylation, conjugation, inverted linkages, etc.), 3′ end modifications (conjugation, DNA nucleotides, inverted linkages, etc.), (b) base modifications, e.g., replacement with modified bases, stabilizing bases, destabilizing bases, or bases that base pair with an expanded repertoire of partners, or conjugated bases, (c) sugar modifications (e.g., at the 2′ position or 4′ position) or replacement of the sugar, as well as (d) internucleoside linkage modifications, including modification or replacement of the phosphodiester linkages.

### IV. Vectors

In some aspects, provided herein are vectors (e.g., expression vectors) comprising a polynucleotide described herein (e.g., comprising multiple coding regions that are linked). Suitable vectors for the disclosure include, but are not limited to, expression vectors, viral vectors, and plasmid vectors.

As used herein, an “expression vector” refers to any nucleic acid construct which contains the necessary elements for the transcription and translation of an inserted coding sequence, or in the case of a RNA viral vector, the necessary elements for replication and translation, when introduced into an appropriate host cell. Expression vectors can include plasmids, phagemids, viruses, and derivatives thereof.

As used herein, “viral vectors” include, but are not limited to, nucleic acid sequences from the following viruses: retrovirus, such as Moloney murine leukemia virus, Harvey murine sarcoma virus, murine mammary tumor virus, and Rous sarcoma virus; lentivirus; adenovirus; adeno-associated virus; SV40-type viruses; polyomaviruses; Epstein-Barr viruses; papilloma viruses; herpes virus; vaccinia virus; polio virus; and RNA virus such as a retrovirus. Certain viral vectors are based on non-cytopathic eukaryotic viruses in which non-essential genes have been replaced with the gene of interest. Non-cytopathic viruses include retroviruses, the life cycle of which involves reverse transcription of genomic viral RNA into DNA with subsequent proviral integration into host cellular DNA.

In some aspects, a vector is derived from an adeno-associated virus. In some aspects, a vector is derived from a lentivirus. Examples of the lentiviral vectors are disclosed in WO9931251, WO9712622, WO9817815, WO9817816, and WO9818934, each which is incorporated herein by reference in its entirety.

Other vectors include plasmid vectors. See, e.g., Sambrook et al., Molecular Cloning: A Laboratory Manual, Second Edition, Cold Spring Harbor Laboratory Press, 1989. In the last few years, plasmid vectors have been found to be particularly advantageous for delivering genes to cells in vivo because of their inability to replicate within and integrate into a host genome. These plasmids, however, having a promoter compatible with the host cell, can express a peptide from a gene operably encoded within the plasmid. Some commonly used plasmids available from commercial suppliers include pBR322, pUC18, pUC19, various pcDNA plasmids, pRC/CMV, various pCMV plasmids, pSV40, and pBlueScript. Additional examples of specific plasmids include pcDNA3.1, catalog number V79020; pcDNA3.1/hygro, catalog number V87020; pcDNA4/myc-His, catalog number V86320; and pBudCE4.1, catalog number V53220, all from Invitrogen (Carlsbad, CA). Additionally, plasmids can be custom designed using standard molecular biology techniques to remove and/or add specific fragments of DNA.

### V. Cells

In some aspects, provided herein are cells comprising any of the polynucleotides described herein (e.g., comprising multiple coding regions that are linked). In some aspects, cells described herein have been modified to comprise a polynucleotide described herein, such that the cell expresses the antigens encoded by the multiple coding regions. As demonstrated herein, such antigens are simultaneously expressed in the cells. For instance, in some aspects, cells described herein have been modified with a polynucleotide comprising at least a first coding region and a second coding region, wherein the first coding region and the second coding region are linked. Such modified cells express both the antigen of the first coding region and the antigen of the second region. In some aspects, the cells do not naturally express the multiple antigens, such that the cells express the multiple antigens only after a polynucleotide described herein is introduced into the cell. In some aspects, the cells naturally express one or more of the multiple antigens, but after the cells are modified to comprise a polynucleotide described herein, the expression of one or more of the multiple antigens is increased.

Unless indicated otherwise, any of the polynucleotides described herein can be introduced into a cell using any suitable methods known in the art. Non-limiting examples of suitable methods for delivering one or more exogenous nucleotide sequences to a cell include: transfection (also known as transformation and transduction), electroporation, non-viral delivery, viral transduction, lipid nanoparticle delivery, and combinations thereof. As demonstrated herein, in some aspects, a polynucleotide described herein (e.g., comprising multiple coding regions that are linked) can be introduced into a cell using a constriction-mediated delivery described herein. As further described elsewhere in the present disclosure, as the cells pass through the constriction, they become transiently deformed, such that cell membrane of the cells is perturbed. The perturbation within the cell membrane can allow various payloads (e.g., polynucleotides comprising multiple coding regions that are linked) to enter the cells through the perturbation (e.g., through diffusion). The specific process by which the cells pass through a constriction and become transiently deformed is referred to herein as “squeeze processing,” “squeeze delivery,” or “squeezing.”

Accordingly, in some aspects, the present disclosure provides cells which have been modified to simultaneously express multiple antigens that are not naturally expressed by the cells (e.g., described herein, e.g., cancer antigen and/or non-self antigen), wherein the cells have been passed through a constriction under a set of parameters, thereby causing a perturbation within the cells such that a polynucleotide entered the cells through the perturbation when contacted with the cells, and wherein the polynucleotide comprises multiple coding regions that are linked and encode for the multiple antigens. For instance, in some aspects, a cell provided herein simultaneously expresses at least a first antigen, a second antigen, and a third antigen, wherein the first, second, and third antigens are not naturally expressed by the cells, wherein the cells have been passed through a constriction under a set of parameters, thereby causing a perturbation within the cell such that a polynucleotide entered the cell through the perturbation when contacted with the cell, and wherein the polynucleotide comprises a first coding region encoding the first antigen, a second coding region encoding the second antigen, and a third coding region encoding the third antigen, and wherein the first coding region, the second coding region, and the third coding region are linked.

Any suitable cells known in the art can be modified as described herein.

In some aspects, the cells are stem cells. As used herein, the term “stem cells” refer to cells having not only self-replication ability but also the ability to differentiate into other types of cells (e.g., neurons). In some aspects, stem cells useful for the present disclosure comprise induced pluripotent stem cells (iPSCs), embryonic stem cells (ESCs), tissue-specific stem cells (e.g., liver stem cells, cardiac stem cells, or neural stem cells), mesenchymal stem cells, hematopoietic stem cells (HSCs), or combinations thereof. In some aspects, the stem cells are iPSCs.

In some aspects, the cells are somatic cells. As used herein, the term “somatic cells” refer to any cell in the body that are not gametes (sperm or egg), germ cells (cells that go on to become gametes), or stem cells. Non-limiting examples of somatic cells include blood cells, bone cells, muscle cells, nerve cells, or combinations thereof. In some aspects, somatic cells useful for the present disclosure comprise blood cells. In some aspects, the blood cells are peripheral blood mononuclear cells (PBMCs). As used herein, “PBMCs” refer to any peripheral blood cells having a round nucleus. In some aspects, PBMCs comprise an immune cell. As used herein the term “immune cell” refers to any cell that plays a role in immune function. In some aspects, immune cell comprises a T cell, B cell, natural killer (NK) cell, dendritic cell (DC), NKT cell, mast cell, monocyte, macrophage, basophil, eosinophil, neutrophil, DC2.4 dendritic cell, or combinations thereof. In some aspects, the blood cells are red blood cells. In some aspects, the cell is a cancer cell. In some aspects, the cancer cell is a cancer cell line cell, such as a HeLa cell. In some aspects, the cancer cell is a tumor cell. In some aspects, the cancer cell is a circulating tumor cell (CTC). In some aspects, the cell is a fibroblast cell, such as a primary fibroblast or newborn human foreskin fibroblast (Nuff cell). In some aspects, the cell is an immortalized cell line cell, such as a HEK293 cell or a CHO cell. In some aspects, the cell is a skin cell. In some aspects, the cell is a reproductive cell such as an oocyte, ovum, or zygote. In some aspects, the cell is a cluster of cells, such as an embryo, given that the cluster of cells is not disrupted when passing through the pore.

### VI. Compositions

In some aspects, the present disclosure further comprises a composition comprising any of the polynucleotides, vectors, or cells described herein. In some aspects, the composition is a pharmaceutical composition. Accordingly, disclosed herein is a pharmaceutical composition comprising (i) a polynucleotide described herein (e.g., comprising at least a first coding region and a second coding region, wherein the first coding region and the second coding region are not the same, and wherein the first coding region and the second coding region are linked), and (ii) a pharmaceutically acceptable carrier. In some aspects, provided herein is a pharmaceutical composition comprising (i) a cell which has been modified to comprise any of the polynucleotides described herein, and (ii) a pharmaceutically acceptable carrier. In some aspects, provided herein is a pharmaceutical composition comprising (i) a vector comprising any of the polynucleotides described herein, and (ii) a pharmaceutically acceptable carrier.

The terms “excipient” and “carrier” are used interchangeably and refer to an inert substance added to a pharmaceutical composition to further facilitate administration of a compound, e.g., any of the polynucleotides, vectors, or cells described herein. The terms “pharmaceutically acceptable carrier,” “pharmaceutically acceptable excipient,” and grammatical variations thereof, encompass any of the agents approved by a regulatory agency of the U.S. Federal government or listed in the U.S. Pharmacopeia for use in animals, including humans, as well as any carrier or diluent that does not cause the production of undesirable physiological effects to a degree that prohibits administration of the composition to a subject and does not abrogate the biological activity and properties of the administered compound. Included are excipients and carriers that are useful in preparing a pharmaceutical composition and are generally safe, non-toxic, and desirable.

Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol, salt-forming counter-ions such as sodium, metal complexes (e.g., Zn-protein complexes); and/or non-ionic surfactants such as TWEEN®, PLURONICS® or polyethylene glycol (PEG).

A pharmaceutical composition can be formulated for any route of administration to a subject. Specific examples of routes of administration include intramuscularly, subcutaneously, ophthalmic, intravenously, intraperitoneally, intradermally, intraorbitally, intracerebrally, intracranially, intraspinally, intraventricularly, intrathecally, intracistemally, intracapsularly, or intratumorally. Parenteral administration, characterized by either subcutaneous, intramuscular or intravenous injection, is also contemplated herein. Injectables can be prepared in conventional forms, either as liquid solutions or suspensions, solid forms suitable for solution or suspension in liquid prior to injection, or as emulsions. The injectables, solutions and emulsions also contain one or more excipients. Suitable excipients are, for example, water, saline, dextrose, or glycerol. In addition, if desired, the pharmaceutical compositions to be administered can also contain minor amounts of non-toxic auxiliary substances s, pH buffering agents, stabilizers, and other such agents, such as for example, sodium acetate, sorbitan monolaurate, triethanolamine oleate, and cyclodextrins.

### VII. Kits

Also disclosed herein are kits comprising any of the polynucleotides, vectors, compositions, or cells described herein. In some aspects, the kit is for use in immunotherapy against a disease or disorder (e.g., cancer) and/or treating or reducing the risk for the disease or disorder (e.g., cancer). In some aspects, the kit includes one or more containers comprising any of the polynucleotides, vectors, compositions, or cells described herein.

In some aspects, the kit comprises instructions for use in accordance with any of the methods described herein. For example, the included instructions can comprise a description of administration of the pharmaceutical composition described herein to treat, delay the onset, or alleviate a target disease. In some aspects, the instructions comprise a description of administering the composition described herein to a subject at risk of the target disease/disorder (e.g., cancer).

In some aspects, the instructions comprise dosage information, dosing schedule, and route of administration. In some aspects, the containers are unit doses, bulk packages (e.g., multi-dose packages) or sub-unit doses. In some aspects, the instructions are written instructions on a label or package insert (e.g., a paper sheet included in the kit). In some aspects, the instructions are machine-readable instructions (e.g., instructions carried on a magnetic or optical storage disk).

In some aspects, the label or package insert indicates that the composition disclosed herein is used for treating, delaying the onset, and/or alleviating a disease or disorder associated with cancer, such as those described herein. Instructions can be provided for practicing any of the methods described herein.

In some aspects, the kits described herein are in suitable packaging. In some aspects, suitable packing comprises vials, bottles, jars, flexible packaging (e.g., seal Mylar or plastic bags), or combinations thereof. In some aspects, the packaging comprises packages for use in combination with a specific device such as an inhaler, nasal administration device (e.g., an atomizer), or an infusion device such as a minipump. In some aspects, the kit comprises a sterile access port (for example the container can be an intravenous solution bag or a vial having a stopper pierceable by a hypodermic injection needle). In some aspects, the container can also have a sterile access port (for example the container can be an intravenous solution bag or a vial having a stopper pierceable by a hypodermic injection needle).

In some aspects, the kits further comprise additional components such as buffers and interpretive information. In some aspects, the kit comprises a container and a label or package insert(s) on or associated with the container. In some aspects, the disclosure provides articles of manufacture comprising the contents of the kits described herein.

### VIII. Uses and Methods

### Expression of Multiple Antigens

As is apparent from the present disclosure, the polynucleotides, vectors, cells, and/or pharmaceutical compositions described herein have numerous in vitro and in vivo utilities. For example, a polynucleotide described herein can be administered to cells in culture, in vitro or ex vivo, or to human subjects, e.g., in vivo, to induce expression of multiple antigens in a cell, which, in some aspects, can help treat a wide-range of diseases or disorders.

Accordingly, some aspects of the present disclosure is related to a method of inducing the expression of multiple antigens in a cell, comprising intracellularly delivering a polynucleotide described herein to the cell. As described and demonstrated herein, the multiple antigens are expressed in the cell simultaneously. For instance, in some aspects, provided herein is a method of inducing the expression of a first antigen and a second antigen in a cell, comprising intracellularly delivering a polynucleotide to the cell, wherein the polynucleotide comprises a first coding region encoding the first antigen and a second coding region encoding the second antigen, wherein the first coding region and the second coding region are not the same, and wherein the first coding region and the second coding region are linked. In some aspects, the first antigen and the second antigen are simultaneously expressed in the cell.

As described herein, in some aspects, the cell does not express the multiple antigens (e.g., first antigen and the second antigen) prior to the introduction of the polynucleotide into the cell. In some aspects, the cell expresses one or more of the multiple antigens prior to the introduction of the polynucleotide, but the expression is further increased after the introduction of the polynucleotide. Under either scenario, after the introduction of the polynucleotide described herein, the expression of the multiple antigens is increased as compared to a reference expression. In some aspects, the reference expression comprises the expression in the cell prior to the introduction of the polynucleotide. In some aspects, the reference expression comprises the expression in a corresponding cell that was not modified to comprise a polynucleotide described herein. In some aspects, the reference expression comprises the expression in a corresponding cell that was modified to comprise multiple polynucleotides, wherein each of the multiple polynucleotides encodes a separate antigen. In some aspects, after the introduction of the polynucleotide described herein, the expression of the multiple antigens is increased by at least about 10%, at least about 20%, at least about 30%, at least about 40%, at least about 50%, at least about 60%, at least about 70%, at least about 80%, at least about 90%, or at least about 100%, as compared to the reference expression. In some aspects, after the introduction of the polynucleotide described herein, the expression of the multiple antigens is increased by at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 35-fold, at least about 40-fold, at least about 45-fold, or at last about 50-fold or more, as compared to the reference expression.

In some aspects, intracellularly delivering the polynucleotide to the cell comprises passing a cell suspension comprising the cell through a constriction under a set of parameters, thereby causing a perturbation within the cell such that the polynucleotide enters the cell through the perturbation when contacted with the cell. In some aspects, the method further comprises contacting the cell with the polynucleotide. As used herein, “contacting” between a cell and a polynucleotide described herein does not require that the cell and the polynucleotide be in physical contact. As is apparent from the present disclosure, contacting between a cell and a polynucleotide occurs as long as the polynucleotide is capable of entering the cell once there are perturbations within the cell membrane of the cell. To help illustrate, in some aspects, a cell and a polynucleotide are in contact if they are both present within the same cell suspension, regardless of whether the cell and the polynucleotide are in physical contact or not. Accordingly, in some aspects, contacting the cell with the polynucleotide comprises incubating the cell suspension comprising the cell with the polynucleotide.

In some aspects, a polynucleotide described herein (e.g., comprising multiple coding regions that are linked) can be intracellularly delivered to a cell alone or in combination with one or more additional cargo (also referred to herein as “payload”). For instance, in some aspects, the additional cargo can comprise a separate polynucleotide. In some aspects, the separate polynucleotide can encode for a compound that improves and/or enhances the therapeutic effect of the polynucleotides described herein (e.g., comprising multiple coding regions that are linked). As is generally understood in the art, for optimal T cell activation, multiple signals are required: (1) “signal 1”: antigen-specific signal provided by the binding of the TCR to antigenic peptide complexed with MHC; (2) “signal 2”: mediated by the engagement of co-stimulatory molecules such as CD80 and CD86 on antigen-presenting cells (APC); and (3) “signal 3”: mediated by cytokines (e.g., IL-2 and/or IL-12). Accordingly, in some aspects, a polynucleotide described herein (e.g., comprising multiple coding regions that are linked) is intracellularly delivered to a cell in combination with one or more separate polynucleotides, wherein the one or more separate polynucleotides encode a co-stimulatory molecule (i.e., signal 2) and/or a cytokine (i.e., signal 3).

In some aspects, where multiple polynucleotides are being delivered, they can be delivered to a cell using a single squeeze processing e.g., a cell suspension comprises the multiple polynucleotides, which are delivered to the cell in combination; “concurrent delivery”). In some aspects, the multiple polynucleotides (e.g., polynucleotide described herein and a separate polynucleotide encoding for a co-stimulatory molecule and/or cytokine) can be delivered to a cell sequentially. As used herein, the term “sequential delivery” refers to the delivery of multiple polynucleotides to a cell, where a first polynucleotide (e.g., comprising multiple coding regions that are linked) is delivered to the cell and then the second (or subsequent) polynucleotide (e.g., a separate polynucleotide encoding a co-stimulatory molecule and/or cytokine) is delivered to the cell. In some aspects, the first polynucleotide, the second polynucleotide, or both the first and second polynucleotides can be delivered to the cell using squeeze processing. For instance, in some aspects, the first polynucleotide can be delivered to the cell using squeeze processing, and the second polynucleotide can be delivered to the cell using non-squeeze processing (e.g., transfection). In some aspects, the first polynucleotide can be delivered to the cell using non-squeeze processing (e.g., transfection), and the second polynucleotide can be delivered to the cell using squeeze processing. In some aspects, the first polynucleotide can be delivered to the cell using a first squeeze, and then the second polynucleotide can be delivered to the cell using a second squeeze (also referred to herein as “sequential squeeze” or “sequential squeeze processing”). Accordingly, sequential delivery useful for the present disclosure can comprise multiple squeeze processing. In some aspects, each of the multiple squeeze processing delivers a separate polynucleotide to the cell. In some aspects, one or more of the multiple squeeze processing do not involve the delivery of a polynucleotide. For instance, in some aspects, a sequential delivery method described herein comprises a first squeeze, a second squeeze, and a third squeeze, wherein the first squeeze comprises passing a cell without any payload through a first constriction, the second squeeze comprises passing the cell from the first squeeze through a second constriction to deliver a first polynucleotide (e.g., comprising multiple coding regions that are linked) to the cell, and the third squeeze comprises passing the cell from the second squeeze through a third constriction to deliver a second polynucleotide (e.g., encoding a co-stimulatory molecule and/or cytokine) to the cell. Not to be bound by any one theory, in some aspects, passing the cell through the first constriction without any payload (i.e., the first squeeze) can help prepare the cell for subsequent deliveries, e.g., can improve the delivery efficiency of the first polynucleotide and/or the second polynucleotide.

In some aspects, a combination of payloads (e.g., polynucleotides) can be delivered to a cell (e.g., stem cells or PBMCs) repeatedly. For instance, in some aspects, a combination of polynucleotides (e.g., a first polynucleotide comprising multiple coding regions that are linked and a second polynucleotide encoding a co-stimulatory molecule and/or cytokine) is delivered to cells with a first squeeze processing; then, the combination of the polynucleotides is delivered to the cells again with a second squeeze processing. In some aspects, the first squeeze processing includes a microfluidic device (e.g., chip) with multiple rows of constrictions, such that the squeeze process occurs on a single microfluidic device (e.g., chip). As further described herein, in some aspects, the second squeeze processing can occur immediately after the cells have gone through the first squeeze processing (e.g., immediately after the cells pass through the constriction of the first squeeze processing). In some aspects, the second squeeze processing can occur after some time after the first squeeze processing (e.g., at least about 1 minute, at least about 30 minutes, at least about 1 hour, at least about 6 hours, at least about 12 hours, or at least about 1 day after the cells pass through the constriction of the first squeeze processing).

### Constrictions

### Microfluidic Channels

As described herein, a constriction is used to cause a physical deformity in the cells, such that perturbations are created within the cell membrane of the cells, allowing for the delivery of a payload (e.g., polynucleotide described herein comprising multiple coding regions that are linked) into the cell. In some aspects, a constriction is within a channel contained within a microfluidic device (referred to herein as “microfluidic channel” or “channel”). Where multiple channels are involved, in some aspects, the multiple channels can be placed in parallel and/or in series within the microfluidic device. In some aspects, the cells described herein can be passed through at least about 2, at least about 3, at least about 4, at least about 5, at least about 6, at least about 7, at least about 8, at least about 9, at least about 10, at least about 20, at least about 30, at least about 40, at least about 50, at least about 75, at least about 100, at least about 150, at least about 200, at least about 250, at least about 300, at least about 350, at least about 400, at least about 450, at least about 500, at least about 550, at least about 600, at least about 650, at least about 700, at least about 750, at least about 800, at least about 850, at least about 900, at least about 950, at least about 1,000 or more separate constrictions. In some aspects, the cells described herein are passed through more than about 1,000 separate constrictions. In some aspects, the multiple constrictions can be part of a single microfluidic device (e.g., multi-row constriction chip). In some aspects, one or more of the multiple constrictions can be part of different microfluidic devices. For instance, in some aspects, the cells described herein (e.g., stem cells or PBMCs) undergo a first squeeze processing, in which the cells pass through a first constriction in a first microfluidic device (e.g., chip). Then, after the cells have undergone the first squeeze processing (e.g., passed through the first constriction), the cells undergo a second squeeze processing, in which the cells pass through a second constriction in a second microfluidic device (e.g., chip). In some aspects, each of the constrictions are the same (e.g., has the same length, width, and/or depth). In some aspects, one or more of the constrictions are different. Where plurality of constrictions are used, the plurality of constrictions can comprise a first constriction which is associated with a first polynucleotide (e.g., comprising multiple coding regions that are linked), and a second constriction which is associated with a second polynucleotide (e.g., encoding a co-stimulatory molecule and/or cytokine), wherein the cell suspension passes through the first constriction such that the first polynucleotide is delivered to one or more cells of the plurality of cells, and then the cell suspension passes through the second constriction such that the second polynucleotide is delivered to the one or more cells of the plurality of cells. In some aspects, the cell suspension is passed through the second constriction at least about 1 minute, at least about 30 minutes, at least about 1 hour, at least about 6 hours, at least about 12 hours, or at least about 1 day after the cell suspension is passed through the first constriction.

In some aspects, where the cell suspension is passed through multiple constrictions (e.g., multiple squeeze processing), the cells remain viable after passing through each constriction.

As is apparent from the present disclosure, in some aspects, multiple constrictions can comprise two or more constrictions present within a single microfluidic device (e.g., multi-row constriction chip), such the cells pass through the multiple constrictions sequentially. In some aspects, the multiple constrictions are part of separate microfluidic devices, such that a first constriction is associated with a first microfluidic device and a second constriction is associated with a second microfluidic device. For instance, in some aspects, cells are passed through a first constriction (i.e., first squeeze processing), which is associated with a first microfluidic device (e.g., chip). After the cells have passed through the first constriction, the cells are passed through a second constriction (i.e., second squeeze processing), which is associated with a second microfluidic device (e.g., chip). In some aspects, after passing through the first constriction, the cells are cultured in a medium prior to passing the cells through the second constriction. In some aspects, the cells are cultured for at least about 1 minute, at least about 30 minutes, at least about 1 hour, at least about 6 hours, at least about 12 hours, or at least about 1 day before passing the cells through the second constriction. As is apparent from the present disclosure, in some aspects, the first and second constrictions have the same length, depth, and/or width. In some aspects, the first and second constrictions can have different length, depth, and/or width.

In some aspects, after passing through a constriction, at least about 50%, at least about 55%, at least about 60%, at least about 65%, at least about 70%, at least about 75%, at least about 80%, at least about 85%, at least about 90%, at least about 95%, or about 100% of the cells remain viable. Where the cells pass through multiple constrictions (e.g., part of a single microfluidic device or separate microfluidic devices), at least about 50%, at least about 55%, at least about 60%, at least about 65%, at least about 70%, at least about 75%, at least about 80%, at least about 85%, at least about 90%, at least about 95%, or about 100% of the cells remain viable after passing through each of the multiple constrictions. The viability of the cells can be measured using any suitable methods known in the art. In some aspects, the viability of the cells can be measured using a Nucleocounter NC-200, an Orflo Moxi Go II Cell Counter, or both.

Exemplary microfluidic channels containing cell-deforming constrictions for use in the methods disclosed herein are described in US Publ. No. 2020/0277566 A1, US Publ. No. 2020/0332243 A1, US Publ. No. 2020/0316604 A1, U.S. Provisional Appl. No. 63/131,423, and U.S. Provisional Appl. No. 63/131,430, each of which is incorporated herein by reference in its entirety.

In some aspects, a microfluidic channel described herein (i.e., comprising a constriction) includes a lumen and is configured such that a cell suspended in a buffer (e.g., cell suspension) can pass through the channel. Microfluidic channels useful for the present disclosure can be made using any suitable materials available in the art, including, but not limited to, silicon, metal (e.g., stainless steel), plastic (e.g., polystyrene), ceramics, glass, crystalline substrates, amorphous substrates, polymers (e.g., Poly-methyl methacrylate (PMMA), PDMS, Cyclic Olefin Copolymer (COC)), or combinations thereof. In some aspects, the material is silicon. Fabrication of the microfluidic channel can be performed by any method known in the art, including, but not limited to, dry etching for example deep reactive ion etching, wet etching, photolithography, injection molding, laser ablation, SU-8 masks, or combinations thereof. In some aspects, the fabrication is performed using dry etching.

In some aspects, a microfluidic channel useful for the present disclosure comprises an entrance portion, a center point, and an exit portion. In some aspects, the cross-section of one or more of the entrance portion, the center point, and/or the exit portion can vary. For example, the cross-section can be circular, elliptical, an elongated slit, square, hexagonal, or triangular in shape.

The entrance portion defines a constriction angle. In some aspects, by modulating (e.g., increasing or decreasing) the constriction angle, any clogging of the constriction can be reduced or prevented. In some aspects, the angle of the exit portion can also be modulated. For example, in some aspects, the angle of the exit portion can be configured to reduce the likelihood of turbulence that can result in non-laminar flow. In some aspects, the walls of the entrance portion and/or the exit portion are linear. In some aspects, the walls of the entrance portion and/or the exit portion are curved.

In some aspects, the length, depth, and/or width of the constriction can vary. In some aspects, by modulating (e.g., increasing or decreasing) the length, depth, and/or width of the constriction, the delivery efficiency of a payload can be regulated. As used herein, the term “delivery efficiency” refers to the amount of payload that is delivered into the cell. For instance, an increased delivery efficiency can occur when the total amount of payload that is delivered is increased.

In some aspects, the constriction has a length of less than about 1 μm. In some aspects the constriction has a length of about 0 μm to about 100 μm. In some aspects, the length of the constriction is less than about 0.1 μm, less than about 0.2 μm, less than about 0.3 μm, less than about 0.4 μm, less than about 0.5 μm, less than about 0.6 μm, less than about 0.7 μm, less than about 0.8 μm, less than about 0.9 μm, less than about 1 μm, less than about 2.5 μm, less than about 5 μm, less than about 7.5 μm, less than about 10 μm, less than about 12.5 μm, less than about 15 μm, less than about 20 μm, less than about 30 μm, less than about 40 μm, less than about 50 μm, less than about 60 μm, less than about 70 μm, less than about 80 μm, less than about 90 μm, or less than about 100 μm. In some aspects, the length of the constriction is about 0.1 μm, about 0.2 μm, about 0.3 μm, about 0.4 μm, about 0.5 μm, about 0.6 μm, about 0.7 μm, about 0.8 μm, about 0.9 μm, about 1 μm, about 2.5 μm, about 5 μm, about 7.5 μm, about 10 μm, about 12.5 μm, about 15 μm, about 20 μm, about 30 μm, about 40 μm, about 50 μm, about 60 μm, about 70 μm, about 80 μm, about 90 μm, or about 100 μm. In some aspects, the length of the constriction is about 10 μm. In some aspects, the constriction has a length of about 0 μm. For example, in some aspects, a microfluidic device (e.g., chip) useful for the present disclosure comprises a constriction that resembles two points of a diamond coming together, such that the length of the constriction is about 0 μm.

In some aspects, the width of the constriction is between about 0 μm to about 10 μm. In some aspects, the width of the constriction is less than about 0.1 μm, less than about 0.2 μm, less than about 0.3 μm, less than about 0.4 μm, less than about 0.5 μm, less than about 0.6 μm, less than about 0.7 μm, less than about 0.8 μm, less than about 0.9 μm, less than about 1 μm, less than about 2 μm, less than about 3 μm, less than about 4 μm, less than about 5 μm, less than about 6 μm, less than about 7 μm, less than about 8 μm, less than about 9 μm, or less than about 10 μm. In some aspects, the width of the constriction is about 0.1 μm, about 0.2 μm, about 0.3 μm, about 0.4 μm, about 0.5 μm, about 0.6 μm, about 0.7 μm, about 0.8 μm, about 0.9 μm, about 1 μm, about 2 μm, about 3 μm, about 4 μm, about 5 μm, about 6 μm, about 7 μm, about 8 μm, about 9 μm, or about 10 μm. In some aspects, width of the constriction is between about 3 μm to about 10 μm. In some aspects, the width of the constriction is about 6 μm.

In some aspects, the depth of the constriction is at least about 1 μm. In some aspects, the depth of the constriction is at least about 2 μm, at least about 3 μm, at least about 4 μm, at least about 5 μm, at least about 10 μm, at least about 20 μm, at least about 30 μm, at least about 40 μm, at least about 50 μm, at least about 60 μm, at least about 70 μm, at least about 80 μm, at least about 90 μm, at least about 100 μm, at least about 110 μm, or at least about 120 μm. In some aspects, the depth of the constriction is about 5 μm to about 90 μm. In some aspects, the depth of the constriction is about 5 μm, about 10 μm, about 20 μm, about 30 μm, about 40 μm, about 50 μm, about 60 μm, about 70 μm, about 80 μm, or about 90 μm. In some aspects, the depth of the constriction is about 70 μm.

In some aspects, the length of the constriction is about 10 μm, the width of the constriction is about 6 μm, and the depth of the constriction is about 70 μm. In some aspects, the length of the constriction is 10 μm, the width of the constriction is 6 μm, and the depth of the constriction is 70 μm.

In some aspects, the diameter of a constriction (e.g., contained within a microfluidic channel) is a function of the diameter of one or more cells that are passed through the constriction. Not to be bound by any one theory, in some aspects, the diameter of the constriction is less than that of the cells, such that a deforming force is applied to the cells as they pass through the constriction, resulting in the transient physical deformity of the cells.

Accordingly, in some aspects, the diameter of the constriction (also referred to herein as “constriction size”) is about 20% to about 99% of the diameter of the cell. In some aspects, the constriction size is about 20%, about 30%, about 40%, about 50%, about 60%, about 70%, about 80%, about 90%, or about 99% of the cell diameter. As is apparent from the present disclosure, by modulating (e.g., increasing or decreasing) the diameter of a constriction, the delivery efficiency of a payload into a cell can also be regulated.

### Surface Having Pores

In some aspects, a constriction described herein comprises a pore, which is contained in a surface. Non-limiting examples of pores contained in a surface that can be used with the present disclosure are described in, e.g., US Publ. No. 2019/0382796 A1, which is incorporated herein by reference in its entirety.

In some aspects, a surface useful for the present disclosure (i.e., comprising one or more pores that can cause a physical deformity in a cell as it passes through the pore) can be made using any suitable materials available in the art and/or take any one of a number of forms. Non-limiting examples of such materials include synthetic or natural polymers, polycarbonate, silicon, glass, metal, alloy, cellulose nitrate, silver, cellulose acetate, nylon, polyester, polyethersulfone, polyacrylonitrile (PAN), polypropylene, PVDF, polytetrafluorethylene, mixed cellulose ester, porcelain, ceramic, or combinations thereof.

In some aspects, the surface comprises a filter. In some aspects, the filter is a tangential flow filter. In some aspects, the surface comprises a membrane. In some aspects, the surface comprises a sponge or sponge-like matrix. In some aspects, the surface comprises a matrix. In some aspects, the surface comprises a tortuous path surface. In some aspects, the tortuous path surface comprises cellulose acetate.

The surface disclosed herein (i.e., comprising one or more pores) can have any suitable shape known in the art. Where the surface has a 2-dimensional shape, the surface can be, without limitation, circular, elliptical, round, square, star-shaped, triangular, polygonal, pentagonal, hexagonal, heptagonal, or octagonal. In some aspects, the surface is round in shape. Where the surface has a 3-dimensional shape, in some aspects, the surface can be, without limitation, cylindrical, conical, or cuboidal.

As is apparent from the present disclosure, a surface that is useful for the present disclosure (e.g., comprising one or more pores) can have various cross-sectional widths and thicknesses. In some aspects, the cross-sectional width of the surface is between about 1 mm and about 1 m. In some aspects, the surface has a defined thickness. In some aspects, the surface thickness is uniform. In some aspects, the surface thickness is variable. For example, in some aspects, certain portions of the surface are thicker or thinner than other portions of the surface. In such aspects, the thickness of the different portions of the surface can vary by about 1% to about 90%. In some aspects, the surface is between about 0.01 μm to about 5 mm in thickness.

The cross-sectional width of the pores can depend on the type of cell that is being targeted with a payload. In some aspects, the pore size is a function of the diameter of the cell of cluster of cells to be targeted. In some aspects, the pore size is such that a cell is perturbed (i.e., physically deformed) upon passing through the pore. In some aspects, the pore size is less than the diameter of the cell. In some aspects, the pore size is about 20% to about 99% of the diameter of the cell. In some aspects, the pore size is about 20%, about 30%, about 40%, about 50%, about 60%, about 70%, about 80%, about 90%, or about 99% of the diameter of the cell. In some aspects, the pore size is about 0.4 μm, about 0.5 μm, about 0.6 μm, about 0.7 μm, about 0.8 μm, about 0.9 μm, about 1 μm, about 2 μm, about 3 μm, about 4 μm, about 5 μm, about 6 μm, about 7 μm, about μm, about 9 μm, about 10 μm, about 11 μm, about 12 μm, about 13 μm, about 14 μm, or about 15 μm or more.

The entrances and exits of a pore can have a variety of angles. In some aspects, by modulating (e.g., increasing or decreasing) the pore angle, any clogging of the pore can be reduced or prevented. In some aspects, the flow rate (i.e., the rate at which a cell or a suspension comprising the cell passes through the pore) is between about 0.001 mL/cm/sec to about 100 L/cm/sec. For example, the angle of the entrance or exit portion can be between about 0 and about 90 degrees. In some aspects, the pores have identical entrance and exit angles. In some aspects, the pores have different entrance and exit angles. In some aspects, the pore edge is smooth, e.g., rounded or curved. As used herein, a “smooth” pore edge has a continuous, flat, and even surface without bumps, ridges, or uneven parts. In some aspects, the pore edge is sharp. As used herein, a “sharp” pore edge has a thin edge that is pointed or at an acute angle. In some aspects, the pore passage is straight. As used herein, a “straight” pore passage does not contain curves, bends, angles, or other irregularities. In some aspects, the pore passage is curved. As used herein, a “curved” pore passage is bent or deviates from a straight line. In some aspects, the pore passage has multiple curves, e.g., about 2, about 3, about 4, about 5, about 6, about 7, about 8, about 9, about 10 or more curves.

The pores can have any shape known in the art, including a 2-dimensional or 3-dimensional shape. The pore shape (e.g., the cross-sectional shape) can be, without limitation, circular, elliptical, round, square, star-shaped, triangular, polygonal, pentagonal, hexagonal, heptagonal, and octagonal. In some aspects, the cross-section of the pore is round in shape. In some aspects, the 3-dimensional shape of the pore is cylindrical or conical. In some aspects, the pore has a fluted entrance and exit shape. In some aspects, the pore shape is homogenous (i.e., consistent or regular) among pores within a given surface. In some aspects, the pore shape is heterogeneous (i.e., mixed or varied) among pores within a given surface.

A surface useful for the present disclosure can have a single pore. In some aspects, a surface useful for the present disclosure comprises multiple pores. In some aspects, the pores encompass about 10% to about 80% of the total surface area of the surface. In some aspects, the surface contains about 1.0×10⁵ to about 1.0×10³⁰ total pores. In some aspects, the surface comprises between about 10 and about 1.0×10¹⁵ pores per mm² surface area.

The pores can be distributed in numerous ways within a given surface. In some aspects, the pores are distributed in parallel within a given surface. In some aspects, the pores are distributed side-by-side in the same direction and are the same distance apart within a given surface. In some aspects, the distribution of the pores is ordered or homogeneous. In such aspects, the pores can be distributed in a regular, systematic pattern, or can be the same distance apart within a given surface. In some aspects, the distribution of the pores is random or heterogeneous. For instance, in some aspects, the pores are distributed in an irregular, disordered pattern, or are different distances apart within a given surface.

In some aspects, multiple surfaces are used, such that a cell passes through multiple pores, wherein the pores are on different surfaces. In some aspects, multiple surfaces are distributed in series. The multiple surfaces can be homogeneous or heterogeneous in surface size, shape, and/or roughness. The multiple surfaces can further contain pores with homogeneous or heterogeneous pore size, shape, and/or number, thereby enabling the simultaneous delivery of a range of payloads into different cell types.

In some aspects, an individual pore, e.g., of a surface that can be used with the present disclosure, has a uniform width dimension (i.e., constant width along the length of the pore passage). In some aspects, an individual pore has a variable width (i.e., increasing or decreasing width along the length of the pore passage). In some aspects, pores within a given surface have the same individual pore depths. In some aspects, pores within a given surface have different individual pore depths. In some aspects, the pores are immediately adjacent to each other. In some aspects, the pores are separated from each other by a distance. In some aspects, the pores are separated from each other by a distance of about 0.001 μm to about 30 mm.

In some aspects, the surface is coated with a material. The material can be selected from any material known in the art, including, without limitation, Teflon, an adhesive coating, surfactants, proteins, adhesion molecules, antibodies, anticoagulants, factors that modulate cellular function, nucleic acids, lipids, carbohydrates, transmembrane proteins, or combinations thereof. In some aspects, the surface is coated with polyvinylpyrrolidone. In some aspects, the material is covalently attached to the surface. In some aspects, the material is non-covalently attached to the surface. In some aspects, the surface molecules are released at the cells pass through the pores.

In some aspects, the surface has modified chemical properties. In some aspects, the surface is hydrophilic. In some aspects, the surface is hydrophobic. In some aspects, the surface is charged. In some aspects, the surface is positively and/or negatively charged. In some aspects, the surface can be positively charged in some regions and negatively charged in other regions. In some aspects, the surface has an overall positive or overall negative charge. In some aspects, the surface can be any one of smooth, electropolished, rough, or plasma treated. In some aspects, the surface comprises a zwitterion or dipolar compound. In some aspects, the surface is plasma treated.

In some aspects, the surface is contained within a larger module. In some aspects, the surface is contained within a syringe, such as a plastic or glass syringe. In some aspects, the surface is contained within a plastic filter holder. In some aspects, the surface is contained within a pipette tip.

### Cell Perturbation

As described herein, as a cell passes through a constriction, it becomes physically deformed, such that there is a perturbation (e.g., a hole, tear, cavity, aperture, pore, break, gap, perforation) in the cell membrane of the cell. Such perturbation in the cell membrane is temporary and sufficient for any of the payloads (e.g., polynucleotide comprising multiple coding regions that are linked) described herein to be delivered into the cell. Cells have self-repair mechanisms that allow the cells to repair any disruption in their cell membrane. See Blazek et al., Physiology (Bethesda) 30 (6): 438-48 (November 2015), which is incorporated herein by reference in its entirety. Accordingly, in some aspects, once the cells have passed through the constriction (e.g., microfluidic channel or pores), the perturbations in the cell membrane can be reduced or eliminated, such that the payload that was delivered into the cell does not exit the cell.

In some aspects, the perturbation in the cell membrane lasts from about 1.0×10⁻⁹ seconds to about 2 hours after the pressure is removed (e.g., cells have passed through the constriction). In some aspects, the cell perturbation lasts for about 1.0×10⁻⁹ second to about 1 second, for about 1 second to about 1 minute, or for about 1 minute to about 1 hour. In some aspects, the cell perturbation lasts for between about 1.0×10⁻⁹ second to about 1.0×10⁻¹ second, between about 1.0×10⁻⁹ second to about 1.0×10⁻² second, between about 1.0×10⁻⁹ second to about 1.0×10⁻³ second, between about 1.0×10⁻⁹ second to about 1.0×10⁻⁴ second, between about 1.0×10⁻⁹ second to about 1.0×10⁻⁵ second, between about 1.0×10⁻⁹ second to about 1.0×10⁻⁶ second, between about 1.0×10⁻⁹ second to about 1.0×10⁻⁷ second, or between about 1.0×10⁻⁹ second to about 1.0×10⁻⁸ second. In some aspects, the cell perturbation lasts for about 1.0×10⁻⁸ second to about 1.0×10⁻¹ second, for about 1.0×10⁻⁷ second to about 1.0×10⁻¹ second, about 1.0×10⁻⁶ second to about 1.0×10⁻¹ second, about 1.0×10⁻⁵ second to about 1.0×10⁻¹ second, about 1.0×10⁻⁴ second to about 1.0×10⁻¹ second, about 1.0×10⁻³ second to about 1.0×10⁻¹ second, or about 1.0×10⁻² second to about 1.0×10⁻¹ second. The cell perturbations (e.g., pores or holes) created by the methods described herein are not formed as a result of assembly of polypeptide subunits to form a multimeric pore structure such as that created by complement or bacterial hemolysins.

In some aspects, as the cell passes through the constriction, the pressure applied to the cells temporarily imparts injury to the cell membrane that causes passive diffusion of material through the perturbation. In some aspects, the cell is only deformed or perturbed for a brief period of time, e.g., on the order of 100 μs or less to minimize the chance of activating apoptotic pathways through cell signaling mechanisms, although other durations are possible (e.g., ranging from nanoseconds to hours). In some aspects, the cell is deformed for less than about 1.0×10⁻⁹ second to less than about 2 hours. In some aspects, the cell is deformed for less than about 1.0×10⁻⁹ second to less than about 1 second, less than about 1 second to less than about 1 minute, or less than about 1 minute to less than about 1 hour. In some aspects, the cell is deformed for about 1.0×10⁻⁹ second to about 2 hours. In some aspects, the cell is deformed for about 1.0×10⁻⁹ second to about 1 second, about 1 second to about 1 minute, or about 1 minute to about 1 hour. In some aspects, the cell is deformed for between any one of about 1.0×10⁻⁹ second to about 1.0×10⁻¹ second, about 1.0×10⁻⁹ second to about 1.0×10⁻² second, about 1.0×10⁻⁹ second to about 1.0×10⁻³ second, about 1.0×10⁻⁹ second to about 1.0×10⁻⁴ second, about 1.0×10⁻⁹ second to about 1.0×10⁻⁵ second, about 1.0×10⁻⁹ second to about 1.0×10⁻⁶ second, about 1.0×10⁻⁹ second to about 1.0×10⁻⁷ second, or about 1.0×10⁻⁹ second to about 1.0×10⁻⁸ second. In some aspects, the cell is deformed or perturbed for about 1.0×10⁻⁸ second to about 1.0×10⁻¹ second, for about 1.0×10⁻⁷ second to about 1.0×10⁻¹ second, about 1.0×10⁻⁶ second to about 1.0×10⁻¹ second, about 1.0×10⁻⁵ second to about 1.0×10⁻¹ second, about 1.0×10⁻⁴ second to about 1.0×10⁻¹ second, about 1.0×10⁻³ second to about 1.0×10⁻¹ second, or about 1.0×10⁻² second to about 1.0×10⁻¹ second. In some aspects, deforming the cell includes deforming the cell for a time ranging from, without limitation, about 1 μs to at least about 750 μs, e.g., at least about 1 μs, at least about 10 μs, at least about 50 μs, at least about 100 μs, at least about 500 μs, or at least about 750 μs.

In some aspects, the delivery of a polynucleotide described herein (e.g., comprising multiple coding regions that are linked) into the cell occurs simultaneously with the cell passing through the constriction. In some aspects, delivery of the polynucleotide into the cell can occur after the cell passes through the constriction (i.e., when perturbation of the cell membrane is still present and prior to cell membrane of the cells being restored). In some aspects, delivery of the polynucleotide into the cell occurs on the order of minutes after the cell passes through the constriction. In some aspects, a perturbation in the cell after it passes through the constriction is corrected within the order of about five minutes after the cell passes through the constriction.

In some aspects, the viability of a cell (e.g., stem cell or PBMC) after passing through a constriction is about 5% to about 100%. In some aspects, the cell viability after passing through the constriction is at least about 5%, at least about 10%, at least about 20%, at least about 30%, at least about 40%, at least about 50%, at least about 60%, at least about 70%, at least about 75%, at least about 80%, at least about 85%, at least about 90%, at least about 95%, or at least about 99%. In some aspects, the cell viability is measured from about 1.0×10⁻² second to at least about 10 days after the cell passes through the constriction. For example, the cell viability can be measured from about 1.0×10⁻² second to about 1 second, about 1 second to about 1 minute, about 1 minute to about 30 minutes, or about 30 minutes to about 2 hours after the cell passes through the constriction. In some aspects, the cell viability is measured about 1.0×10⁻² second to about 2 hours, about 1.0×10⁻² second to about 1 hour, about 1.0×10⁻² second to about 30 minutes, about 11.0×10⁻² second to about 1 minute, about 1.0×10⁻² second to about 30 seconds, about 1.0×10⁻² second to about 1 second, or about 1.0×10⁻² second to about 0.1 second after the cell passes through the constriction. In some aspects, the cell viability is measured about 1.5 hours to about 2 hours, about 1 hour to about 2 hours, about 30 minutes to about 2 hours, about 15 minutes to about 2 hours, about 1 minute to about 2 hours, about 30 seconds to about 2 hours, or about 1 second to about 2 hours after the cell passes through the constriction. In some aspects, the cell viability is measured about 2 hours to about 5 hours, about 5 hours to about 12 hours, about 12 hours to about 24 hours, or about 24 hours to about 10 days after the cell passes through the constriction.

### Delivery Parameters

As is apparent from the present disclosure, a number of parameters can influence the delivery efficiency of a polynucleotide described herein (e.g., comprising multiple coding regions that are linked) into a cell using the squeeze processing methods provided herein. Accordingly, by modulating (e.g., increasing or decreasing) one or more of the delivery parameters, the delivery of a payload into a cell can be improved. Therefore, in some aspects, the present disclosure relates to a method of increasing the delivery of a payload (e.g., polynucleotide described herein) into a cell, wherein the method comprises modulating one or more parameters under which a cell suspension is passed through a constriction, wherein the cell suspension comprises a population of the cells, and wherein the one or more parameters increase the delivery of a payload into one or more cells of the population of cells compared to a reference parameter. As described elsewhere in the present disclosure, the payload can be in contact with the population of cells before, during, or after the squeezing step.

In some aspects, by modulating one or more of the delivery parameters, the delivery of the payload (e.g., polynucleotide described herein) into the one or more cells is increased by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 40-fold, or at least about 50-fold, compared to a delivery of the payload agent into a corresponding cell using the reference parameter.

In some aspects, the one or more delivery parameters that can be modulated to increase the delivery efficiency of a parameter comprises a cell density (i.e., the concentration of the cells present, e.g., in the cell suspension), pressure, or both. Additional examples of delivery parameters that can be modulated are provided elsewhere in the present disclosure.

In some aspects, the cell density is about 1×10⁷ cells/mL, about 2×10⁷ cells/mL, about 3×10⁷ cells/mL, about 4×10⁷ cells/mL, about 5×10⁷ cells/mL, about 6×10⁷ cells/mL, about 7×10⁷ cells/mL, about 8×10⁷ cells/mL, about 9×10⁷ cells/mL, about 1×10⁸ cells/mL, about 1.1×10⁸ cells/mL, about 1.2×10⁸ cells/mL, about 1.3×10⁸ cells/mL, about 1.4×10⁸ cells/mL, about 1.5×10⁸ cells/mL, about 2.0×10⁸ cells/mL, about 3.0×10⁸ cells/mL, about 4.0×10⁸ cells/mL, about 5.0×10⁸ cells/mL, about 6.0×10⁸ cells/mL, about 7.0×10⁸ cells/mL, about 8.0×10⁸ cells/mL, about 9.0×10⁸ cells/mL, or about 1.0×10⁹ cells/mL or more. In some aspects, the cell density is between about 6×10⁷ cells/mL and about 1.2×10⁸ cells/mL.

In some aspects, the pressure is about 20 psi, about 25 psi, about 30 psi, about 35 psi, about 40 psi, about 50 psi, about 55 psi, about 60 psi, about 65 psi, about 70 psi, about 75 psi, about 80 psi, about 85 psi, about 90 psi, about 95 psi, about 100 psi, about 110 psi, about 120 psi, about 130 psi, about 140 psi, about 150 psi, about 160 psi, about 170 psi, about 180 psi, about 190 psi, or about 200 psi or more. In some aspects, the pressure is between about 30 psi and about 90 psi.

In some aspects, the particular type of device (e.g., microfluidic chip) can also have an effect on the delivery efficiency of a payload described herein (e.g., polynucleotide described herein). In the case of a microfluidic chip, different chips can have different constriction parameters, e.g., length, depth, and width of the constriction; entrance angle, exit angle, length, depth, and width of the approach region, etc. As described herein, such variables can influence the delivery of a payload into a cell using the squeeze processing methods of the present disclosure. In some aspects, the length of the constriction is up to 100 μm. For instance, in some aspects, the length is about 1 μm, about 5 μm, 10 μm, about 20 μm, about 30 μm, about 40 μm, about 50 μm, about 60 μm, about 70 μm, about 80 μm, about 90 μm, or about 100 μm. In some aspects, the length of the constriction is less than 1 μm. In some aspects, the length of the constriction is less than about 1 μm, less than about 5 μm, less than about 10 μm, less than about 20 μm, less than about 30 μm, less than about 40 μm, less than about 50 μm, less than about 60 μm, less than about 70 μm, less than about 80 μm, less than about 90 μm, or less than about 100 μm. In some aspects, the constriction has a length of about 10 μm.

In some aspects, the width of the constriction is up to about 10 μm. In some aspects, the width of the constriction is less than about 1 μm, less than about 2 μm, less than about 3 μm, less than about 4 μm, less than about 5 μm, less than about 6 μm, less than about 7 μm, less than about 8 μm, less than about 9 μm, or less than about 10 μm. In some aspects, the width is between about 3 μm to about 10 μm. In some aspects, the width is about 3 μm, about 4 μm, about 5 μm, about 6 μm, about 7 μm, about 8 μm, about 9 μm, or about 10 μm. In some aspects, the width of the constriction is about 6 μm.

In some aspects, the depth of the constriction is at least about 1 μm. In some aspects, the depth of the constriction is at least about 1 μm, at least about 2 μm, at least about 3 μm, at least about 4 μm, at least about 5 μm, at least about 10 μm, at least about 20 μm, at least about 30 μm, at least about 40 μm, at least about 50 μm, at least about 60 μm, at least about 70 μm, at least about 80 μm, at least about 90 μm, at least about 100 μm, at least about 110 μm, or at least about 120 μm. In some aspects, the depth is between about 5 μm to about 90 μm. In some aspects, the depth is about 5 μm, about 10 μm, about 15 μm, about 20 μm, about 30 μm, about 40 μm, about 50 μm, about 60 μm, about 70 μm, about 80 μm, or about 90 μm. In some aspects, the depth of the constriction is about 70 μm.

In some aspects, the length is about 10 μm, the width is about 6 μm, and depth is about 70 μm.

Additional examples of parameters that can influence the delivery of a payload into the cell include, but are not limited to, the dimensions of the constriction (e.g., length, width, and/or depth), the entrance angle of the constriction, the surface properties of the constrictions (e.g., roughness, chemical modification, hydrophilic, hydrophobic), the operating flow speeds, payload concentration, the amount of time that the cell recovers, or combinations thereof. Further parameters that can influence the delivery efficiency of a payload (e.g., polynucleotide described herein) can include the velocity of the cell in the constriction, the shear rate in the constriction, the viscosity of the cell suspension, the velocity component that is perpendicular to flow velocity, and time in the constriction. Such parameters can be designed to control delivery of the payload.

In some aspects, the temperature used in the methods of the present disclosure can also have an effect on the delivery efficiency of the payloads into the cell, as well as the viability of the cell. In some aspects, the squeeze processing method is performed between about −5° C. and about 45° C. For example, the methods can be carried out at room temperature (e.g., about 20° C.), physiological temperature (e.g., about 37° C.), higher than physiological temperature (e.g., greater than about 37° C. to 45° C. or more), or reduced temperature (e.g., about −5° C. to about 4° C.), or temperatures between these exemplary temperatures.

Various methods can be utilized to drive the cells through the constrictions. For example, pressure can be applied by a pump on the entrance side (e.g., gas cylinder, or compressor), a vacuum can be applied by a vacuum pump on the exit side, capillary action can be applied through a tube, and/or the system can be gravity fed. Displacement based flow systems can also be used (e.g., syringe pump, peristaltic pump, manual syringe or pipette, pistons, etc.). In some aspects, the cells are passed through the constrictions by positive pressure. In some aspects, the cells are passed through the constrictions by constant pressure or variable pressure. In some aspects, pressure is applied using a syringe. In some aspects, pressure is applied using a pump. In some aspects, the pump is a peristaltic pump or a diaphragm pump. In some aspects, pressure is applied using a vacuum. In some aspects, the cells are passed through the constrictions by g-force. In some aspects, the cells are passed through the constrictions by capillary pressure.

In some aspects, fluid flow directs the cells through the constrictions. In some aspects, the fluid flow is turbulent flow prior to the cells passing through the constriction. Turbulent flow is a fluid flow in which the velocity at a given point varies erratically in magnitude and direction. In some aspects, the fluid flow through the constriction is laminar flow. Laminar flow involves uninterrupted flow in a fluid near a solid boundary in which the direction of flow at every point remains constant. In some aspects, the fluid flow is turbulent flow after the cells pass through the constriction. The velocity at which the cells pass through the constrictions can be varied. In some aspects, the cells pass through the constrictions at a uniform cell speed. In some aspects, the cells pass through the constrictions at a fluctuating cell speed.

In some aspects, a combination treatment is used to deliver a payload, e.g., the methods described herein followed by exposure to an electric field downstream of the constriction. In some aspects, the cell is passed through an electric field generated by at least one electrode after passing through the constriction. In some aspects, the electric field assists in delivery of a payload to a second location inside the cell such as the cell nucleus. In some aspects, one or more electrodes are in proximity to the cell-deforming constriction to generate an electric field. In some aspects, the electric field is between about 0.1 kV/m to about 100 MV/m. In some aspects, an integrated circuit is used to provide an electrical signal to drive the electrodes. In some aspects, the cells are exposed to the electric field for a pulse width of between about 1 ns to about 1 s and a period of between about 100 ns to about 10 s.

### Induction of Immune Response

As is apparent from the present disclosure, polynucleotides described herein (e.g., comprising multiple coding regions that are linked) can be useful in inducing an immune response in a subject in need thereof. For instance, as demonstrated herein, because the polynucleotides described herein comprise multiple coding regions that are linked, administering a polynucleotide of the present disclosure to a subject can result in the induction of an immune response to one or more of the antigens encoded by the multiple coding regions. Accordingly, in some aspects, provided herein is a method for inducing a multi-antigen-specific immune response in a subject in need thereof, comprising administering to the subject a polynucleotide, comprising multiple coding regions that are linked, and wherein the multiple coding regions encode for the multi-antigens. In some aspects, the method comprises administering any of the polynucleotides of the present disclosure. In some aspects, the method comprises administering to the subject a polynucleotide, which comprises at least a first coding region encoding a first antigen and a second coding region encoding a second antigen, wherein the first coding region and the second coding region are linked, and wherein after the administration, an immune response against both the first antigen and the second antigen are induced in the subject. In some aspects, the method can comprise administering to the subject a cell that has been modified to comprise the polynucleotide, such that the cell expresses the multiple antigens.

In some aspects, after the administration, the immune response against the first antigen is increased as compared to the immune response in a reference subject. In some aspects, the reference subject comprises the subject prior to the administration. In some aspects, the reference subject comprises a corresponding subject that did not receive an administration of the polynucleotide (i.e., comprising at least a first coding region encoding a first antigen and a second coding region encoding a second antigen, wherein the first coding region and the second coding region are linked). In some aspects, the reference subject comprises a corresponding subject that received an administration of at least two separate polynucleotides, wherein the first antigen and the second antigen were encoded on separate polynucleotides. In some aspects, as compared to the reference subject, the immune response against the first antigen in the subject after the administration is increased by at least about 10%, at least about 20%, at least about 30%, at least about 40%, at least about 50%, at least about 60%, at least about 70%, at least about 80%, at least about 90%, or at least about 100%. In some aspects, as compared to the reference subject, the immune response against the first antigen in the subject after the administration is increased by at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 35-fold, at least about 40-fold, at least about 45-fold, or at last about 50-fold or more.

In some aspects, after the administration, the immune response against the second antigen is increased as compared to the immune response in the reference subject. In some aspects, as compared to the reference subject, the immune response against the second antigen in the subject after the administration is increased by at least about 10%, at least about 20%, at least about 30%, at least about 40%, at least about 50%, at least about 60%, at least about 70%, at least about 80%, at least about 90%, or at least about 100%. In some aspects, as compared to the reference subject, the immune response against the second antigen in the subject after the administration is increased by at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 35-fold, at least about 40-fold, at least about 45-fold, or at last about 50-fold or more.

In some aspects, after the administration, the immune response against both the first antigen and the second antigen is increased as compared to the immune response in the reference subject. In some aspects, as compared to the reference subject, the immune response against both the first antigen and the second antigen in the subject after the administration is increased by at least about 10%, at least about 20%, at least about 30%, at least about 40%, at least about 50%, at least about 60%, at least about 70%, at least about 80%, at least about 90%, or at least about 100%. In some aspects, as compared to the reference subject, the immune response against the first antigen and the second antigen in the subject after the administration is increased by at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 35-fold, at least about 40-fold, at least about 45-fold, or at last about 50-fold or more.

Accordingly, in some aspects, provided herein is a method of inducing an enhanced immune response in a subject in need thereof. In some aspects, such a method comprises administering to the subject a polynucleotide comprising multiple coding regions, wherein the multiple coding regions are linked, and wherein the multiple coding regions encode for multiple antigens. In some aspects, the method can comprise administering to the subject a cell that has been modified to comprise the polynucleotide, such that the cell expresses the multiple antigens.

In some aspects, an enhanced immune response comprises: (i) an increase in the magnitude of the induced immune response as compared to a reference immune response, (ii) an increase in the breadth of the induced immune response as compared to a reference immune response, (iii) an increase in the duration of the induced immune response as compared to a reference immune response, or (iv) any combination of (i) to (iii). As described herein, in some aspects, the reference subject comprises the subject prior to the administration. In some aspects, the reference subject comprises a corresponding subject that did not receive an administration of the polynucleotide (i.e., comprising multiple coding regions that are linked and encode multiple antigens). In some aspects, the reference subject comprises a corresponding subject that received an administration of multiple polynucleotides, wherein each of the multiple antigens are encoded on separate polynucleotides.

In some aspects, as compared to the reference subject, the magnitude of the induced immune response is increased by at least about 10%, at least about 20%, at least about 30%, at least about 40%, at least about 50%, at least about 60%, at least about 70%, at least about 80%, at least about 90%, or at least about 100%. In some aspects, as compared to the reference subject, the magnitude of the induced immune response is increased by at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 35-fold, at least about 40-fold, at least about 45-fold, or at last about 50-fold or more.

In some aspects, as compared to the reference subject, the breadth of the induced immune response is increased by at least about 10%, at least about 20%, at least about 30%, at least about 40%, at least about 50%, at least about 60%, at least about 70%, at least about 80%, at least about 90%, or at least about 100%. In some aspects, as compared to the reference subject, the breadth of the induced immune response is increased by at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 35-fold, at least about 40-fold, at least about 45-fold, or at last about 50-fold or more.

In some aspects, as compared to the reference subject, the duration of the induced immune response is increased by at least about 10%, at least about 20%, at least about 30%, at least about 40%, at least about 50%, at least about 60%, at least about 70%, at least about 80%, at least about 90%, or at least about 100%. In some aspects, as compared to the reference subject, the duration of the induced immune response is increased by at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 35-fold, at least about 40-fold, at least about 45-fold, or at last about 50-fold or more.

In some aspects, as compared to the reference subject, both the magnitude and the breadth of the induced immune response is increased in the subject. In some aspects, as compared to the reference subject, both the magnitude and duration of the induced immune response is increased in the subject. In some aspects, as compared to the reference subject, both the breadth and duration of the induced immune response is increased in the subject. In some aspects, as compared to the reference subject, the magnitude, the breadth, and the duration of the induced immune response is increased in the subject.

As will be apparent from the present disclosure, the enhanced immune response induced by the polynucleotides described herein or by the cells modified to comprise the polynucleotides can be useful in treating a wide range of diseases or conditions. In some aspects, provided herein is a method of treating a disease or condition in a subject in need thereof, comprising administering to the subject any of the polynucleotides of the present disclosure. In some aspects of the present disclosure relates to a method of treating a disease or condition in a subject in need thereof, comprising administering to the subject a polynucleotide comprising at least a first coding region encoding a first antigen and a second coding region encoding a second antigen, wherein the first coding region and the second coding region are linked, wherein the first antigen and the second antigen are associated with the disease or condition. Accordingly, it will be apparent to those skilled in the arts that the polynucleotides described herein (or cells modified to comprise the polynucleotides) can be used to treat any diseases or conditions, which are associated with a particular antigen. Not to be bound by any one theory, by encoding multiple antigens associated with a disease or conditions within the multiple coding regions of a polynucleotide described herein, an immune response against the multiple antigens can be induced. In some aspects, such an immune response can be useful in treating the disease or condition. For instance, in some aspects, a polynucleotide described herein comprises multiple coding regions that are linked, wherein the multiple coding regions encode multiple KRAS mutant antigens (e.g., comprising the G12D and/or G12V amino acid substitution). Such a polynucleotide can be used to treat cancers that are associated with the KRAS mutant expression.

The following examples are offered by way of illustration and not by way of limitation.

### EXAMPLES

### Example 1: Analysis of Antigen Expression with 5 Linked Antigens

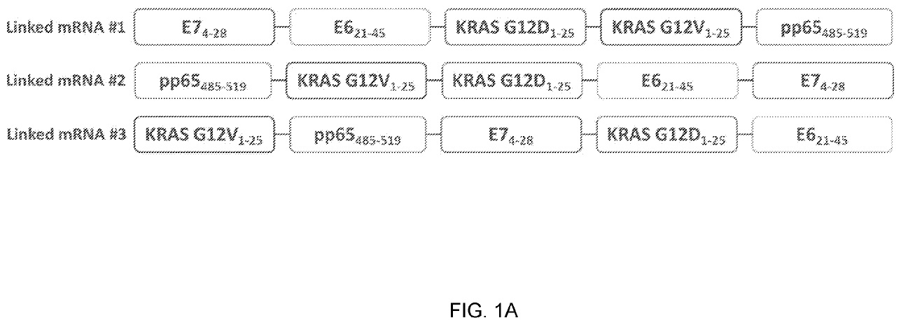

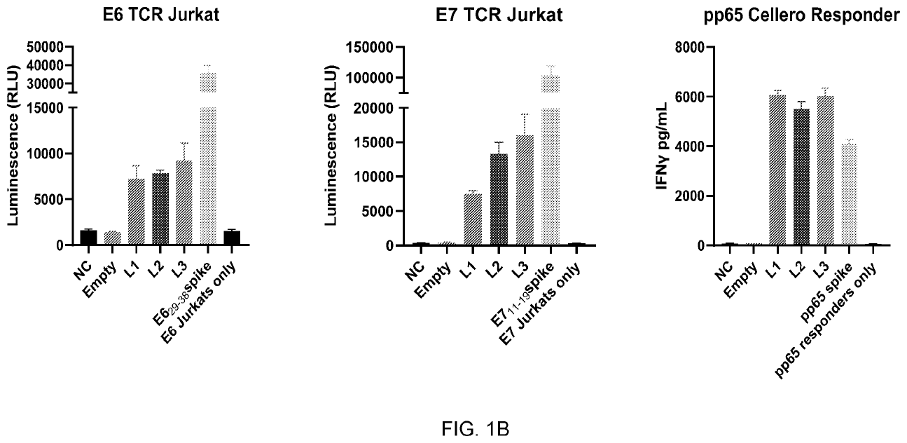

In order to determine if immune cells squeezed with a single mRNA encoding for 5 antigen fragments linked together can elicit multiple antigen-specific immune responses simultaneously, human PBMCs were squeezed with three different linked antigen mRNA constructs each encoding about 25 amino acid long fragments of HPV16 E6, HPV16 E7, CMV pp65, KRAS_{G12V}, and KRAS_{G12D} antigens. See FIG. 1A. The 5 antigen fragments in each mRNA construct were linked in an order different from the other two mRNA constructs. The presentation of immunogenic epitopes from these encoded antigens on MHC class I cells was evaluated using antigen-specific responder cells.

E6 T-cell receptor (TCR), E7 TCR, and KRAS_{G12V} TCR Jurkat-Lucia NFAT reporter cells were generated by transducing either a KRAS_{G12V}-specific TCR, E6₂₉₋₃₈-specific TCR, or E7₁₁₋₁₉-specific TCR expressing lentivirus into Jurkat-Lucia NFAT cells (InvivoGen) that have had endogenous TCRα/β knocked out. Engagement of these three TCRs with the corresponding peptide-MHC (pMHC) complex results in activation of the NFAT signaling pathway and secretion of Lucia luciferase.

PBMCs from a human HLA-A*02+ HLA-A*11+ donor were prepared at a density of 5×10⁷ cells/mL, and squeezed through a constriction of 3.5 μm width, 10 μm length, and 70 μm depth at 60 psi, with 250 μg/mL of an mRNA construct encoding 5 antigen fragments linked together (5×L1, 5×L2, or 5×L3) or with no cargo (empty squeeze), in RPMI 1640 medium at room temperature. The squeeze-loaded PMBCs were transferred to RPMI+10% fetal bovine serum+1× Pen/Strep (R10) to quench at room temperature. The squeeze-loaded PBMCs were washed twice in co-culture medium (R10) before resuspension in fresh co-culture medium.

2.5×10⁵ squeeze-loaded PBMCs were then placed in co-culture with 1.25×10⁵ E6 TCR Jurkat cells or 5×10⁴ E7 TCR Jurkat cells in a 96-well plate. 5×10⁵ squeeze-loaded PBMCs were then placed in co-culture with 2.5×10⁵ KRAS_{G12V} TCR Jurkat cells in a 96-well plate. As a positive control, 1 μM of E6₂₉₋₃₈, E7₁₁₋₁₉, or KRAS_{G12V} (7-16) minimal epitope was added directly to co-cultures of untreated PBMCs and the associated Jurkat responder cells. After incubating the co-culture for 16 to 18 hours at 37° C., co-culture supernatants were harvested. The levels of secreted Lucia luciferase in the culture supernatant were measured via luminescent after addition of a coelenterazine substrate.

For measuring responses to the pp65 fragment in the linked antigen mRNA, 2.5×10⁵ squeeze-loaded PBMCs were then placed in co-culture with 6.25×10⁴ CMV pp65-specific T cells (Cellero) in a 96-well plate. As a positive control, 1 μM of pp65₄₉₅₋₅₀₃ minimal epitope was added directly to co-cultures of untreated PBMCs and CMV pp65-specific T cells. After incubating the co-culture for 16 to 18 hours at 37° C., co-culture supernatants were harvested. To assess the activation of the pp65-specific T cells, IFNγ concentrations in the co-culture supernatants were measured using the Ella immunoassay instrument (Bio-Techne) according to the manufacturer's protocol.

As shown in FIG. 1B, human PBMCs squeeze-loaded with a linked antigen mRNA and co-cultured with either CMV pp65-specific T cells, KRAS_{G12V} TCR Jurkat cells, E6 TCR Jurkat cells, or E7 TCR Jurkat cells led to an increase in activation of the associated responder cells compared to the empty squeeze control. These results demonstrate that human PBMCs squeeze-loaded with linked antigen mRNA can elicit immune responses to multiple antigen fragments simultaneously.

### Example 2: Analysis of Antigen Expression with 10 Linked Antigens

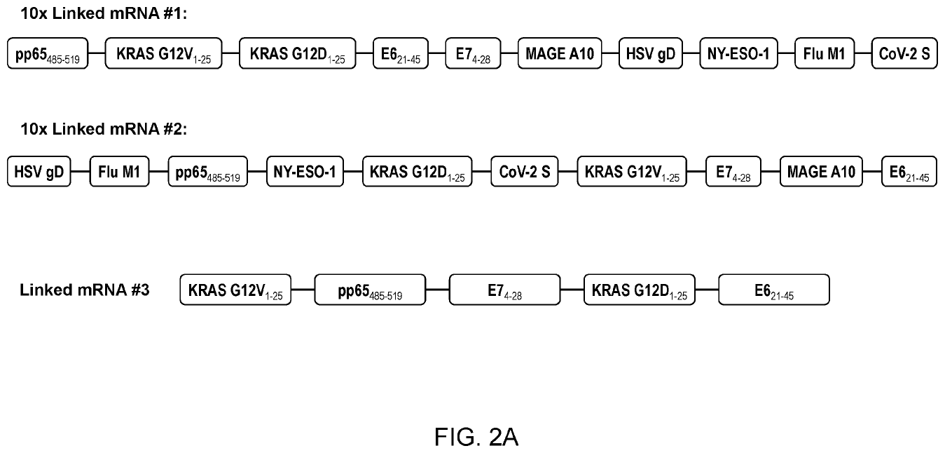

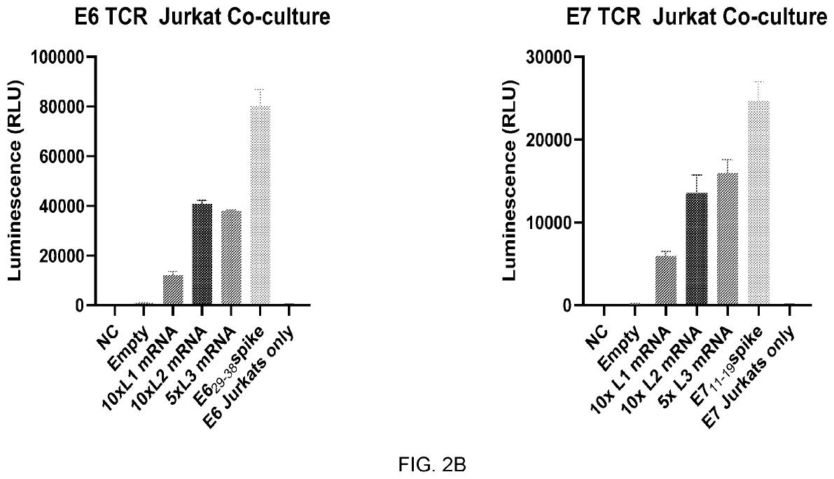

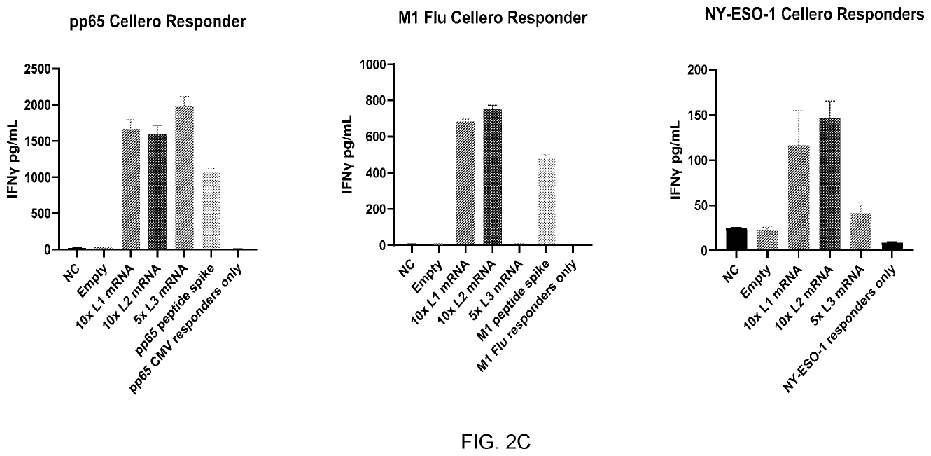

In order to determine if immune cells squeezed with a single mRNA encoding up to 10 antigen fragments linked together can elicit multiple antigen-specific immune responses simultaneously, human PBMCs were squeezed with two different linked antigen mRNA constructs each encoding about 25 amino acid long fragments of HPV16 E6, HPV16 E7, CMV pp65, KRAS_{G12V}, KRAS_{G12D}, Flu M1, NY-ESO-1, HSV gD, SARS-COV2 S, and MAGE-A10 antigens. The 10 antigen fragments in each mRNA construct were linked in an order that is different from the other. See FIG. 2A. The presentation of immunogenic epitopes from these encoded antigens on MHC class I cells was evaluated using antigen-specific responder cells.

E6 TCR and E7 TCR Jurkat-Lucia NFAT reporter cells were generated by transducing either an E6₂₉₋₃₈-specific TCR or E7₁₁₋₁₉-specific TCR expressing lentivirus into Jurkat-Lucia NFAT cells (InvivoGen) that have had endogenous TCRα/β knocked out. Engagement of these TCRs with the corresponding peptide-MHC (pMHC) complex results in activation of the NFAT signaling pathway and secretion of Lucia luciferase.

PBMCs from a human HLA-A*02 HLA-A*11′ donor were prepared at a density of 5×10⁷ cells/mL, and squeezed through a constriction of 3.5 μm width, 10 μm length, and 70 μm depth at 60 psi, with 250 μg/mL of an mRNA construct encoding 10 antigen fragments linked together (10×L1 or 10×L2), with 250 g/mL of an mRNA construct encoding 5 antigen fragments linked together (5×L3), or with no cargo (empty squeeze), in RPMI 1640 medium at room temperature. The squeeze-loaded PMBCs were transferred to RPMI+10% fetal bovine serum+1× Pen/Strep (R10) and quenched at room temperature. The squeeze-loaded PBMCs were washed twice in co-culture medium (R10) before resuspension in fresh co-culture medium.

2.5×10⁵ squeeze-loaded PBMCs were then placed in co-culture with 1.25×10⁵ E6 TCR Jurkat cells or 5×10⁴ E7 TCR Jurkat cells in a 96-well plate. As a positive control, 1 μM of E6₂₉₋₃₈ or E7₁₁₋₁₉ minimal epitope was added directly to co-cultures of untreated PBMCs and the associated Jurkat responder cells. After incubating the co-culture for 16 to 18 hours at 37° C., co-culture supernatants were harvested. The levels of secreted Lucia luciferase in the culture supernatant were measured via luminescent after addition of a coelenterazine substrate.

2.5×10⁵ squeeze-loaded PBMCs were placed in co-culture with 6.25×10⁴ CMV pp65, M1 Flu, NY-ESO-1, or MAGE-A10 specific T cells (Cellero) in 96-well plates. As a positive control, 1 μM of M1₅₈₋₆₆ or pp65₄₉₅₋₅₀₃ minimal epitope was added directly to co-cultures of untreated PBMCs and the associated antigen-specific T cells. After incubating the co-culture for 16 to 18 hours at 37° C., co-culture supernatants were harvested. To assess the activation of the antigen-specific T cells, IFNγ concentrations in the co-culture supernatants were measured using the Ella immunoassay instrument (Bio-Techne) according to the manufacturer's protocol.

As shown in FIGS. 2B and 2C, human PBMCs squeeze-loaded with mRNA constructs encoding 10 antigen fragments linked together and co-cultured with either CMV pp65-specific T cells, Flu M1-specific T cells, NY-ESO-1-specific T cells, E6 TCR Jurkat cells, or E7 TCR Jurkat cells led to an increase in activation of the associated responder cells compared to the empty squeeze control. These results demonstrate that human PBMCs squeeze-loaded with linked antigen mRNA can elicit immune responses to multiple antigen fragments simultaneously.

### Example 3: In Vitro Translation Efficiency of mRNA Comprising Linked Antigens

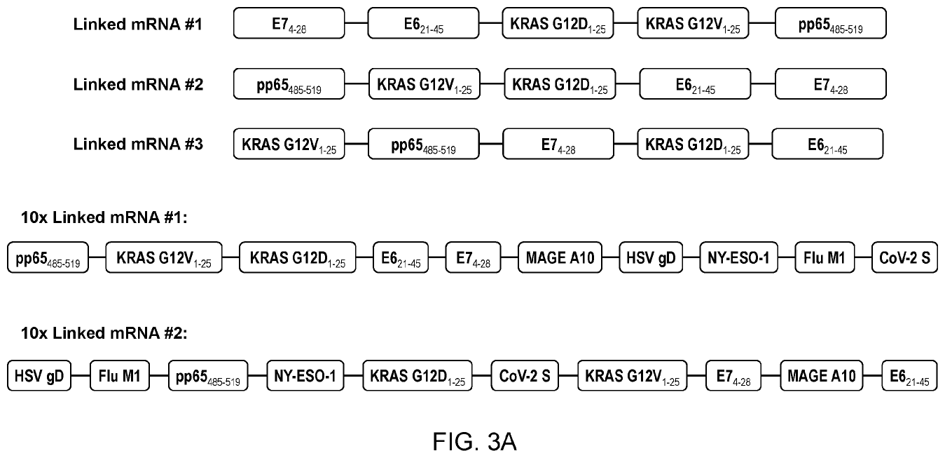

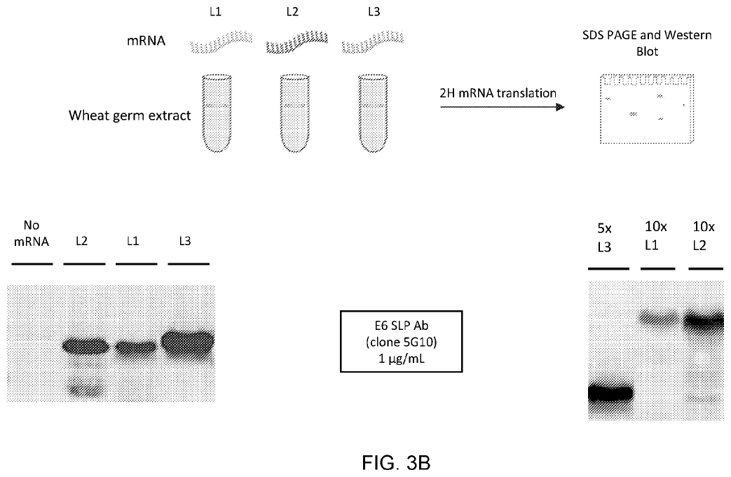

To evaluate in vitro translation efficiency of mRNA constructs encoding for 5 antigen fragments linked together (HPV16 E6, HPV16 E7, CMV pp65, KRAS_{G12V}, and KRAS_{G12D}) or encoding for 10 antigen fragments linked together (HPV16 E6, HPV16 E7, CMV pp65, KRAS_{G12V}, KRAS_{G12D}, Flu M1, NY-ESO-1, HSV gD, SARS-COV2 S, and MAGE-A10), each linked antigen mRNA construct was individually mixed with wheat germ extract, a cell-free expression system. See FIG. 3A.

TnT® SP6 High-Yield Wheat Germ Protein Master Mix (Promega) was thawed and immediately placed on ice. Each linked antigen mRNA construct was diluted with diH₂O to a final concentration of 1 μg/μL. For each linked antigen mRNA construct, 2 μg of mRNA was added to 30 μL of wheat germ extract and brought up to a total volume of 50 μL with diH₂O, and the reaction was incubated at 25° C. for 2 hours. After the 2-hour incubation, the samples were placed on ice to terminate the reaction, and 50 μL of the translated sample was mixed with 25 μL of sample buffer.

For the SDS-PAGE gel, samples were heated for 2 minutes at 95° C. before being loaded on a 4 to 12% protein gel (Invitrogen). The samples were run on the gel at 100-130V for about 90 minutes until the dye reached the bottom of the gel. The gel was transferred onto a nitrocellulose membrane and blotted for linked antigens using a primary antibody generated to HPV E6₂₉₋₃₈ SLP (clone 5G10) and Goat anti-rabbit secondary antibody. Translation was assessed by presence of protein bands around 26 kDa for mRNA constructs encoding for 5 linked antigen fragments, and around 35 kDa for mRNA constructs encoding for 10 linked antigen fragments.

As shown in FIG. 3B, translation of each linked antigen mRNA construct was detected at various magnitudes. Of the three mRNA constructs each encoding 5 model antigen fragments linked together, construct 5×L3 was the most heavily translated, followed by 5×L2 and lastly 5×L1. Of the two mRNA constructs each encoding 10 model antigen fragments linked together, 10×L2 was more heavily translated than 10×L1.

### Example 4: Induction of KRAS Mutant-Specific Immune Response

In order to determine if immune cells squeezed with a single mRNA encoding for 5 antigen fragments linked together can elicit KRAS_{G12V} and KRAS_{G12D} antigen-specific immune responses simultaneously, human PBMCs were squeezed with three different linked antigen mRNA constructs each encoding about 25 amino acid long fragments of HPV16 E6, HPV16 E7, CMV pp65, KRAS_{G12V}, and KRAS_{G12D} antigens. The 5 antigen fragments in each mRNA construct are linked in an order different from the other two mRNA constructs. The presentation of immunogenic epitopes from these encoded antigens on MHC class I cells was evaluated using antigen-specific responder cells.

KRAS_{G12D} TCR and KRAS_{G12V} TCR Jurkat-Lucia NFAT reporter cells were generated by transducing either a KRAS_{G12V}-specific TCR or KRAS_{G12D}-specific TCR expressing lentivirus into Jurkat-Lucia NFAT cells (InvivoGen) that have had endogenous TCRα/β knocked out. Engagement of these TCRs with the corresponding peptide-MHC (pMHC) complex results in activation of the NFAT signaling pathway and secretion of Lucia luciferase.

PBMCs from a human HLA-A*02-HLA-A*11+ donor were prepared at a density of 5×10⁷ cells/mL, and squeezed through a constriction of 3.5 μm width, 10 μm length, and 70 μm depth at 60 psi, with 250 μg/mL of an mRNA construct encoding 5 antigen fragments linked together (5×L1, 5×L2, or 5×L3) or with no cargo (empty squeeze), in RPMI 1640 medium at room temperature. The squeeze-loaded PMBCs were transferred to RPMI+10% fetal bovine serum+1× Pen/Strep (R10) to quench at room temperature. The squeeze-loaded PBMCs were washed twice in co-culture medium (R10) before resuspension in fresh co-culture medium.

5×10⁵ squeeze-loaded PBMCs were then placed in co-culture with 2.5×10⁵ KRAS_{G12V} TCR Jurkat cells or KRAS_{G12D} TCR Jurkat cells in a 96-well plate. As a positive control, 1 μM of KRAS_{G12V} (7-16) or KRAS_{G12D} (7-16) minimal epitope was added directly to co-cultures of untreated PBMCs and the associated Jurkat responder cells. After incubating the co-culture for 16 to 18 hours at 37° C., co-culture supernatants were harvested. The levels of secreted Lucia luciferase in the co-culture supernatant were measured via luminescent after addition of a coelenterazine substrate.

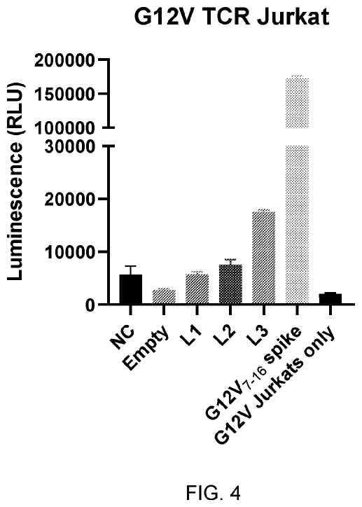

As shown in FIG. 4, human PBMCs squeeze-loaded with linked antigen mRNA and co-cultured with KRAS_{G12V} TCR Jurkat cells led to an increase in activation of the associated responder cells compared to the empty squeeze control. These results demonstrate that human PBMCs squeeze-loaded with linked antigen mRNA can elicit immune responses to KRAS mutant antigens.

### Example 5: Effect of Signal 2/3 on Immune Response Induction by mRNA Comprising Linked Antigens

In order to measure functional CD8+ T cell responses of immune cells squeezed with a single mRNA encoding for 5 antigen fragments linked together with or without additional Signal 2/3 mRNAs (encoding CD86, mbIL-2, and mbIL-12), human PBMCs were squeezed with an mRNA construct encoding about 25 amino acid long aa fragments of HPV16 E6, HPV16 E7, CMV pp65, KRAS_{G12V}, and KRAS_{G12D} antigens, with or without Signal 2/3 mRNAs, or squeezed with Signal 2/3 mRNAs only. Functional CD8+ T cell responses from these antigens, with or without Signal 2/3, was evaluated using antigen-specific primary cells.

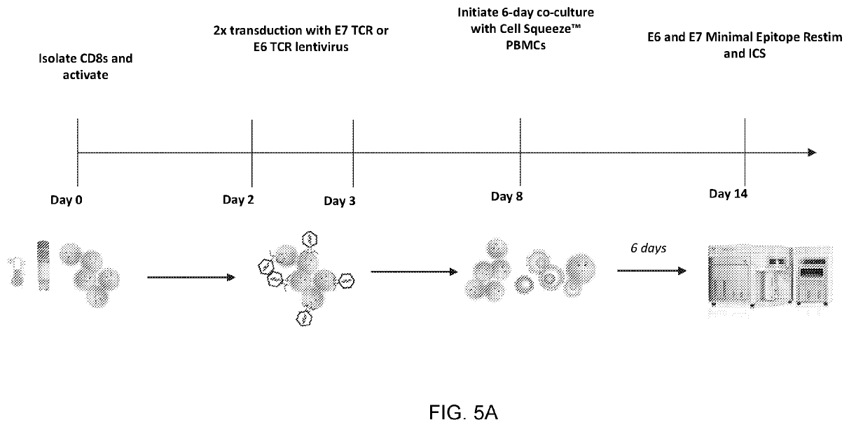

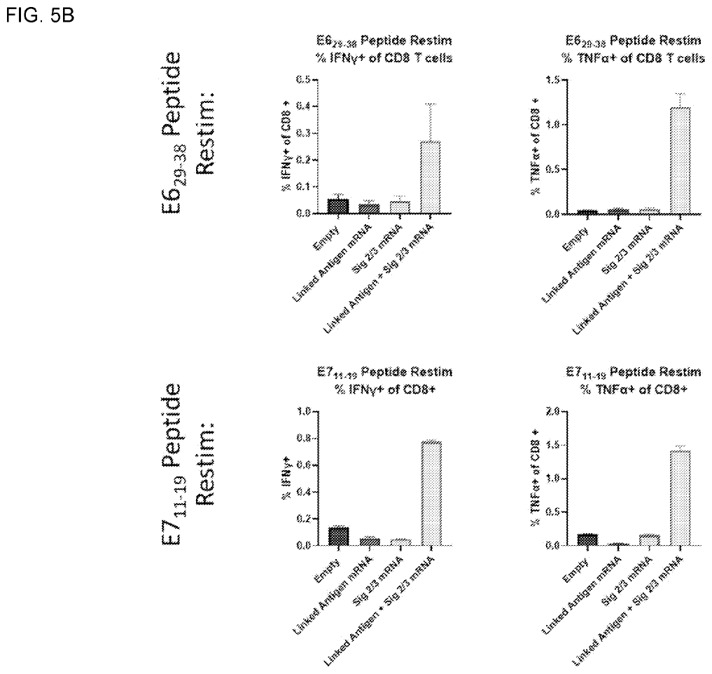

As illustrated in FIG. 5A, CD8+ T cells were isolated from a HLA-A*02+ donor and activated with CD3/CD28 Dynabeads and rhIL-2 for 2 days. The CD8+ T cells were transduced on 2 consecutive days with either an E6_{29,38}-specific TCR or E7₁₁₋₁₉-specific TCR expressing lentivirus. Residual lentivirus was removed and cells were expanded for 5 days before co-cultures. Engagement of these TCRs with the corresponding peptide-MHC (pMHC) complex results in production of IFNγ.

PBMCs from the same HLA-A*02+ donor were prepared at a density of 5×10⁷ cells/mL, and squeezed through a constriction of 3.5 μm width, 10 μm length, and 70 μm depth at 60 psi, with 250 μg/mL of a linked antigen mRNA (L2), the linked antigen mRNA and Signal 2/3 mRNAs, the Signal 2/3 mRNAs alone, or with no cargo (empty squeeze), in RPMI 1640 medium at room temperature. The squeeze-loaded PMBCs were transferred to RPMI+10% fetal bovine serum+1× Pen/Strep (R10) to quench at room temperature. The squeeze-loaded PBMCs were washed twice in co-culture medium (R10) before resuspension in fresh co-culture medium.

5×10⁴ of squeeze-loaded PBMCs were then placed in co-culture with 0.2% E6 TCR or E7 TCR Tetramer+CD8+ T cells and 1.28×10⁵ untreated PBMCs in a 96-well plate. After incubating the co-culture for 6 days at 37° C., co-cultured cells were restimulated with the corresponding E6₂₉₋₃₈ or E7₁₁₋₁₉ peptide and golgiblock, or with golgiblock alone as a negative control, and incubated at 37° C. After 6 hours of restimulation, the cells were intracellularly stained, and expression of IFNγ and TNFα was assessed via flow cytometry. The frequency of E7₁₁₋₂₉ Pentamer+ or E6₂₉₋₃₈ Tetramer+CD8+ T cells after the 6 day co-culture was also assessed via flow cytometry.

As shown in FIG. 5B, human PBMCs squeeze-loaded with linked antigen mRNA and Signal 2/3 mRNAs, and then co-cultured with E6 TCR-transduced CD8+ T cells or E7 TCR-transduced CD8-T cells, led to an increase in activation of antigen-specific CD8+ T cells compared to the human PBMCs squeezed with only Signal 2/3 mRNAs. These results demonstrate that human PBMCs squeeze-loaded with linked antigen mRNA can elicit strong immune responses when combined with Signal 2/3 mRNAs.

### Example 6: Analysis of KRAS G12V and G12D Specific Responses after Squeeze Processing with mRNA Encoding Two KRAS Mutant Linked Antigens

To further assess the functional capability of the polynucleotides described herein (e.g., comprising multiple coding regions that are linked), human PBMCs were squeeze processed with one of the linked mRNAs described below.
(1) linked mRNA construct encoding: (a) ˜25 aa fragments (overlapping with the 1-25 aa sequence in the whole KRAS protein and mutations on the 12^{th} codon) KRAS_{G12D} and KRAS_{G12V} antigens, (b) CD86 (i.e., signal 2), (c) membrane-bound IL-2 (i.e., signal 3), and (d) membrane-bound IL-12 (i.e., signal 3) (“Linked Antigen+2/3”);
(2) linked mRNA construct encoding only the ˜25 aa fragments (overlapping with the 1-25 aa sequence in the whole KRAS protein and mutations on the 12^{th} codon) KRAS_{G12D} and KRAS_{G12V} antigens (“G12D-G12V Linked Antigen”);
(3) linked mRNA construct encoding only CD86 (i.e., signal 2), (c) membrane-bound IL-2 (i.e., signal 3), and (d) membrane-bound IL-12 (i.e., signal 3) (“signal 2/3”);
(4) linked mRNA construct encoding: (a) single ˜25 aa fragment comprising the G12V mutation, (b) CD86 (i.e., signal 2), (c) membrane-bound IL-2 (i.e., signal 3), and (d) membrane-bound IL-12 (i.e., signal 3) (“G12V+2/3”);
(5) linked mRNA construct encoding: (a) single ˜25 aa fragment comprising the G12D mutation, (b) CD86 (i.e., signal 2), (c) membrane-bound IL-2 (i.e., signal 3), and (d) membrane-bound IL-12 (i.e., signal 3) (“G12D+2/3”);
(6) linked mRNA construct encoding: (a) ˜25 aa fragment overlapping with the first 1-25 aa of native wild-type KRAS; (b) CD86 (i.e., signal 2), (c) membrane-bound IL-2 (i.e., signal 3), and (d) membrane-bound IL-12 (i.e., signal 3) (“WT+2/3”);
(7) mRNA construct encoding only single ˜25 aa fragment comprising the G12V mutation (i.e., no signals 2 and 3) (“G12V”);
(8) mRNA construct encoding only single ˜25 aa fragment comprising the G12D mutation (i.e., no signals 2 and 3) (“G12D”); and
(9) mRNA construct encoding only ˜25 aa fragment overlapping with the first 1-25 aa of native wild-type KRAS (i.e., no signals 2 and 3) (“Wild Type”).

### Methods

### Generation of Responder Cells:

To generate the A*11-restricted G12V and G12D TCR Jurkat responder cells, Jurkat-Lucia NFAT TCR α/β KO cells, custom manufactured at Creative Biolabs, were transduced with A*11-restricted G12V₇₋₁₆ and G12D₇₋₁₆-specific TCR expressing lentivirus. Residual virus was removed, and the cells were then subjected to puromycin selection and cultured for a few days prior to being subjected to mouse TCR (mTCR) enrichment to increase the frequency of mTCR+ cells. Following mTCR enrichment, the cells were cultured for a few days, characterized by flow and frozen down for use in co-culture assays. The resulting A*11-restricted G12V TCR Jurkat and G12D TCR Jurkat responder cells expressed an HLA-A*11-restricted G12V7-16-specific TCR or an HLA-A*11-restricted G12D7-16-specific TCR, respectively, on the cell surface. Engagement of these TCRs with the corresponding peptide-MHC (pMHC) complex, provided in the form of PBMCs squeezed with KRAS mutant mRNAs or incubated with G12V7-16 or G12D7-16 A*11-restricted peptides, resulted in activation of the NFAT signaling pathway and secretion of Lucia luciferase. The levels of secreted Lucia luciferase in the culture supernatant were measured using a coelenterazine substrate. The resulting luminescent signal correlated to the amount of secreted Lucia luciferase, confirming that the cells were properly produced.

### Squeeze Processing:

PBMCs from an HLA-A*11+ donor were prepared at a density of 200×10⁶ cells/mL, and allowed to rest at 4-8° C. for ˜15 minutes. Then, the PBMCs were squeeze processed with a constriction of 3.5 μm width, 10 μm length, and 70 μm depth at 60 psi with 250 μg/mL of one of the mRNA constructs described above in RPMI 1640 medium at room temperature (mRNA solutions were cooled at 4-8° C. for ˜15 minutes prior to mixing with cells and squeezing). PBMCs squeeze processed with no mRNAs (i.e., empty squeezing) were used as one of the controls. Following the squeeze processing, the squeeze processed PBMCs were transferred to R10 media (RPMI+10% fetal bovine serum (FBS)+1× Pen/Strep) to quench at room temperature, subsequently washed in R10 and cryopreserved at 5×10⁶ cells/mL and stored in liquid nitrogen for future use.

### Assay:

On the day of co-culture set-up, the HLA-A*11-restricted G12V and G12D TCR Jurkats were prepared at 2.5×10⁶ cells/mL in IMDM+10% FBS. Cryopreserved squeeze processed or unprocessed PBMCs were thawed, media exchanged to IMDM+10% FBS at a density of 5×10⁶ cells/mL. G12V or G12D TCR Jurkats were co-cultured with PBMCs at 1:2 ratio (250,000 Jurkat cells with 500,000 PBMCs) in a tissue culture treated (TCT) 96-well U-bottom plate overnight (18-24 hours). HLA-A*11-restricted G12V₇₋₁₆ and G12D₇₋₁₆ or WT₇₋₁₆ minimal epitopes were added to co-cultures with Jurkat cells and unprocessed PBMCs to control for antigen mutation-specific Jurkat reactivity. Cell culture supernatants were collected and assayed for the levels of secreted Lucia luciferase via Quanti LUC.

### Results

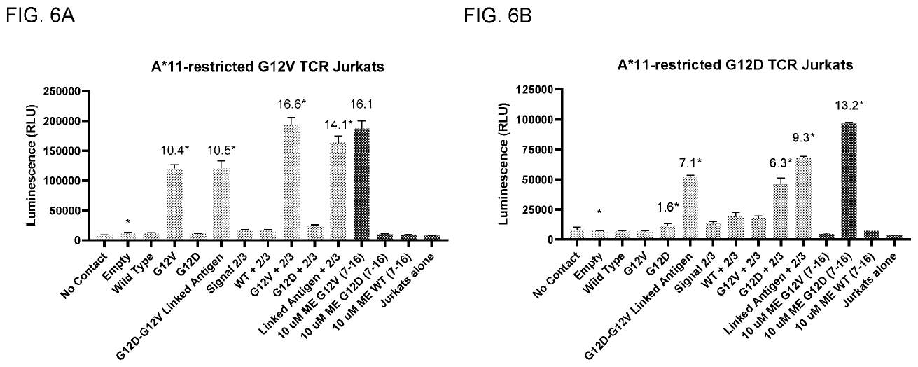

As shown in FIGS. 6A and 6B, human PBMCs squeeze-loaded with KRAS G12D-G12V linked antigen mRNA with or without Signal 2/3 mRNAs and co-cultured with G12V TCR-transduced Jurkat cells or G12D TCR-transduced Jurkat cells led to a KRAS mutant-specific increase in the activation of G12V and G12D TCR-transduced Jurkat cells relative to controls (empty squeeze, Signal 2/3 mRNAs alone, KRAS WT mRNA with or without signal 2/3 mRNAs). PBMCs squeezed with KRAS G12D-G12V linked antigen mRNA elicited similar or stronger mutation specific responses from G12V TCR-transduced or G12D TCR-transduced Jurkat cells than their relevant KRAS mutant single mRNAs, respectively.

These results demonstrate that human PBMCs squeeze-loaded with KRAS mutant linked antigen mRNA can elicit strong mutation specific responses from G12V and G12D TCR-transduced Jurkat cells, similar or stronger than their respective KRAS mutant single antigen mRNA.

### Example 7: Analysis of KRAS G12V and G12D Specific Responses after Squeeze Processing with mRNA Encoding Seven KRAS Mutant Linked Antigens

To assess the functional activity of mRNAs encoding more than two linked antigens, immune cells (PBMCs) were squeeze processed with mRNA constructs comprising seven linked KRAS mutant antigens. The specific mRNA constructs used are provided below. Each of the constructs were used at one of two doses: 250 μg/mL and 500 μg/mL.
(1) linked mRNA construct encoding only ˜25 aa fragments (overlapping with the 1-25 aa sequence in the whole KRAS protein and a mutation on the 12^{th} codon) KRAS_{G12D}, KRAS_{G12V}, KRAS_{G12C}, KRAS_{G13D}, KRAS_{G12A}, KRAS_{G12R} and KRAS_{G12S} antigens (“7 mut_v1”); and
(2) linked mRNA construct encoding only ˜25 aa fragments (overlapping with the 1-25 aa sequence in the whole KRAS protein and a mutation on the 12^{th} codon) KRAS_{G12S}, KRAS_{G12R}, KRAS_{G12A}, KRAS_{G13D}, KRAS_{G12C}, KRAS_{G12V} and KRAS_{G12D} antigens (“7mut_v2”).

### Methods

### Generation of Responder Cells:

To generate A*11-restricted G12V and G12D TCR Jurkat responder cells, Jurkat-Lucia NFAT TCR α/β KO cells, custom manufactured at Creative Biolabs, were transduced with A*11-restricted G12V7-16 and G12D7-16-specific TCR expressing lentivirus (once). Residual virus was removed one-day post TCR transduction, and cells were then subjected to puromycin selection and cultured for a few days prior to being subjected to mouse TCR (mTCR) enrichment to increase frequency of mTCR+ cells. Following mTCR enrichment cells were cultured for a few days, characterized by flow and frozen down for use in co-culture assays. The resulting A*11-restricted G12V TCR Jurkat and G12D TCR Jurkat responder cells express an HLA-A*11-restricted G12V7-16-specific TCR or an HLA-A*11-restricted G12D7-16-specific TCR, respectively, on the cell surface. Engagement of these TCRs with the corresponding peptide-MHC (pMHC) complex, provided in the form of PBMCs squeezed with KRAS mutant mRNAs or incubated with G12V7-16 or G12D7-16 A*11-restricted peptides, results in activation of the NFAT signaling pathway and secretion of Lucia luciferase. The levels of secreted Lucia luciferase in the culture supernatant can be measured using a coelenterazine substrate. The resulting luminescent signal correlates to the amount of secreted Lucia luciferase.

### Squeeze Processing:

PBMCs from an HLA-A*11+ donor were prepared at a density of 200×10⁶ cells/mL, allowed to rest at 4-8° C. for ˜10-15 minutes prior to squeezing through a constriction of 3.5 μm width, 10 μm length, and 70 μm depth at 60 psi with 250 or 500 μg/mL KRAS 7mut_v1 mRNA or 250 or 500 μg/mL KRAS 7mut_v2 mRNA, or with no cargo (empty squeeze) in RPMI 1640 medium at room temperature (mRNA solutions were cooled at 4-8° C. for ˜10-15 minutes prior to mixing with cells and squeezing). Following the squeeze, the squeeze processed PBMCs were transferred to R10 media (RPMI+10% fetal bovine serum (FBS)+1× Pen/Strep) to quench at room temperature, subsequently washed in R10 and cryopreserved at 5-10×10⁶ cells/mL and stored in liquid nitrogen for future use.

### Assay:

On the day of co-culture set-up HLA-A*11-restricted G12V and G12D TCR Jurkats were prepared at 2.5×10⁶ cells/mL in IMDM+10% FBS. Cryopreserved squeezed or unprocessed PBMCs were thawed, media exchanged to IMDM+10% FBS at a density of 5×10⁶ cells/mL. G12V or G12D TCR Jurkats were co-cultured with PBMCs at 1:2 ratio (250,000 Jurkat cells with 500,000 PBMCs) in a tissue culture treated (TCT) 96-well U-bottom plate overnight (18-24 hours). HLA-A*11-restricted G12V₇₋₁₆ and G12D₇₋₁₆ minimal epitopes were added to co-cultures with Jurkat cells and unprocessed PBMCs to control for antigen mutation-specific Jurkat reactivity. Cell culture supernatants were collected and assayed for the levels of secreted Lucia luciferase via Quanti LUC. The resulting luminescence signal correlates with the amount of secreted Lucia luciferase.

### Results:

As shown in 7A and 7B, human PBMCs squeeze-loaded with KRAS seven mutation linked antigen mRNA co-cultured with G12V TCR-transduced or G12D TCR-transduced Jurkat cells led to KRAS mutant-specific responses from TCR-transduced Jurkat cells relative to controls (empty squeeze, unprocessed PBMCs and irrelevant mutation minimal epitope stimulation). G12V specific responses: PBMCs squeezed with KRAS 7mut_v1 mRNA elicited G12V-specific responses from G12V TCR transduced Jurkat cells in a dose dependent manner. PBMCs squeezed with KRAS 7mut_v2 mRNA elicited similar responses at both squeeze concentrations (250, 500 μg/mL) and comparable to responses elicited by PBMCs squeezed with KRAS 7mut_v1 mRNA at 250 μg/mL. G121) specific responses: PBMCs squeezed with KRAS 7mut_v1 mRNA elicited G12D-specific responses from G12D TCR transduced Jurkat cells in a dose dependent manner. Level of G12D responses elicited by PBMCs squeezed with KRAS 7mut_v2 mRNA were similar to responses observed in control groups (untreated PBMCs and empty squeeze).

These results demonstrate that human PBMCs squeeze-loaded with KRAS 7 mutation linked antigen mRNA (KRAS 7mut_v1 construct) can elicit strong mutation specific responses from both G12V and G12D TCR-transduced Jurkat cells.

### Example 8: Further Analysis of KRAS G12V and G12D Specific Responses after Squeeze Processing with mRNA Encoding Seven KRAS Mutant Linked Antigens

To assess the functional activity of mRNAs encoding both the seven KRAS mutant antigens and further encoding CD86 (signal 2), membrane-bound IL-2 (signal 3) and membrane-bould IL-12 (signal 3), immune cells (PBMCs) were squeeze processed with one of the following mRNA constructs:
(1) linked mRNA construct encoding: (a) ˜25 aa fragments (overlapping with the 1-25 aa sequence in the whole KRAS protein and a mutation on the 12^{th} codon) KRAS_{G12D}, KRAS_{G12V}, KRAS_{G12C}, KRAS_{G13D}, KRAS_{G12A}, KRAS_{G12R} and KRAS_{G12S} antigens (termed “KRAS 7mut_v1”), (b) CD86 (i.e., signal 2), (c) membrane-bound IL-2 (i.e., signal 3), and (d) membrane-bound IL-12 (i.e., signal 3) (“7mut_v1+Signal 2/3”);
(2) linked mRNA construct encoding only the ˜25 aa fragments (overlapping with the 1-25 aa sequence in the whole KRAS protein and a mutation on the 12^{th} codon) KRAS_{G12D}, KRAS_{G12V}, KRAS_{G12C}, KRAS_{G13D}, KRAS_{G12A}, KRAS_{G12R} and KRAS_{G12S} antigens (“7mut_v1”);
(3) linked mRNA construct encoding: (a) ˜25 aa fragments (overlapping with the 1-25 aa sequence in the whole KRAS protein and a mutation on the 12^{th} codon) KRAS_{G12S}, KRAS_{G12R}, KRAS_{G12A}, KRAS_{G12D}, KRAS_{G12C}, KRAS_{G12V} and KRAS_{G12D} antigens (termed KRAS 7mut_v2 here), (b) CD86 (i.e., signal 2), (c) membrane-bound IL-2 (i.e., signal 3), and (d) membrane-bound IL-12 (i.e., signal 3) (“7mut_v2+Signal 2/3”);

(4) linked mRNA construct encoding only the ˜25 aa fragments (overlapping with the 1-25 aa sequence in the whole KRAS protein and a mutation on the 12^{th} codon) KRAS_{G12S}, KRAS_{G12R}, KRAS_{G12A}, KRAS_{G13D}, KRAS_{G12C}, KRAS_{G12V} and KRAS_{G12D} antigens (“mut_v2”); and
(5) linked mRNA construct encoding only CD86 (i.e., signal 2), (c) membrane-bound IL-2 (i.e., signal 3), and (d) membrane-bound IL-12 (i.e., signal 3) (“signal 2/3 alone”).

### Methods

### Generation of Responder Cells:

To generate A*11-restricted G12V and G12D TCR Jurkat responder cells, Jurkat-Lucia NFAT TCR α/β KO cells, custom manufactured at Creative Biolabs, were transduced with A*11-restricted G12V₇₋₁₆ and G12D₇₋₁₆-specific TCR expressing lentivirus (once). Residual virus was removed one-day post TCR transduction, and cells were then subjected to puromycin selection and cultured for a few days prior to being subjected to mouse TCR (mTCR) enrichment to increase frequency of mTCR+ cells. Following mTCR enrichment cells were cultured for a few days, characterized by flow and frozen down for use in co-culture assays. The resulting A*11-restricted G12V TCR Jurkat and G12D TCR Jurkat responder cells express an HLA-A*11-restricted G12V₇₋₁₆-specific TCR or an HLA-A*11-restricted G12D₇₋₁₆-specific TCR, respectively, on the cell surface. Engagement of these TCRs with the corresponding peptide-MHC (pMHC) complex, provided in the form of PBMCs squeezed with KRAS mutant mRNAs or incubated with G12V₇₋₁₆ or G12D₇₋₁₆ A*11-restricted peptides, results in activation of the NFAT signaling pathway and secretion of Lucia luciferase. The levels of secreted Lucia luciferase in the culture supernatant can be measured using a coelenterazine substrate. The resulting luminescent signal correlates to the amount of secreted Lucia luciferase.

### Squeeze Processing:

PBMCs from an HLA-A*11+ donor were prepared at a density of 200×10⁶ cells/mL (cell squeeze concentration 100×10⁶ cells/mL), and allowed to rest at 4-8° C. for ˜10-15 minutes. Then, the PBMCs were squeeze processed with a constriction of 3.5 μm width, 10 μm length, and 70 μm depth at 60 psi with 500 μg/mL of one of the mRNA constructs described above in RPMI 1640 medium at room temperature (mRNA solutions were cooled at 4-8° C. for ˜15 minutes prior to mixing with cells and squeezing). Following the squeeze, the squeeze processed PBMCs were transferred to R10 media (RPMI+10% fetal bovine serum (FBS)+1× Pen/Strep) to quench at room temperature, subsequently washed in R10 and cryopreserved at 5×10⁶ cells/mL and stored in liquid nitrogen for future use.

### Assay:

On the day of co-culture set-up HLA-A*11-restricted G12V and G12D TCR Jurkats were prepared at 2.5×10⁶ cells/mL in IMDM+10% FBS. Cryopreserved squeezed or unprocessed PBMCs were thawed, media exchanged to IMDM+10% FBS at a density of 5×10⁶ cells/mL. G12V or G12D TCR Jurkats were co-cultured with PBMCs at 1:2 ratio (250,000 Jurkat cells with 500,000 PBMCs) in a tissue culture treated (TCT) 96-well U-bottom plate overnight (18-24 hours). HLA-A*11-restricted G12V7-16 and G12D7-16 minimal epitopes were added to co-cultures with Jurkat cells and unprocessed PBMCs to control for antigen mutation-specific Jurkat reactivity. Cell culture supernatants were collected and assayed for the levels of secreted Lucia luciferase via Quanti LUC. The resulting luminescence signal correlates with the amount of secreted Lucia luciferase.

### Results

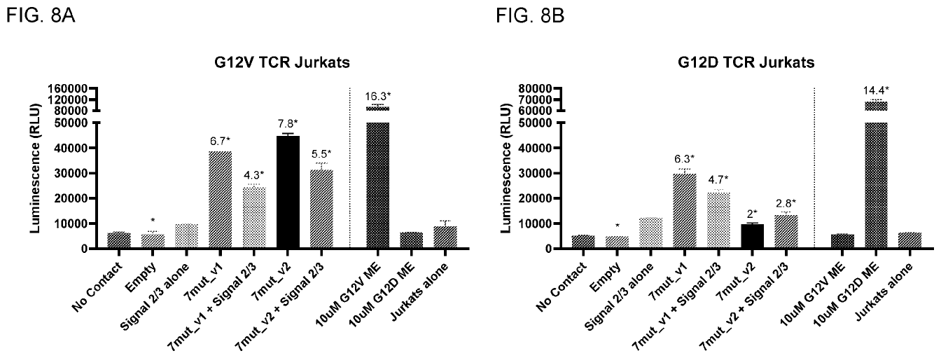

As shown in FIGS. 8A and 8B, human PBMCs squeeze-loaded with KRAS seven mutation linked antigen mRNA with or without Signal 2/3 mRNAs co-cultured with G12V TCR-transduced or G12D TCR-transduced Jurkat cells led to KRAS mutant-specific responses from TCR-transduced Jurkat cells relative to controls (empty squeeze, unprocessed PBMCs, Signal 2/3 mRNAs alone and irrelevant mutation minimal epitope stimulation). G12V specific responses: PBMCs squeezed with KRAS 7mut_v1 mRNA or KRAS 7mut_v2 mRNA at 500 μg/mL (with or without Signal 2/3 mRNAs) elicited G12V-specific responses from G12V TCR transduced Jurkat cells. G12D specific responses: PBMCs squeezed with KRAS 7mut_v1 mRNA at 500 μg/mL (with or without Signal 2/3 mRNAs) elicited G12D-specific responses from G12D TCR transduced Jurkat cells.

These results further demonstrate that human PBMCs squeeze-loaded with KRAS 7 mutation linked antigen mRNA (KRAS 7mut_v1 construct) can elicit strong mutation specific responses from both G12V and G12D TCR-transduced Jurkat cells.

TABLE 2
Exemplary Sequences

| SEQ | | |
|---|---|---|
| ID | | |
| NO | Sequence | Description |
| 1 | MTEYKLVVVGADGVGK | G12D(1-16) |
| 2 | TEYKLVVVGADGVGKSAL | G12D(2-19) |
| 3 | TEYKLVVVGADGVGKSALTIQ | G12D(2-22) |
| 4 | TEYKLVVVGADGVGKSALTIQLIQNHFV | G12D(2-29) |
| 5 | MTEYKLVVVGAVGVGK | G12V(1-16) |
| 6 | TEYKLVVVGAVGVGKSAL | G12V(2-19) |
| 7 | EYKLVVVGAVGVGKS | G12V(3-17) |
| 8 | EYKLVVVGAVGVGKSALTIQLIQNHFVDEYDPTIEDSYRK | G12V(3-42) |
| 9 | VVVGADGVGK | 7-16 G12D |
| 10 | VVVGAVGVGK | 7-16 G12V |
| 11 | VVVGACGVGK | 7-16 G12C |
| 12 | VVGADGVGK | 8-16 G12D |
| 13 | VVGAVGVGK | 8-16 G12V |
| 14 | KLVVVGADGV | 5-14 G12D |
| 15 | GADGVGKSAL | 10-19 G12D |
| 16 | MTEYKLVVVGAGGVGKSALTIQLIQNHFVDEYDPTIEDSYRKQVVIDGETCLLDIL | WT KRAS Isoform 2A |
| | DTAGQEEYSAMRDQYMRTGEGFLCVFAINNTKSFEDIHHYREQIKRVKDSEDVP | (Accession Number: |
| | MVLVGNKCDLPSRTVDTKQAQDLARSYGIPFIETSAKTRORVEDAFYTLVREIRQY | P01116-1) |
| | RLKKISKEEKTPGCVKIKKCIIM | |
| 17 | MTEYKLVVVGAGGVGKSALTIQLIQNHFVDEYDPTIEDSYRKQVVIDGETCLLDIL | WT KRAS Isoform 2B |
| | DTAGQEEYSAMRDQYMRTGEGFLCVFAINNTKSFEDIHHYREQIKRVKDSEDVP | (Accession Number: |
| | MVLVGNKCDLPSRTVDTKQAQDLARSYGIPFIETSAKTRQGVDDAFYTLVREIRK | P01116-2 |
| | HKEKMSKDGKKKKKKSKTKCVIM | |

### INCORPORATION BY REFERENCE

All publications, patents, patent applications and other documents cited in this application are hereby incorporated by reference in their entireties for all purposes to the same extent as if each individual publication, patent, patent application or other document were individually indicated to be incorporated by reference for all purposes.

### EQUIVALENTS

While various specific aspects have been illustrated and described, the above specification is not restrictive. It will be appreciated that various changes can be made without departing from the spirit and scope of the disclosure(s). Many variations will become apparent to those skilled in the art upon review of this specification.

1. An isolated polynucleotide comprising a single ORF with a first nucleotide sequence encoding a first antigen (“first coding region”) and a second nucleotide sequence encoding a second antigen (“second coding region”), wherein the first coding region and the second coding region are linked.

2. The polynucleotide of claim 1, wherein the first coding region and the second coding region are linked by a linker.

3. The polynucleotide of claim 1, wherein:
(a) the first coding region and the second coding region are arranged in the following order:
first coding region is upstream of the second coding region; and/or
(b) the order of the first coding region and the second coding region is different as compared to a corresponding order present in a reference polynucleotide, wherein the reference polynucleotide comprises a naturally-existing corresponding polynucleotide.

4. (canceled)

5. The polynucleotide of claim 1, wherein the single ORF further comprises one or more additional nucleotide sequences encoding an additional antigen (“additional coding region”), wherein the additional antigen is: (i) not the same as the first antigen, (ii) not the same as the second antigen, or (iii) not the same as both the first antigen and the second antigen.

6. (canceled)

7. The polynucleotide of claim 5, wherein the single ORF comprises at least two additional coding regions, at least three additional coding regions, at least four additional coding regions, at least five additional coding regions, at least six additional coding regions, at least seven additional coding regions, at least eight additional coding regions, at least nine additional coding regions, or at least ten additional coding regions, and
wherein the additional coding regions (a) are linked to the first coding region or to the second coding region; and/or (b) the additional coding region is linked to the first coding region or to the second coding region by a linker.

8-9. (canceled)

10. The polynucleotide of claim 5, wherein the first coding region, the second coding region, and the additional coding region are arranged in the following order:
(a) (first coding region)-L1-(second coding region)-L2-(additional coding region);
(b) (first coding region)-L1-(additional coding region)-L2-(second coding region); or
(c) (additional coding region)-L1-(first coding region)-L2-(second coding region);
wherein L1 is a first linker and L2 is a second linker, and
wherein the first linker and the second linker are the same or the first linker and the second linker are not the same.

11-12. (canceled)

13. The polynucleotide of claim 10, wherein the order of the first coding region, the second coding region, and the third coding region is different as compared to a corresponding order present in the reference polynucleotide.

14-16. (canceled)

17. The polynucleotide of claim 10, wherein:
(a) the first antigen comprises a cancer antigen, a non-self antigen, a self-antigen associated with a tumor, a disease associated-antigen, or combinations thereof; and/or
(b) the second antigen comprises a cancer antigen, a non-self antigen, a self-antigen associated with a tumor, a disease associated-antigen, or combinations thereof.

18-19. (canceled)

20. The polynucleotide of claim 17, wherein:
(a) the cancer antigen comprises a KRAS antigen; and/or
(b) the non-self antigen is derived from a pathogen selected from a human papillomavirus (HPV) antigen, a human immunodeficiency virus (HIV) antigen, a hepatitis B virus (HBV) antigen, or combinations thereof.

21-25. (canceled)

26. The polynucleotide of claim 2, wherein the linker comprises a peptide linker, optionally wherein the peptide linker comprises a G₄S linker or an EAAAK linker.

27-71. (canceled)

72. The polynucleotide of claim 1, which further comprises one or more of the following components: (1) an Internal Ribosome Entry Site (IRES), (2) an intron sequence, (3) a homology arm, (4) a promoter, (5) an enhancer, (6) a UTR, (7) a sequence encoding a signal peptide, (8) a translation initiation sequence, (9) a 3′ tailing region of linked nucleosides, (10) a 5′ cap, (11) a sequence encoding a 2A ribosome skip peptide, or (12) any combination of (1) to (11).

73. The polynucleotide of claim 1, which comprises at least one modified nucleoside, wherein the at least one modified nucleoside comprises 6-aza-cytidine, 2-thio-cytidine, α-thio-cytidine, pseudo-iso-cytidine, 5-aminoallyl-uridine, 5-iodo-uridine, N1-methyl-pseudouridine, 5,6-dihydrouridine, α-thio-uridine, 4-thio-uridine, 6-aza-uridine, 5-hydroxy-uridine, deoxy-thymidine, pseudo-uridine, inosine, α-thio-guanosine, 8-oxo-guanosine, O6-methyl-guanosine, 7-deaza-guanosine, N1-methyl adenosine, 2-amino-6-chloro-purine, N6-methyl-2-amino-purine, 6-chloro-purine, N6-methyl-adenosine, α-thio-adenosine, 8-azido-adenosine, 7-deaza-adenosine, pyrrolo-cytidine, 5-methyl-cytidine, N4-acetyl-cytidine, 5-methyl-uridine, 5-iodo-cytidine, or combinations thereof.

74-76. (canceled)

77. A cell comprising the polynucleotide of claim 1.

78. The cell of claim 77, wherein the cell comprises a stem cell, somatic cell, or both.

79. The cell of claim 78, wherein
(a) the stem cell comprises an induced pluripotent stem cell (iPSC), embryonic stem cell, tissue-specific stem cell, mesenchymal stem cell, or combinations thereof; and/or
(b) the somatic cell comprises a blood cell; and/or
(c) the somatic cell comprises a PBMC, and wherein the PBMC comprises a T cell, B cell, natural killer (NK) cell, dendritic cell (DC), NKT cell, mast cell, monocyte, macrophage, basophil, eosinophil, a neutrophil, DC2.4 dendritic cell, or combinations thereof.

80-83. (canceled)

84. The cell of claim 77, which has been passed through a constriction under a set of parameters, thereby causing a perturbation within the cell such that the polynucleotide entered the cell through the perturbation when contacted with the cell.

85-87. (canceled)

88. A method of inducing the expression of multiple antigens in a cell comprising intracellularly delivering the polynucleotide of claim 1 to the cell, wherein the multiple antigens are concurrently expressed in the cell after the intracellularly delivering.

89. (canceled)

90. The method of claim 88, wherein intracellularly delivering the polynucleotide to the cell comprises passing a cell suspension comprising the cell through a constriction under a set of parameters, thereby causing a perturbation within the cell such that the polynucleotide enters the cell through the perturbation when contacted with the cell.

91. The method of claim 90, further comprising contacting the cell with the polynucleotide, wherein
(a) the contacting occurs prior to passing the cell suspension through the constriction; and/or
(b) the contacting occurs during the passing of the cell suspension through the constriction;
and/or
(c) the contacting occurs after the cell suspension passes through the constriction.

92-99. (canceled)

100. The method of claim 90, wherein:
(a) a diameter of the constriction is about 20%, about 30%, about 40%, about 50%, about 60%, about 70%, about 80%, about 90%, or about 99% of the diameter of the cell;
and/or
(b) a width of the constriction is:
(i) between about 0 μm to about 10 μm; or
(ii) less than about 1 μm, less than about 2 μm, less than about 3 μm, less than about 4 μm, less than about 5 μm, less than about 6 μm, less than about 7 μm, less than about 8 μm, less than about 9 μm, or less than about 10 μm.

101-121. (canceled)
